(19) United States
(12) Patent Application Publication
(10) Pub. No.: US 2023/0242494 A1
(43) Pub. Date: Aug. 3, 2023

(54) MALONITRILE DERIVATIVES

(71) Applicant: Hoffmann-La Roche Inc., Little Falls, NJ (US)

(72) Inventors: Guillaume DÉCORET, Eschentzwiller (FR); Guido GALLEY, Rheinfelden (DE); Katrin GROEBKE ZBINDEN, Liestal (CH); Nicole GROSSMANN, Sisseln (CH); Wolfgang GUBA, Müllheim (DE); Daniel HUNZIKER, Möhlin (CH)

(73) Assignee: Hoffmann-La Roche Inc., Little Falls, NJ (US)

(21) Appl. No.: 17/999,195

(22) PCT Filed: May 18, 2021
(86) PCT No.: PCT/EP2021/063041

§ 371 Date: Nov. 17, 2022

(30) Foreign Application Priority Data
May 20, 2020 (EP) 20175648.3

(51) Int. Cl. C07D 261/08; C07D 413/12; C07D 417/12

(52) U.S. Cl. CPC C07D 261/08; C07D 413/12; C07D 417/12

(57) ABSTRACT

The invention relates to a compound of formula (I) wherein R¹-R⁴ and A¹-A² are as defined in the description and in the claims. The compound of formula (I) can be used as a medicament.

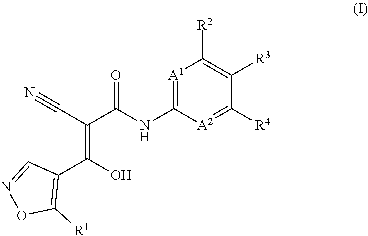

The present invention relates to organic compounds useful for therapy and/or prophylaxis in a mammal, and in particular to compounds that modulate cGAS activity.

The invention relates in particular to a compound of formula (I)

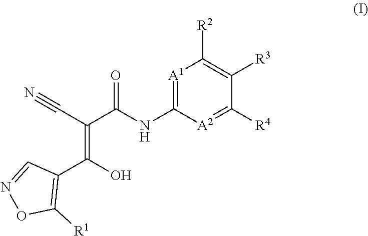

wherein
A¹ and A² are independently selected from nitrogen and —CR⁵—;
R¹ is hydrogen or alkyl;
R² and R⁴ are independently selected from hydrogen, halogen, alkyl, haloalkyl, haloalkyloxy, hydroxyalkyl, alkoxy, phenyl, halophenyl, phenylalkyl, phenylsulfonyl, phenyloxy, cyano, carboxyl, alkoxycarbonyl, alkylsulfonyl, halophenylsulfonylamino, phenylaminocarbonyl and phenylcarbonylamino;
R³ is hydrogen, halogen, haloalkyl, alkoxy, haloalkyloxy, alkoxyalkoxy, cyano, phenyl, phenylalkyl, phenyloxy, alkoxyphenyl, alkylsulfonyl, phenylsulfonyl, phenyl(alkylamino)carbonyl, phenylalkyl(alkylamino)carbonyl, alkoxypyridinylalkyl, phenylsulfanyl, phenylalkylaminocarbonyl, phenylaminocarbonyl, alkoxyalkylsulfonyl, cycloalkyl, cycloalkyloxy, alkoxyalkylaminosulfonyl, alkoxyalkyl(alkylamino)sulfonyl, dialkylaminoalkyl(alkylamino)sulfonyl, alkylaminosulfonyl, phenylsulfinyl, halophenylalkylsulfonyl, cycloalkylalkyl, phenylalkynyl, cycloalkylalkoxy, cycloalkylalkyl(alkylamino)carbonyl, phenylalkylpyrrolidinylaminocarbonyl, halophenyloxy, alkylsulfinyl, (alkylisoxazolylcarbonyl)(dialkylisoxazolyl)aminosulfonyl, dialkylisoxazolylaminosulfonyl, (alkylisoxazolylcarbonyl)(cyanophenyl)aminosulfonyl, cyanophenylaminosulfonyl, phenylsulfonimidoyl, phenylaminosulfonyl, dialkylaminocarbonylalkylaminosulfonyl, morpholinylsulfonyl, pyridinylsulfonyl, cyanophenylsulfonyl, pyrimidinylaminosulfonyl, aminocarbonyl, alkylthiadiazolylaminosulfonyl, alkylcarbonylamino, oxetanyloxy, thiazolylaminosulfonyl, alkylsulfonimidoyl, alkoxyphenylsulfonyl, phenylcarbonyl, alkylaminocarbonyl, dialkylaminocarbonyl, carboxylalkyl, alkoxycarbonyl, alkoxycarbonylalkyl, pyrrolidinylsulfonyl, dialkylaminosulfonyl, 1H-pyrrolyl, haloalkylphenyloxy, halophenylsulfonyl, alkylsulfanyl, halophenylsulfanyl or alkylcarbonyl; and
R⁵ is hydrogen, halogen, alkyl or haloalkyl;
or a pharmaceutically acceptable salt, stereoisomer or tautomer thereof.

Cytokines are responsible for modulation of the innate immune response and the dysregulation of pro-inflammatory cytokines has been associated with severe systemic inflammation and autoimmune diseases, many of which lack efficient therapy as of today.

Vertebrates possess an innate and adaptive immune system as protection against pathogens and other challenges. The innate immune system is an evolutionary old system that is present beyond vertebrates. Unlike the adaptive immune system, it does not require priming or training, but works as a general physical barrier (e.g. skin) or by detection of specific patterns. One universal pattern to trigger the innate immune system is the detection of cytosolic double stranded DNA, which leads to Type I Interferon response. Sources of cytosolic dsDNA could be from bacterial or viral infection but as well accumulated self-DNA.

The cytosolic enzyme cyclic GMP-AMP Synthase (cGAS) is a sensor for cytosolic double stranded DNA. Binding of dsDNA results in the generation of the cyclic di-nucleotide 2,3-cGAMP by enzymatic linkage of ATP and GTP. 2,3-cGAMP acts as secondary messenger and binds to the Stimulator of Interferon Genes (STING), which resides in the endoplasmatic reticulum. Upon binding of 2,3-cGAMP, STING translocates to the perinuclear Golgi, where it associates with the TANK binding kinase 1 (TBK1) and recruits and phosphorylates Interferon Response Factor 3 (IRF3). Ultimately this results in the production Type I Interferon (I IFN), other cytokines like IL-6, TNFα, IL1β and chemokines—essential factors for host defense against invading pathogens. However, inappropriate or chronic production of type I IFN and other pro-inflammatory cytokines are associated with severe systemic inflammation and autoimmune diseases. For instance, IFN signaling is involved in SLE, cutaneous skin diseases (dermatomyositis, and cutaneous lupus), interstitial pulmonary fibrosis, Sjogren syndrome, and type I diabetes (G. Trinchieri, J Exp Med. 2010 207(10): 2053-63). Other pro-inflammatory cytokine such as TNFα and IL1β play an important role in inflammatory bowel disease, NASH, juvenile inflammatory arthritis, ankylosing spondylitis and gout.

Chronic activation of cGAS/STING causes severe systemic inflammation. Evidence for its role in inflammation in the clinic comes from monogenic diseases. Patients with deficiencies in nucleic acid modifying enzymes, like Trex1, RNaseH2 and SAMHD1, suffer from Aicardi-Goutieres syndrome (AGS). The involvement of cGAS/STING was supported in Trex1 deficient mice that serve as a model for AGS.

Inhibition of the cGAS pathway which is upstream from the disease mediating cytokines is therefore a novel strategy in treating patients from multiple autoimmune diseases. Indications could include those linked to IFN signaling or those driven by TNFα and IL1β.

As of today many diseases caused by dysregulation of the innate immune system lack efficient therapies.

The compound of the invention binds to cGAS and modulates its activity.

The compound of formula (I) is particularly useful in the treatment or prophylaxis of e.g. systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C1-C8 alkyl groups are methyl, ethyl, propyl, isopropyl, butyl, isobutyl, tert.-butyl, sec.-butyl, the isomeric pentyls, the isomeric hexyls, the isomeric heptyls and the isomeric octyls, particularly methyl, ethyl, propyl, butyl and pentyl. Particular examples of alkyl are methyl, ethyl, propyl, isopropyl, and tert.-butyl. Methyl and ethyl are particular examples of “alkyl” in the compound of formula (I).

The term “cycloalkyl”, alone or in combination, signifies a cycloalkyl ring with 3 to 8 carbon atoms and particularly a cycloalkyl ring with 3 to 6 carbon atoms. Examples of cycloalkyl are cyclopropyl, cyclobutyl, cyclopentyl and cyclohexyl, cycloheptyl and cyclooctyl. Particular examples of “cycloalkyl” are cyclopropyl, cyclopentyl and cyclohexyl.

The term “alkoxy” or “alkyloxy”, alone or in combination, signifies a group of the formula alkyl-O— in which the term “alkyl” has the previously given significance, such as methoxy, ethoxy, n-propoxy, isopropoxy, n-butoxy, isobutoxy, sec-butoxy and tert.-butoxy. Particular examples of “alkoxy” are methoxy and ethoxy.

The term “oxy”, alone or in combination, signifies the —O— group.

The terms “halogen” or “halo”, alone or in combination, signifies fluorine, chlorine, bromine or iodine and particularly fluorine, chlorine or bromine, more particularly fluorine. The term “halo”, in combination with another group, denotes the substitution of said group with at least one halogen, particularly substituted with one to five halogens, particularly one to four halogens, i.e. one, two, three or four halogens.

The term “haloalkyl”, alone or in combination, denotes an alkyl group substituted with at least one halogen, particularly substituted with one to five halogens, particularly one to three halogens. Particular “haloalkyl” are trifluoromethyl, trifluoroethyl, and fluoroethyl.

The term “haloalkoxy”, alone or in combination, denotes an alkoxy group substituted with at least one halogen, particularly substituted with one to five halogens, particularly one to three halogens. Particular “haloalkoxy” are trifluoromethoxy, trifluoroethoxy, and fluoroethoxy.

The terms “hydroxyl” and “hydroxy”, alone or in combination, signify the —OH group.

The term “carbonyl”, alone or in combination, signifies the —C(O)— group.

The term “amino”, alone or in combination, signifies the primary amino group (—NH₂), the secondary amino group (—NH—), or the tertiary amino group (—N—).

The term “sulfonyl”, alone or in combination, signifies the —SO₂— group.

The term “sulfinyl”, alone or in combination, signifies the —SO— group.

The term “sulfanyl”, alone or in combination, signifies the —S— group.

The term “cyano”, alone or in combination, signifies the —CN group.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of compounds of formula (I) are the salts of hydrochloric acid, hydrobromic acid, sulfuric acid, phosphoric acid and methanesulfonic acid.

The compound of formula (I) can exist as a tautomer (I′), i.e. a structural isomer which interconverts with the the compound of formula (I), in particular in solution.

The tautomeric equilibrium of the compound of formula (I) with its tautomeric form (I′) can be represented as follows:

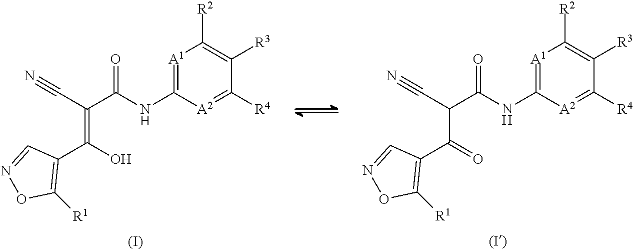

The compound of formula (I) can exist as a stereoisomer (I″), i.e. a structural isomer which interconverts with the the compound of formula (I), in particular in solution.

The isomeric equilibrium of the compound of formula (I) with its stereoisomeric form (I″) can be represented as follows:

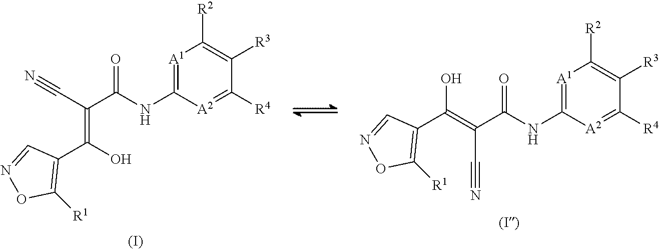

If one of the starting materials or compounds of formula (I) contain one or more functional groups which are not stable or are reactive under the reaction conditions of one or more reaction steps, appropriate protecting groups (as described e.g. in “Protective Groups in Organic Chemistry” by T. W. Greene and P. G. M. Wuts, 3^{rd} Ed., 1999, Wiley, New York) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

The compound of formula (I) can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

The term “asymmetric carbon atom” means a carbon atom with four different substituents. According to the Cahn-Ingold-Prelog Convention an asymmetric carbon atom can be of the “R” or “S” configuration.

The invention thus relates to:

A compound according to the invention wherein A¹ and A² are both nitrogen or —CR⁵— at the same time;

A compound according to the invention wherein R¹ is alkyl;

A compound according to the invention wherein R¹ is methyl;

A compound according to the invention wherein R² and R⁴ are independently selected from hydrogen and halophenyl;

A compound according to the invention wherein R² and R⁴ are independently selected from hydrogen, chlorophenyl and fluorophenyl;

A compound according to the invention wherein R³ is phenylsulfonyl, alkylsulfonyl, halogen, phenylsulfinyl, phenylalkylaminocarbonyl, alkoxyalkylsulfonyl, alkoxyalkylaminosulfonyl, alkoxyalkyl(alkylamino)sulfonyl, phenylalkyl(alkylamino)carbonyl, phenyl(alkylamino)carbonyl, alkoxy, dialkylaminosulfonyl, haloalkyl, alkoxyalkoxy or pyrimidinylaminosulfonyl;

A compound according to the invention wherein R³ is phenylsulfonyl, methylsulfonyl, ethylsulfonyl, bromo, phenylsulfinyl, phenylmethylaminocarbonyl, methoxyethylsulfonyl, methoxyethylaminosulfonyl, methoxyethyl(methylamino)sulfonyl, phenylmethyl(methylamino)carbonyl, phenyl(methylamino)carbonyl, methoxy, ethoxy, dimethylaminosulfonyl, trifluoromethyl, methoxyethoxy or pyrimidinylaminosulfonyl.

The invention further relates to a compound selected from
(Z)—N-(4-(2-chlorophenyl)-5-(methylsulfonyl)pyrimidin-2-yl)-2-cyano-3-hydroxy-3-(5-methylisoxazol-4-yl)acrylamide;
(Z)-2-cyano-N-(4-(3-fluorophenyl)-5-(phenylsulfonyl)pyrimidin-2-yl)-3-hydroxy-3-(5-methylisoxazol-4-yl)acrylamide;
(Z)—N-(4-(3-chlorophenyl)-5-(methylsulfonyl)pyrimidin-2-yl)-2-cyano-3-hydroxy-3-(5-methylisoxazol-4-yl)acrylamide;
(Z)—N-(4-chloro-6-((4-chlorophenyl)sulfonamido)-5-phenylpyrimidin-2-yl)-2-cyano-3-hydroxy-3-(5-methylisoxazol-4-yl)acrylamide;
(Z)-2-(2-cyano-3-hydroxy-3-(5-methylisoxazol-4-yl)acrylamido)-N-methyl-N,4-diphenylpyrimidine-5-carboxamide;
(Z)-2-(2-cyano-3-hydroxy-3-(5-methylisoxazol-4-yl)acrylamido)-N-methyl-4-phenethyl-N-phenylpyrimidine-5-carboxamide;
(Z)—N-benzyl-2-(2-cyano-3-hydroxy-3-(5-methylisoxazol-4-yl)acrylamido)-N-methyl-4-phenethylpyrimidine-5-carboxamide;
(Z)—N-benzyl-2-(2-cyano-3-hydroxy-3-(5-methylisoxazol-4-yl)acrylamido)-N-methyl-4-phenylpyrimidine-5-carboxamide;
(Z)—N-(5-bromopyrimidin-2-yl)-2-cyano-3-hydroxy-3-(5-methylisoxazol-4-yl)acrylamide;
(Z)-2-cyano-3-hydroxy-3-(5-methylisoxazol-4-yl)-N-(5-(methylsulfonyl)-4-phenethylpyrimidin-2-yl)acrylamide;
(Z)-2-cyano-3-hydroxy-N-(5-methoxy-4-phenylpyrimidin-2-yl)-3-(5-methylisoxazol-4-yl)acrylamide;
(Z)-2-cyano-3-hydroxy-3-(5-methylisoxazol-4-yl)-N-(5-phenethyl-4-(trifluoromethyl)pyrimidin-2-yl)acrylamide;
(Z)-2-cyano-3-hydroxy-N-(5-(2-(6-methoxypyridin-3-yl)ethyl)-4-(trifluoromethyl)pyrimidin-2-yl)-3-(5-methylisoxazol-4-yl)acrylamide;
(Z)-2-cyano-3-hydroxy-N-(5-methoxy-4-phenethylpyrimidin-2-yl)-3-(5-methylisoxazol-4-yl)acrylamide;
(Z)-2-cyano-3-hydroxy-N-(5-(2-methoxyethoxy)-4-phenethylpyrimidin-2-yl)-3-(5-methylisoxazol-4-yl)acrylamide;
(Z)-2-cyano-3-hydroxy-N-(5-(2-methoxyethoxy)-4-phenylpyrimidin-2-yl)-3-(5-methylisoxazol-4-yl)acrylamide;
(Z)—N-[5-(benzenesulfinyl)pyrimidin-2-yl]-2-cyano-3-hydroxy-3-(5-methylisoxazol-4-yl)prop-2-enamide;
(Z)—N-[5-(benzenesulfonyl)pyrimidin-2-yl]-2-cyano-3-hydroxy-3-(5-methylisoxazol-4-yl)prop-2-enamide;
N-benzyl-2-[[(Z)-2-cyano-3-hydroxy-3-(5-methylisoxazol-4-yl)prop-2-enoyl]amino]pyrimidine-5-carboxamide;
2-[[(Z)-2-cyano-3-hydroxy-3-(5-methylisoxazol-4-yl)prop-2-enoyl]amino]-N-phenyl-pyrimidine-5-carboxamide;
(Z)-2-cyano-3-hydroxy-3-(5-methylisoxazol-4-yl)-N-(4-(2-phenylpropan-2-yl)phenyl)acrylamide;
(Z)-2-cyano-N-(5-(ethylsulfonyl)pyrimidin-2-yl)-3-hydroxy-3-(5-methylisoxazol-4-yl)acrylamide;
(Z)-2-cyano-3-hydroxy-3-(5-methylisoxazol-4-yl)-N-(5-phenethylpyrimidin-2-yl)acrylamide;
(Z)-2-cyano-3-hydroxy-N-(5-((2-methoxyethyl)sulfonyl)pyrimidin-2-yl)-3-(5-methylisoxazol-4-yl)acrylamide;
(Z)—N-(4-(4-chlorophenyl)-5-(cyclopropylmethoxy)pyrimidin-2-yl)-2-cyano-3-hydroxy-3-(5-methylisoxazol-4-yl)acrylamide;
(Z)—N-(4-(4-chlorophenyl)-5-(2,2,2-trifluoroethoxy)pyrimidin-2-yl)-2-cyano-3-hydroxy-3-(5-methylisoxazol-4-yl)acrylamide;
(Z)-2-cyano-3-hydroxy-N-(5-(N-(2-methoxyethyl)sulfamoyl)pyrimidin-2-yl)-3-(5-methylisoxazol-4-yl)acrylamide;
(Z)-2-cyano-3-hydroxy-N-(5-(N-(2-methoxyethyl)-N-methylsulfamoyl)pyrimidin-2-yl)-3-(5-methylisoxazol-4-yl)acrylamide;
(Z)-2-cyano-N-(4-(N-(2-(dimethylamino)ethyl)-N-methylsulfamoyl)phenyl)-3-hydroxy-3-(5-methylisoxazol-4-yl)acrylamide;
(Z)-2-cyano-N-(4-(3-fluorophenyl)-5-(methylsulfonyl)pyrimidin-2-yl)-3-hydroxy-3-(5-methylisoxazol-4-yl)acrylamide;
(Z)-2-cyano-N-(4-(2-fluoroethoxy)pyrimidin-2-yl)-3-hydroxy-3-(5-methylisoxazol-4-yl)acrylamide;
N-benzyl-2-[[(Z)-2-cyano-3-hydroxy-3-(5-methylisoxazol-4-yl)prop-2-enoyl]amino]-N-methyl-pyrimidine-5-carboxamide;
2-[[(Z)-2-cyano-3-hydroxy-3-(5-methylisoxazol-4-yl)prop-2-enoyl]amino]-N-methyl-N-phenyl-pyrimidine-5-carboxamide;
(Z)-2-cyano-N-(5-ethoxypyrimidin-2-yl)-3-hydroxy-3-(5-methylisoxazol-4-yl)acrylamide;
(Z)-2-cyano-3-hydroxy-3-(5-methylisoxazol-4-yl)-N-(5-phenylsulfanylpyrimidin-2-yl)prop-2-enamide;
(Z)-2-cyano-N-(5-(N,N-dimethylsulfamoyl)pyrimidin-2-yl)-3-hydroxy-3-(5-methylisoxazol-4-yl)acrylamide;
(Z)-2-cyano-3-hydroxy-3-(5-methylisoxazol-4-yl)-N-(5-(phenylsulfonyl)pyridin-2-yl)acrylamide;
(Z)-2-cyano-3-hydroxy-3-(5-methylisoxazol-4-yl)-N-(5-(trifluoromethyl)pyrimidin-2-yl)acrylamide;
(Z)—N-(5-(N-(tert-butyl)sulfamoyl)pyridin-2-yl)-2-cyano-3-hydroxy-3-(5-methylisoxazol-4-yl)acrylamide;
(Z)-2-cyano-N-[3-fluoro-5-(trifluoromethyl)-2-pyridyl]-3-hydroxy-3-(5-methylisoxazol-4-yl)prop-2-enamide;
(Z)-2-cyano-3-hydroxy-3-(5-methylisoxazol-4-yl)-N-(5-(methylsulfonyl)pyridin-2-yl)acrylamide;
(Z)-2-cyano-3-hydroxy-3-(5-methylisoxazol-4-yl)-N-(5-(phenylsulfinyl)pyridin-2-yl)acrylamide;
(Z)-2-cyano-3-hydroxy-N-(5-(2-methoxyethoxy)pyrimidin-2-yl)-3-(5-methylisoxazol-4-yl)acrylamide;
(Z)-2-cyano-3-hydroxy-N-(4-(N-(2-methoxyethyl)-N-methylsulfamoyl)phenyl)-3-(5-methylisoxazol-4-yl)acrylamide;
(Z)—N-(5-((2-chlorobenzyl)sulfonyl)pyridin-2-yl)-2-cyano-3-hydroxy-3-(5-methylisoxazol-4-yl)acrylamide;
(Z)-2-cyano-N-(5-(cyclohexylmethyl)pyrimidin-2-yl)-3-hydroxy-3-(5-methylisoxazol-4-yl)acrylamide;
(Z)-2-cyano-3-hydroxy-3-(5-methylisoxazol-4-yl)-N-(4-phenethylpyrimidin-2-yl)acrylamide;
(Z)-2-cyano-3-hydroxy-3-(5-methylisoxazol-4-yl)-N-(5-(phenylethynyl)pyrimidin-2-yl)acrylamide;
(Z)-2-cyano-N-(5-(cyclopentylmethoxy)pyrimidin-2-yl)-3-hydroxy-3-(5-methylisoxazol-4-yl)acrylamide;
(Z)—N-(3-chloro-5-(trifluoromethyl)pyridin-2-yl)-2-cyano-3-hydroxy-3-(5-methylisoxazol-4-yl)acrylamide;
(Z)-2-cyano-3-hydroxy-3-(5-methylisoxazol-4-yl)-N-(5-phenoxypyridin-2-yl)acrylamide;
(Z)-2-cyano-3-hydroxy-3-(5-methylisoxazol-4-yl)-N-(5-phenoxypyrimidin-2-yl)acrylamide;
(Z)-2-cyano-3-hydroxy-3-(5-methylisoxazol-4-yl)-N-(5-phenylpyrimidin-2-yl)acrylamide;
(Z)-2-cyano-3-hydroxy-N-(5-(4-methoxyphenyl)pyrimidin-2-yl)-3-(5-methylisoxazol-4-yl)acrylamide;
(Z)-2-cyano-N-(5-cyclohexylpyrimidin-2-yl)-3-hydroxy-3-(5-methylisoxazol-4-yl)acrylamide;
(Z)-2-cyano-N-(3-cyano-4-(trifluoromethyl)phenyl)-3-hydroxy-3-(5-methylisoxazol-4-yl)acrylamide;
(Z)-2-chloro-4-(2-cyano-3-hydroxy-3-(5-methylisoxazol-4-yl)acrylamido)-N-(cyclohexylmethyl)-N-methylbenzamide;
(Z)-2-cyano-3-hydroxy-N-(3-methoxy-4-(trifluoromethyl)phenyl)-3-(5-methylisoxazol-4-yl)acrylamide;
(Z)-2-cyano-3-hydroxy-N-(3-methyl-4-(trifluoromethyl)phenyl)-3-(5-methylisoxazol-4-yl)acrylamide;
(Z)—N-(4-benzylphenyl)-2-cyano-3-hydroxy-3-(5-methylisoxazol-4-yl)acrylamide;
(Z)-2-cyano-N-(5-(cyclohexyloxy)pyrimidin-2-yl)-3-hydroxy-3-(5-methylisoxazol-4-yl)acrylamide;
(Z)—N-benzyl-4-(2-cyano-3-hydroxy-3-(5-methylisoxazol-4-yl)acrylamido)benzamide;
(Z)—N-(3-(2-cyano-3-hydroxy-3-(5-methylisoxazol-4-yl)acrylamido)phenyl)benzamide;
(S,Z)—N-(1-benzylpyrrolidin-3-yl)-4-(2-cyano-3-hydroxy-3-(5-methylisoxazol-4-yl)acrylamido)benzamide hydrochloride;
(Z)-3-(2-cyano-3-hydroxy-3-(5-methylisoxazol-4-yl)acrylamido)-N-phenylbenzamide;
(Z)-2-cyano-3-hydroxy-3-(5-methylisoxazol-4-yl)-N-(4-phenylpyrimidin-2-yl)acrylamide;
(Z)-4-(2-cyano-3-hydroxy-3-(5-methylisoxazol-4-yl)acrylamido)-N-phenylbenzamide;
(Z)-2-cyano-3-hydroxy-3-(5-methylisoxazol-4-yl)-N-(5-(trifluoromethyl)pyridin-2-yl)acrylamide;
(Z)—N-(3-chloro-4-(trifluoromethyl)phenyl)-2-cyano-3-hydroxy-3-(5-methylisoxazol-4-yl)acrylamide;
(Z)-2-cyano-3-hydroxy-3-(5-methylisoxazol-4-yl)-N-(4-phenylphenyl)prop-2-enamide;
(Z)—N-(4-(4-chlorophenoxy)phenyl)-2-cyano-3-hydroxy-3-(5-methylisoxazol-4-yl)acrylamide;
(Z)-2-cyano-3-hydroxy-3-(5-methylisoxazol-4-yl)-N-(4-phenoxyphenyl)acrylamide;
(Z)-2-cyano-3-hydroxy-3-(5-methylisoxazol-4-yl)-N-(4-methylsulfinylphenyl)prop-2-enamide;
(Z)-2-cyano-3-hydroxy-3-(5-methylisoxazol-4-yl)-N-(4-methylsulfonylphenyl)prop-2-enamide;
(Z)—N-(2-chloro-4-methylsulfonyl-phenyl)-2-cyano-3-hydroxy-3-(5-methylisoxazol-4-yl)prop-2-enamide;
(Z)—N-((4-(2-cyano-3-hydroxy-3-(5-methylisoxazol-4-yl)acrylamido)phenyl)sulfonyl)-N-(3,5-dimethylisoxazol-4-yl)-5-methylisoxazole-4-carboxamide;
(Z)-2-cyano-N-(4-(N-(3,5-dimethylisoxazol-4-yl)sulfamoyl)phenyl)-3-hydroxy-3-(5-methylisoxazol-4-yl)acrylamide;
(Z)—N-((4-(2-cyano-3-hydroxy-3-(5-methylisoxazol-4-yl)acrylamido)phenyl)sulfonyl)-N-(4-cyanophenyl)-5-methylisoxazole-4-carboxamide;
(Z)-2-cyano-N-(4-(N-(4-cyanophenyl)sulfamoyl)phenyl)-3-hydroxy-3-(5-methylisoxazol-4-yl)acrylamide;
(Z)-2-cyano-3-hydroxy-3-(5-methylisoxazol-4-yl)-N-(4-(phenylsulfonimidoyl)phenyl)acrylamide;
(Z)—N-(2-chloro-4-(trifluoromethyl)phenyl)-2-cyano-3-hydroxy-3-(5-methylisoxazol-4-yl)acrylamide;
(Z)-2-cyano-3-hydroxy-3-(5-methylisoxazol-4-yl)-N-(4-(phenylthio)phenyl)acrylamide;
(Z)-2-cyano-3-hydroxy-3-(5-methylisoxazol-4-yl)-N-(4-(N-phenylsulfamoyl)phenyl)acrylamide;
(Z)-2-cyano-N-(4-(N-(2-(diethylamino)-2-oxoethyl)sulfamoyl)phenyl)-3-hydroxy-3-(5-methylisoxazol-4-yl)acrylamide;
(Z)-2-cyano-3-hydroxy-3-(5-methylisoxazol-4-yl)-N-(3-phenoxyphenyl)acrylamide;
(Z)-2-cyano-3-hydroxy-3-(5-methylisoxazol-4-yl)-N-(3-phenylphenyl)prop-2-enamide;
(Z)-2-cyano-3-hydroxy-N-(2-methyl-4-(trifluoromethyl)phenyl)-3-(5-methylisoxazol-4-yl)acrylamide;
(Z)-2-cyano-3-hydroxy-3-(5-methylisoxazol-4-yl)-N-(3-(phenylsulfonyl)phenyl)acrylamide;
(Z)-2-cyano-3-hydroxy-3-(5-methylisoxazol-4-yl)-N-(4-(N-propylsulfamoyl)phenyl)acrylamide;
(Z)-2-cyano-3-hydroxy-3-(5-methylisoxazol-4-yl)-N-(4-(morpholinosulfonyl)phenyl)acrylamide;
(Z)-2-cyano-3-hydroxy-N-(5-methoxypyrimidin-2-yl)-3-(5-methylisoxazol-4-yl)prop-2-enamide;
(Z)-2-cyano-3-hydroxy-3-(5-methylisoxazol-4-yl)-N-(4-(pyridin-3-ylsulfonyl)phenyl)acrylamide;
(Z)-2-cyano-N-(4-((2-cyanophenyl)sulfonyl)phenyl)-3-hydroxy-3-(5-methylisoxazol-4-yl)acrylamide;
(Z)-2-cyano-N-(4-((4-cyanophenyl)sulfonyl)phenyl)-3-hydroxy-3-(5-methylisoxazol-4-yl)acrylamide;
(Z)-2-cyano-3-(5-ethylisoxazol-4-yl)-3-hydroxy-N-(4-(trifluoromethyl)phenyl)acrylamide;
(Z)-2-cyano-3-hydroxy-3-(5-methylisoxazol-4-yl)-N-[4-(pyrimidin-2-ylsulfamoyl)phenyl]prop-2-enamide;
4-[[(Z)-2-cyano-3-hydroxy-3-(5-methylisoxazol-4-yl)prop-2-enoyl]amino]benzamide;
(Z)-2-cyano-3-hydroxy-3-(5-methylisoxazol-4-yl)-N-[4-[(5-methyl-1,3,4-thiadiazol-2-yl)sulfamoyl]phenyl]prop-2-enamide;
(Z)—N-(4-acetamidophenyl)-2-cyano-3-hydroxy-3-(5-methylisoxazol-4-yl)prop-2-enamide;
(Z)-2-cyano-3-hydroxy-3-(5-methylisoxazol-4-yl)-N-[4-(oxetan-3-yloxy)phenyl]prop-2-enamide;
3-[[(Z)-2-cyano-3-hydroxy-3-(5-methylisoxazol-4-yl)prop-2-enoyl]amino]benzoic acid;
(Z)-2-cyano-3-hydroxy-3-(5-methylisoxazol-4-yl)-N-[4-(thiazol-2-ylsulfamoyl)phenyl]prop-2-enamide;
(Z)-2-cyano-3-hydroxy-3-(5-methylisoxazol-4-yl)-N-(4-(S-methylsulfonimidoyl)phenyl)acrylamide;
(Z)-2-cyano-3-hydroxy-N-(4-((4-methoxyphenyl)sulfonyl)phenyl)-3-(5-methylisoxazol-4-yl)acrylamide;
(Z)—N-(4-benzoylphenyl)-2-cyano-3-hydroxy-3-(5-methylisoxazol-4-yl)prop-2-enamide;
(Z)-2-cyano-3-hydroxy-N-(5-methoxy-2-pyridyl)-3-(5-methylisoxazol-4-yl)prop-2-enamide;
4-[[(Z)-2-cyano-3-hydroxy-3-(5-methylisoxazol-4-yl)prop-2-enoyl]amino]-N-methylbenzamide;
2-[4-[[(Z)-2-cyano-3-hydroxy-3-(5-methylisoxazol-4-yl)prop-2-enoyl]amino]phenyl]acetic acid;
4-[[(Z)-2-cyano-3-hydroxy-3-(5-methylisoxazol-4-yl)prop-2-enoyl]amino]-N,N-dimethylbenzamide;
(Z)-2-cyano-3-hydroxy-3-(5-methylisoxazol-4-yl)-N-(4-(phenylsulfinyl)phenyl)acrylamide;
(Z)-2-cyano-N-(3-cyanophenyl)-3-hydroxy-3-(5-methylisoxazol-4-yl)prop-2-enamide;
methyl 2-[4-[[(Z)-2-cyano-3-hydroxy-3-(5-methylisoxazol-4-yl)prop-2-enoyl]amino]phenyl]acetate;
(Z)-2-cyano-3-hydroxy-N-(4-methoxy-3-methyl-phenyl)-3-(5-methylisoxazol-4-yl)prop-2-enamide;
(Z)-2-cyano-3-hydroxy-3-(5-methylisoxazol-4-yl)-N-(4-(phenylsulfonyl)phenyl)acrylamide;
(Z)-2-cyano-3-hydroxy-N-(4-(N-(2-methoxyethyl)sulfamoyl)phenyl)-3-(5-methylisoxazol-4-yl)acrylamide;
(Z)-2-cyano-N-(4-ethoxyphenyl)-3-hydroxy-3-(5-methylisoxazol-4-yl)prop-2-enamide;
(Z)-2-cyano-3-hydroxy-3-(5-methylisoxazol-4-yl)-N-(4-(pyrrolidin-1-ylsulfonyl)phenyl)acrylamide;
(Z)-2-cyano-3-hydroxy-3-(5-methylisoxazol-4-yl)-N-(3-(methylsulfonyl)phenyl)acrylamide;
(Z)-2-cyano-N-(4-(N,N-dimethylsulfamoyl)phenyl)-3-hydroxy-3-(5-methylisoxazol-4-yl)acrylamide;
(Z)-2-cyano-3-hydroxy-N-[3-(hydroxymethyl)phenyl]-3-(5-methylisoxazol-4-yl)prop-2-enamide;
methyl 3-[[(Z)-2-cyano-3-hydroxy-3-(5-methylisoxazol-4-yl)prop-2-enoyl]amino]benzoate;
(Z)-2-cyano-3-hydroxy-3-(isoxazol-4-yl)-N-(4-(trifluoromethyl)phenyl)acrylamide;
(Z)—N-(3-chloro-4-(4-chlorophenoxy)phenyl)-2-cyano-3-hydroxy-3-(5-methylisoxazol-4-yl)acrylamide;
(Z)—N-(4-bromo-3-methylphenyl)-2-cyano-3-hydroxy-3-(5-methylisoxazol-4-yl)acrylamide;
(Z)-2-cyano-N-(4-cyano-2-methylphenyl)-3-hydroxy-3-(5-methylisoxazol-4-yl)acrylamide;
(Z)—N-(4-(1H-pyrrol-1-yl)phenyl)-2-cyano-3-hydroxy-3-(5-methylisoxazol-4-yl)acrylamide;
(Z)—N-(4-chlorophenyl)-2-cyano-3-hydroxy-3-(5-methylisoxazol-4-yl)acrylamide;
(Z)-2-cyano-3-hydroxy-N-(3-methyl-4-(4-(trifluoromethyl)phenoxy)phenyl)-3-(5-methylisoxazol-4-yl)acrylamide;
(Z)-2-cyano-3-hydroxy-3-(5-methylisoxazol-4-yl)-N-(4-(3-(trifluoromethyl)phenoxy)phenyl)acrylamide;
(Z)-2-cyano-3-hydroxy-N-(4-methoxyphenyl)-3-(5-methylisoxazol-4-yl)acrylamide;
(Z)—N-(4-((4-chlorophenyl)sulfonyl)phenyl)-2-cyano-3-hydroxy-3-(5-methylisoxazol-4-yl)acrylamide;
(Z)—N-(4-chloro-3-(trifluoromethyl)phenyl)-2-cyano-3-hydroxy-3-(5-methylisoxazol-4-yl)acrylamide;
(Z)-2-cyano-3-hydroxy-3-(5-methylisoxazol-4-yl)-N-(4-(methylthio)phenyl)acrylamide;
(Z)—N-(4-((4-chlorophenyl)thio)phenyl)-2-cyano-3-hydroxy-3-(5-methylisoxazol-4-yl)acrylamide;
(Z)-2-cyano-N-(3,4-dichlorophenyl)-3-hydroxy-3-(5-methylisoxazol-4-yl)acrylamide;
(Z)-2-cyano-3-hydroxy-3-(5-methylisoxazol-4-yl)-N-(4-(trifluoromethoxy)phenyl)acrylamide;
(Z)-2-cyano-N-(4-fluorophenyl)-3-hydroxy-3-(5-methylisoxazol-4-yl)acrylamide;
(Z)-2-cyano-3-hydroxy-3-(5-methylisoxazol-4-yl)-N-(2-(trifluoromethyl)phenyl)acrylamide;
methyl (Z)-4-(2-cyano-3-hydroxy-3-(5-methylisoxazol-4-yl)acrylamido)benzoate;
(Z)-2-cyano-N-(4-cyanophenyl)-3-hydroxy-3-(5-methylisoxazol-4-yl)acrylamide;
(Z)—N-(3,5-bis(trifluoromethyl)phenyl)-2-cyano-3-hydroxy-3-(5-methylisoxazol-4-yl)acrylamide;
(Z)-2-cyano-3-hydroxy-3-(5-methylisoxazol-4-yl)-N-[3-(trifluoromethyl)phenyl]prop-2-enamide;
(Z)-2-cyano-3-hydroxy-3-(5-methylisoxazol-4-yl)-N-(4-(trifluoromethyl)phenyl)acrylamide;
(Z)-2-cyano-3-hydroxy-3-(5-methylisoxazol-4-yl)-N-phenylacrylamide; and
(Z)—N-(4-acetylphenyl)-2-cyano-3-hydroxy-3-(5-methylisoxazol-4-yl)prop-2-enamide;
   or a pharmaceutically acceptable salt, stereoisomer or tautomer thereof.

The invention further relates to a compound selected from
(Z)-2-cyano-N-(4-(3-fluorophenyl)-5-(phenylsulfonyl)pyrimidin-2-yl)-3-hydroxy-3-(5-methylisoxazol-4-yl)acrylamide;
(Z)—N-(4-(3-chlorophenyl)-5-(methylsulfonyl)pyrimidin-2-yl)-2-cyano-3-hydroxy-3-(5-methylisoxazol-4-yl)acrylamide;
(Z)—N-(5-bromopyrimidin-2-yl)-2-cyano-3-hydroxy-3-(5-methylisoxazol-4-yl)acrylamide;
(Z)—N-[5-(benzenesulfinyl)pyrimidin-2-yl]-2-cyano-3-hydroxy-3-(5-methylisoxazol-4-yl)prop-2-enamide;
(Z)—N-[5-(benzenesulfonyl)pyrimidin-2-yl]-2-cyano-3-hydroxy-3-(5-methylisoxazol-4-yl)prop-2-enamide;
N-benzyl-2-[[(Z)-2-cyano-3-hydroxy-3-(5-methylisoxazol-4-yl)prop-2-enoyl]amino]pyrimidine-5-carboxamide;
(Z)-2-cyano-N-(5-(ethylsulfonyl)pyrimidin-2-yl)-3-hydroxy-3-(5-methylisoxazol-4-yl)acrylamide;
(Z)-2-cyano-3-hydroxy-N-(5-((2-methoxyethyl)sulfonyl)pyrimidin-2-yl)-3-(5-methylisoxazol-4-yl)acrylamide;
(Z)-2-cyano-3-hydroxy-N-(5-(N-(2-methoxyethyl)sulfamoyl)pyrimidin-2-yl)-3-(5-methylisoxazol-4-yl)acrylamide;
(Z)-2-cyano-3-hydroxy-N-(5-(N-(2-methoxyethyl)-N-methylsulfamoyl)pyrimidin-2-yl)-3-(5-methylisoxazol-4-yl)acrylamide;
(Z)-2-cyano-N-(4-(3-fluorophenyl)-5-(methylsulfonyl)pyrimidin-2-yl)-3-hydroxy-3-(5-methylisoxazol-4-yl)acrylamide;
N-benzyl-2-[[(Z)-2-cyano-3-hydroxy-3-(5-methylisoxazol-4-yl)prop-2-enoyl]amino]-N-methyl-pyrimidine-5-carboxamide;
2-[[(Z)-2-cyano-3-hydroxy-3-(5-methylisoxazol-4-yl)prop-2-enoyl]amino]-N-methyl-N-phenyl-pyrimidine-5-carboxamide;
(Z)-2-cyano-N-(5-ethoxypyrimidin-2-yl)-3-hydroxy-3-(5-methylisoxazol-4-yl)acrylamide;
(Z)-2-cyano-N-(5-(N,N-dimethylsulfamoyl)pyrimidin-2-yl)-3-hydroxy-3-(5-methylisoxazol-4-yl)acrylamide;
(Z)-2-cyano-3-hydroxy-3-(5-methylisoxazol-4-yl)-N-(5-(trifluoromethyl)pyrimidin-2-yl)acrylamide;
(Z)-2-cyano-3-hydroxy-N-(5-(2-methoxyethoxy)pyrimidin-2-yl)-3-(5-methylisoxazol-4-yl)acrylamide;
(Z)-2-cyano-3-hydroxy-N-(5-methoxypyrimidin-2-yl)-3-(5-methylisoxazol-4-yl)prop-2-enamide;
(Z)-2-cyano-3-hydroxy-3-(5-methylisoxazol-4-yl)-N-[4-(pyrimidin-2-ylsulfamoyl)phenyl]prop-2-enamide; and
(Z)-2-cyano-3-hydroxy-N-(4-(N-(2-methoxyethyl)sulfamoyl)phenyl)-3-(5-methylisoxazol-4-yl)acrylamide;
   or a pharmaceutically acceptable salt, stereoisomer or tautomer thereof.

The synthesis of the compound of formula (I) can, for example, be accomplished according to the following schemes.

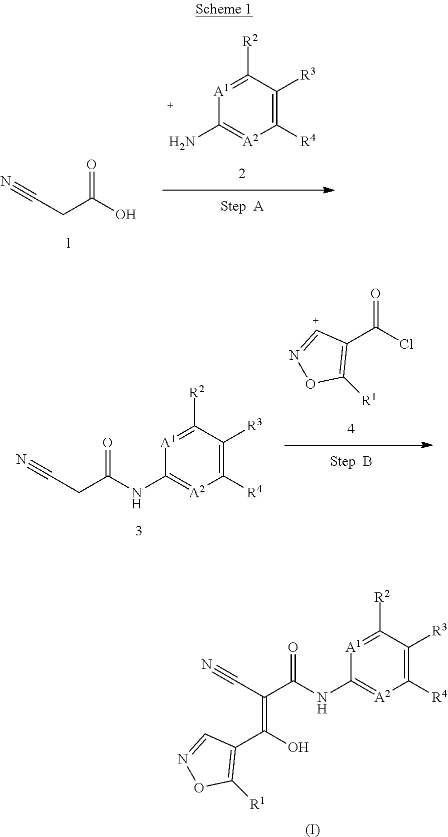

In scheme 1, R¹-R⁴, A¹ and A² are as defined above.

Step A: Cyanoacetamides 3 can be obtained by reacting a suitable amine 1 with cyano acetic acid 2, using a coupling reagent such as DCC, HATU, EDCI or propyl phosphonic anhydride optionally in the presence of a base such as triethylamine, DIPEA or pyridine in a solvent such as dichloromethane, THF, acetonitrile, ethyl acetate or DMF, or alternatively by activating the acid via the acid chloride with oxalyl chloride/DMF, thionyl chloride/DMF or methanesulfonyl chloride/3-methylpyridine optionally in the presence of a base such as triethylamine, DIPEA in a solvent such as acetonitrile, THF, dichloromethane, toluene or dioxane at between around RT—80° C. for between around 2-12 hrs.

Step B: Compounds of formula (I) can be obtained by deprotonation of cyano acetamide 3 with a base such as NaH and subsequent reaction with an acid chloride 4 in a solvent such as THF, dichloromethane or a mixture thereof. Convenient conditions are the use of NaH as a base in a mixture of THF and dichloromethane at around room temperature for between around 5-20 hrs.

Alternatively, compounds of formula (I) can be prepared by the method shown in scheme 2.

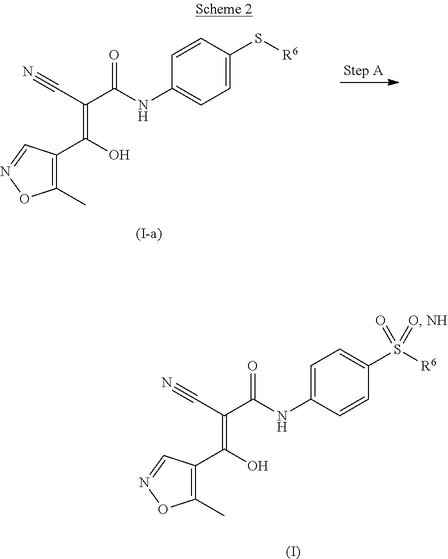

In scheme 2, R⁶ is phenyl or alkyl.

Step A: Sulfones of formula (I) can be obtained by oxidation of thioethers (I-a) (prepared by methods described in scheme 1) using an oxidant such as mCPBA in a solvent such as dichloromethane at around RT for between around 2-12 hrs.

Sulfoximines of formula (I) can be obtained from thioethers (I-a) using an oxidant such as (diacetoxyiodo)benzene in the presence of ammonium acetate in a solvent such as ethanol at around RT for between around 2-12 hrs.

The synthesis of the intermediates for preparation of the compound of formula (I) can be accomplished according to schemes 3-9.

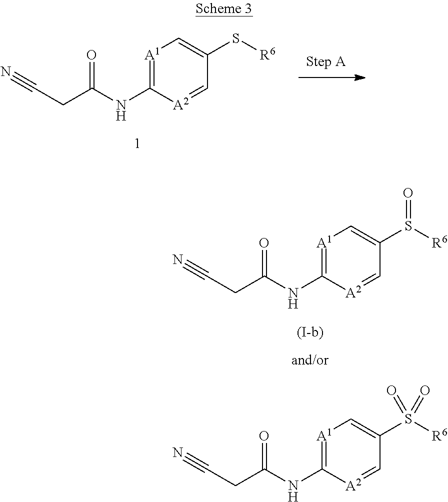

In scheme 3, A¹ and A² are as defined above; R⁶ is chlorobenzyl, phenyl or methoxyethyl.

Step A: Sulfones and sulfoxides intermediates (I-b) can be obtained from thioether intermediates 1 by reaction with an oxidant such as mCPBA or potassium hydrogenperoxomonosulphate (oxone) in a solvent such as DCM at around RT for between around 2-12 hrs. The intermediates (I-b) can be further processed to a compound of formula (I) according to step B of scheme 1.

Aminopyrimidine/aniline intermediates are either commercially available, can be prepared by the methods in shown in scheme 4 or by methods known to those skilled in the art.

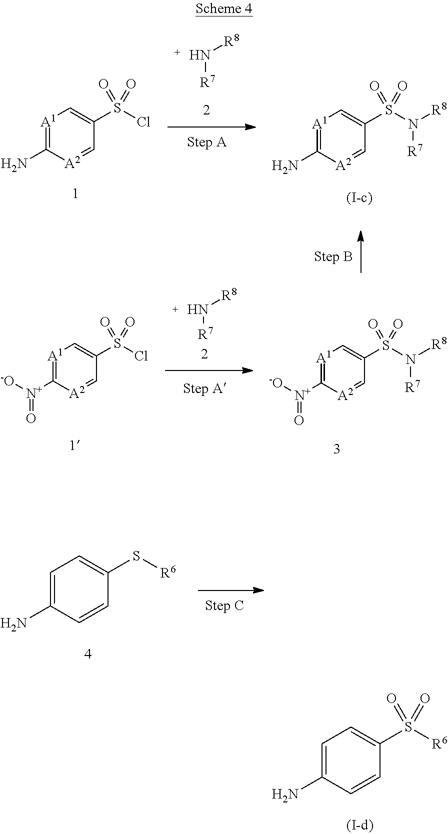

In scheme 4, A¹ and A² are as defined above; R⁶ is pyridinyl or cyanophenyl; R⁷ is methyl or hydrogen; and R⁸ is methoxyethyl or (dimethylamino)ethyl.

Step A: Sulfonamide intermediates (I-c) can be obtained by reaction of a sulfonyl chloride 1 with a suitable amine 2 in the presence of a base such as triethylamine, DIPEA in a solvent such as dichloromethane, acetonitrile, THF, DMF, NMP, pyridine at 0-50° C. for 2-16 hrs. The sulfonamide compounds (I-c) can be further processed to a compound of formula (I) according to steps A and B of scheme 1.

Step B: Intermediates (I-c) can also be obtained from nitro precursors 3 by hydrogenation using Pd/C as catalyst in a solvent such as MeOH, EtOH, THF, EtOAc at around RT for between around 2-16 hrs.

Step C: Sulfone intermediates (I-d) can be obtained from thioether intermediates 4 by oxidation of thioether by an oxidant such as mCPBA in a solvent such as DCM at around RT for between around 2-12 hrs. The intermediates (I-d) can be further processed to a compound of formula (I) according to steps A and B of scheme 1.

Aminopyrimidines are either commercially available, can be prepared by the methods shown in schemes 5 to 9 or by methods known to those skilled in the art.

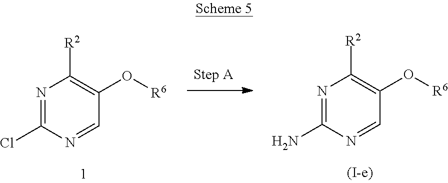

In scheme 5, R² is phenyl or chlorophenyl; R⁶ is methyl, cyclopropylmethyl, trifluoroethyl or methoxyethyl.

Step A: Aminopyrimidine intermediates (I-e) can be obtained from chloropyrimidine precursors 1 by reaction with benzophenonimine in the presence of a suitable catalyst such as Pd(II)(OAc)₂/BINAP or PdCl₂(dppf)₂ and a suitable base such as Cs₂CO₃ in a solvent such as dioxane, THF at 80-120° C. for 2-16 hrs and subsequent cleavage of the imine with hydroxylamine/sodium acetate in a solvent such as methanol at between around 0° C. —RT for between around 2-16 hrs. The intermediates (I-e) can be further processed to a compound of formula (I) according to steps A and B of scheme 1.

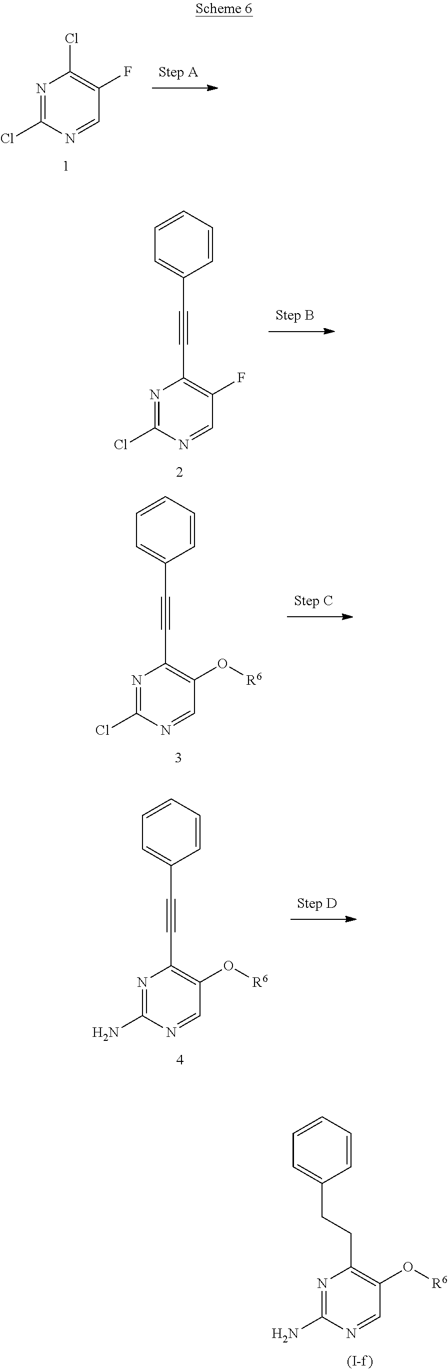

In scheme 6, R⁶ is methyl or methoxyethyl.

Step A: Acetylene intermediates 2 can be obtained by Sonogashira reaction of 2,4-dichloro-5-fluoropyrimidine 1 with a suitable acetylene with tetrakis(triphenylphosphine)-palladium(0) as catalyst, CuI as additive and triethylamine as base in a solvent such as dioxane, THE or DMF at 80-120° C.

Step B: Alkoxy intermediates 3 can be obtained by substitution on intermediate 2 with an alcoholate which can for example be generated from a suitable alcohol with NaH as base in a solvent such as DMF at between around 0° C.—RT for between around 2-16 hrs.

Step C: Aminopyrimidine intermediates 4 can be obtained from chloropyrimidine precursors 3 by reaction with benzophenonimine in the presence of a suitable catalyst such as Pd(II)(OAc)₂/BINAP and a suitable base such as Cs₂CO₃ in a solvent such as dioxane, THE at 80 120° C. for 2-16 hrs and subsequent cleavage of the imine with hydroxylamine/sodium acetate in a solvent such as methanol at 0° C.—RT for 2-16 hrs.

Step D: Aminopyrimidine intermediates (I-f) can be obtained from acetylene precursors 4 by hydrogenation using Pd/C as catalyst in a solvent such as MeOH, EtOH, THF, EtOAc at RT for 2-16 hrs. The intermediates (I-f) can be further processed to a compound of formula (I) according to steps A and B of scheme 1.

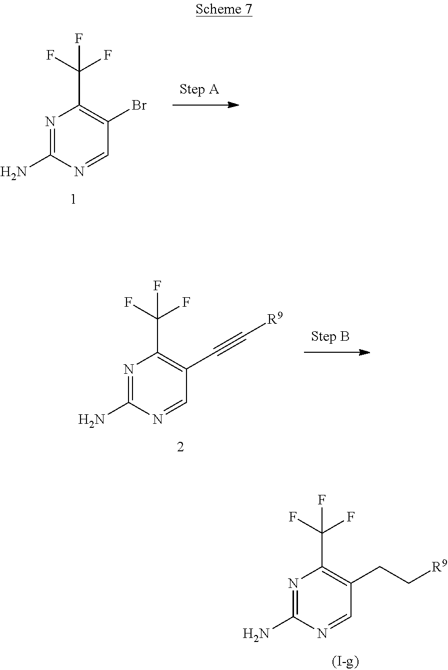

In scheme 7, R⁹ is phenyl or methoxypyridinyl.

Step A: Acetylene intermediates 2 were obtained by Sonogashira reaction of 5-bromo-4-(trifluoromethyl)pyrimidin-2-amine 1 with a suitable acetylene with tetrakis(triphenylphosphine)-palladium(0) as catalyst, CuI as additive and triethylamine as base in a solvent such as dioxane, THE or DMF at 80-120° C.

Step B: Aminopyrimidine intermediates (I-g) can be obtained from acetylene precursors 2 by hydrogenation using Pd/C as catalyst in a solvent such as MeOH, EtOH, THF, EtOAc at RT for 2-16 hrs. The intermediate compounds (I-g) can be further processed to a compound of formula (I) according to steps A and B of scheme 1.

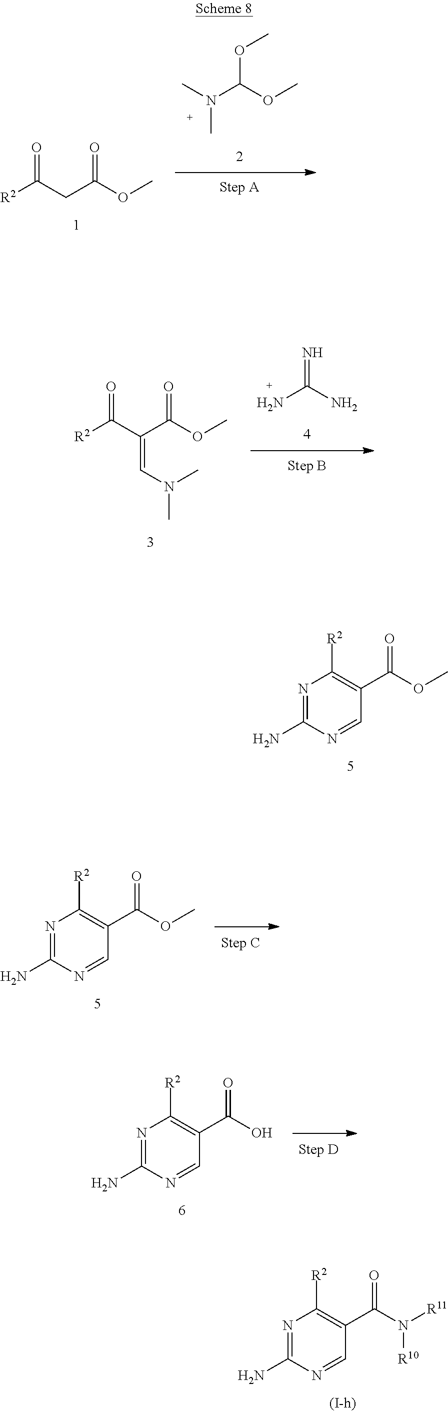

In scheme 8, R² is phenyl or phenylethyl; R¹⁰ is methyl; and R¹¹ is phenyl or phenylmethyl.

Step A: Enamines 3 can be obtained by condensation of beta-ketoesters 1 with 1,1-dimethoxy-N,N-dimethylmethanamine 2 in a solvent such as toluene or EtOH at 80-120° for 2-16 hrs.

Step B: Aminopyrimidines 5 can be obtained by condensation of enamines 3 with guanidine hydrochloride 4 in the presence of a base such as Na₂CO₃, NaOH, NaOMe, NaOEt, triethylamine in a solvent such as MeOH/water, EtOH, butanol at 50-120° C. for 2-16 hrs.

Step C: Acids 6 can be obtained by hydrolysis of esters 5 with a base such as NaOH or LiOH in a solvent such as THF, EtOH, Acetontrile, MeOH in the presence of a suitable amount of water at 0°-20° C. for 2-16 hrs.

Step D: Aminopyrimidine intermediates (I-h) can be obtained by reacting acid intermediates 6 with a suitable amine, using a coupling reagent such as DCC, HATU, EDCI or propyl phosphonic anhydride optionally in the presence of a base such as triethylamine, DIPEA or pyridine in a solvent such as dichloromethane, THF, acetonitrile, ethyl acetate or DMF at RT—80° C. for 2-12 hrs. The intermediates (I-h) can be further processed to a compound of formula (I) according to steps A and B of scheme 1.

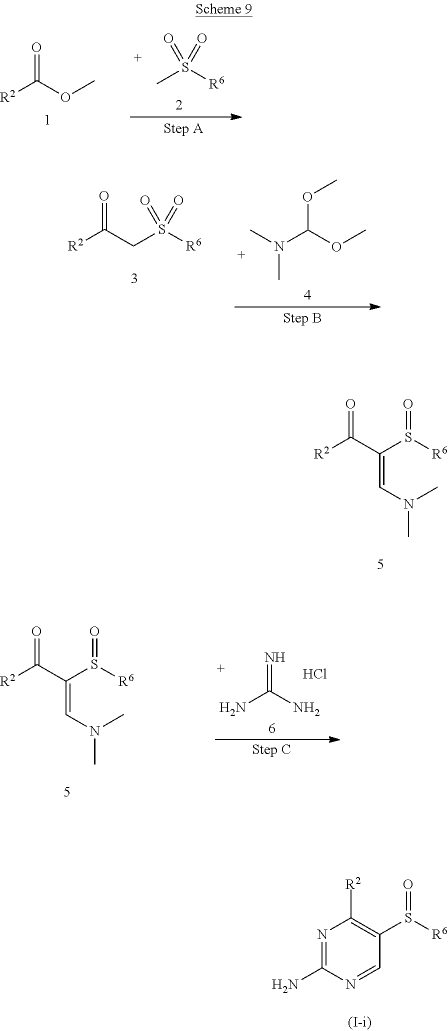

In scheme 9, R² is chlorophenyl, fluorophenyl or phenylethyl; R⁶ is methyl or phenyl.

Step A: beta-Ketosulfones 3 can be obtained by condensation of an ester 1 with a sulfone 2 in very dry DMSO, deprotonating the sulfone with a base such as sodium hydride first at between around 40-60° C. for 1-4 hrs, then reacting the ester at RT for between around 1-2 hrs.

Step B: Enamines 5 can be obtained by condensation of beta-ketosulfones 3 with 1,1-dimethoxy-N,N-dimethylmethanamine 4 in a solvent such as toluene or EtOH at between around 80-120° C. for 2-16 hrs.

Step C: Aminopyrimidine intermediates (I-i) can be obtained by condensation of enamines 5 with guanidine hydrochloride 6 in the presence of a base such as Na₂CO₃, NaOH, NaOMe, NaOEt, triethylamine in a solvent such as MeOH/water, EtOH, butanol at between around 50-120° C. for 2-16 hrs. The intermediates (I-i) can be further processed to a compound of formula (I) according to steps A and B of scheme 1.

The invention thus also relates to a process for the preparation of a compound according to the invention, comprising the coupling of a compound of formula (B1)

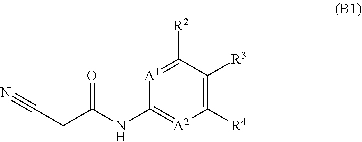

with a compound of formula (B2)

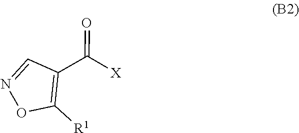

in the presence of a base;
wherein R¹-R⁴, A¹ and A² are as defined above; X is a leaving group such as halogen, mesylate or tosylate.

Conveniently X is a halogen, in particular chloride.

The coupling can conveniently be carried out in a solvent. The solvent can be for example THF, dichloromethane or a mixture thereof.

In the coupling the base can be for example NaH or tert-butoxide. Conveniently the base is NaH.

Convenient conditions for the coupling can be between around 0° C.-100° C., particularly between around 5° C.-80° C., more particularly between around 10° C.-50° C.

Preferred conditions for the coupling are the use of NaH in a mixture of THE and dichloromethane at around room temperature for between around 1-24 hrs, in particular between around 5-20 hrs.

The invention also relates to a compound according to the invention when manufactured according to a process of the invention.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compound of formula (I) is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal, epidural and intranasal, and if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The invention also relates in particular to:

A compound of formula (I) for use as therapeutically active substance;

A pharmaceutical composition comprising a compound of formula (I) and a therapeutically inert carrier;

A compound of formula (I) for use in the treatment of a disease modulated by cGAS;

The use of a compound of formula (I) for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS);

The use of a compound of formula (I) for the preparation of a medicament for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS);

A compound of formula (I) for use in the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS); and

A method for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS), which method comprises administering an effective amount of a compound of formula (I) to a patient in need thereof.

The invention will now be illustrated by the following examples which have no limiting character.

### EXAMPLES

### Abbreviations

AcOH=acetic acid; ATP=adenosine triphosphate; BINAP=(2,2′-bis(diphenylphosphino)-1,1′-binaphthyl); BSA=bovine serum albumine; DCC=N,N′-dicyclohexylcarbodiimide; DCM=dichloromethane; DIPEA=diisopropylethylamine; DMF=dimethylformamide; DMSO=diemethyl sulfoxide; DNA=deoxyribonucleic acid; EDC=ethylene dichloride; EDCI=1-ethyl-3-(3-dimethylaminopropyl)carbodiimide; ESI=electrospray ionization; EtOAc=ethyl acetate; EtOH=ethanol; GTP=guanosine triphosphate; HATU=hexafluorophosphate azabenzotriazole tetramethyl uronium; HPLC=high performance liquid chromatography; mCPBA=meta-chloroperoxybenzoic acid; MeOH=methanol; MS=mass spectrometry; NMP=N-methyl-2-pyrrolidone; RT=room temperature; SD=standard deviation; THE=tetrahydrofuran; TLC=thin-layer chromatography; TRIS=tris(hydroxymethyl)aminomethane.

### Example 1

### (Z)-2-cyano-N-(4-(N,N-dimethylsulfamoyl)phenyl)-3-hydroxy-3-(5-methylisoxazol-4-yl)acrylamide

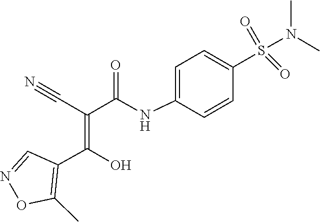 [SMILES: Cc1oncc1/C(O)=C(\C#N)C(=O)Nc1ccc(S(=O)(=O)N(C)C)cc1]

### Step 1:

To a stirred solution of 4-amino-N,N-dimethylbenzenesulfonamide (200 mg, 1 mmol), HATU (418 mg, 1.1 mmol, Eq: 1.1) and DIPEA (388 mg, 523 μl, 3 mmol, Eq: 3) at RT in DMF (6.24 ml) under an argon atmosphere was added 2-cyanoacetic acid (128 mg, 1.5 mmol, Eq: 1.5). Stirring at RT was continued over night.

The solution was diluted with EtOAc and washed with H₂O. The aqueous phase was back extracted with EtOAc. The combined organics were washed with H₂O and brine, dried (MgSO₄), filtered and concentrated to leave the crude product as a light yellow liquid.

The crude product (0.328 g) was purified by flash chromatography using heptane/EtOAc (20-100%) as eluent.

### Step 2:

To a stirred solution of 2-cyano-N-(4-(N,N-dimethylsulfamoyl)phenyl)acetamide (182 mg, 681 μmol) at RT in THE (3.9 ml) under an argon atmosphere was added sodium hydride 60% dispersion in mineral oil (62.6 mg, 1.57 mmol). After stirring for 10 min, a solution of 5-methylisoxazole-4-carbonyl chloride (107 mg, 715 μmol) in CH₂Cl₂ (388 μl) was added in one portion. Stirring at RT was continued for 17 hrs. The mixture was carefully treated with 0.5M HCl (3 ml), diluted with brine and extracted with DCM. The combined organics were, dried (MgSO₄), filtered and concentrated to leave the crude product as a brown/orange sticky solid. The crude product (0.26 g) was triturated in 2 ml MeOH, stirrred for 15 minutes, filtered, washed with Et₂O and dried to give the title compound.

In analogy to the procedures described in example 1, examples 2-109 were prepared starting from suitable aniline, aminopyridine or aminopyrimidine starting materials (table 1).

TABLE 1

| Ex. | Systematic name | Structure | MS result |
|---|---|---|---|
| 2 | (Z)-N-(4-acetylphenyl)-2- cyano-3-hydroxy-3-(5- methylisoxazol-4-yl)prop- 2-enamide | 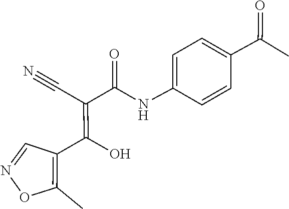 [SMILES: CC(=O)c1ccc(NC(=O)/C(C#N)=C(\O)c2cnoc2C)cc1] | 310.1 [M − H]− ESI neg. |
| 3 | (Z)-2-cyano-3-hydroxy-3- (5-methylisoxazol-4-yl)- N-phenylacrylamide | 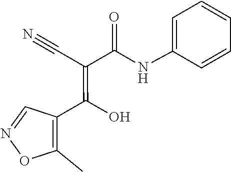 [SMILES: Cc1oncc1/C(O)=C(\C#N)C(=O)Nc1ccccc1] | 270.2 [M + H]+ ESI pos. |
| 4 | (Z)-2-cyano-3-hydroxy-3- (5-methylisoxazol-4-yl)- N-(4- (trifluoromethyl)phenyl) acrylamide | 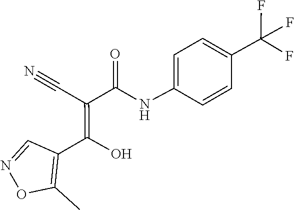 [SMILES: Cc1oncc1/C(O)=C(\C#N)C(=O)Nc1ccc(C(F)(F)F)cc1] | 338.1 [M + H]+ ESI pos. |
| 5 | (Z)-2-cyano-3-hydroxy-3- (5-methylisoxazol-4-yl)- N-[3- (trifluoromethyl)phenyl] prop-2-enamide | 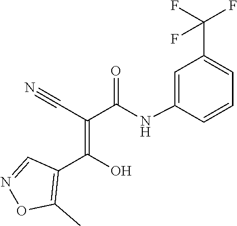 [SMILES: Cc1oncc1/C(O)=C(\C#N)C(=O)Nc1cccc(C(F)(F)F)c1] | n.a. |
| 6 | (Z)-N-(3,5- bis(trifluoromethyl)phenyl)- 2-cyano-3-hydroxy-3-(5- methylisoxazol-4- yl)acrylamide | 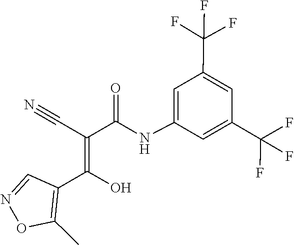 [SMILES: Cc1oncc1/C(O)=C(\C#N)C(=O)Nc1cc(C(F)(F)F)cc(C(F)(F)F)c1] | 404.2 [M − H]− ESI neg. |
| 7 | (Z)-2-cyano-N-(4- cyanophenyl)-3-hydroxy- 3-(5-methylisoxazol-4- yl)acrylamide | 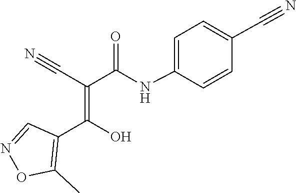 [SMILES: Cc1oncc1/C(O)=C(\C#N)C(=O)Nc1ccc(C#N)cc1] | 295.1 [M + H]+ ESI pos. |
| 8 | methyl (Z)-4-(2-cyano-3- hydroxy-3-(5- methylisoxazol-4- yl)acrylamido)benzoate | 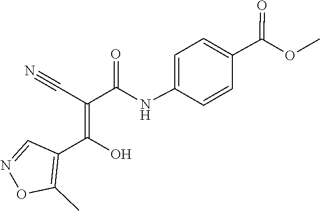 [SMILES: COC(=O)c1ccc(NC(=O)/C(C#N)=C(\O)c2cnoc2C)cc1] | 328.1 [M + H]+ ESI pos. |
| 9 | (Z)-2-cyano-3-hydroxy-3- (5-methylisoxazol-4-yl)- N-(2- (trifluoromethyl)phenyl) acrylamide | 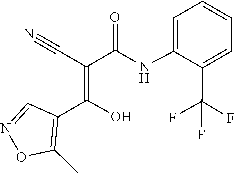 [SMILES: Cc1oncc1/C(O)=C(\C#N)C(=O)Nc1ccccc1C(F)(F)F] | 338.1 [M + H]+ ESI pos. |
| 10 | (Z)-2-cyano-N-(4- fluorophenyl)-3-hydroxy- 3-(5-methylisoxazol-4- yl)acrylamide | 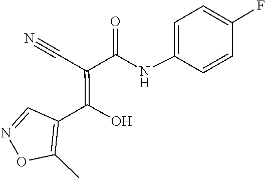 [SMILES: Cc1oncc1/C(O)=C(\C#N)C(=O)Nc1ccc(F)cc1] | 288.1 [M + H]+ ESI pos. |
| 11 | (Z)-2-cyano-3-hydroxy-3- (5-methylisoxazol-4-yl)- N-(4- (trifluoromethoxy)phenyl) acrylamide | 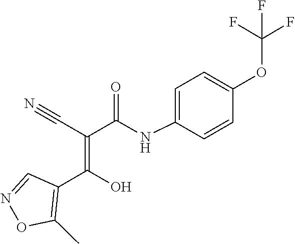 [SMILES: Cc1oncc1/C(O)=C(\C#N)C(=O)Nc1ccc(OC(F)(F)F)cc1] | 354.1 [M + H]+ ESI pos. |
| 12 | (Z)-2-cyano-N-(3,4- dichlorophenyl)-3- hydroxy-3-(5- methylisoxazol-4- yl)acrylamide | 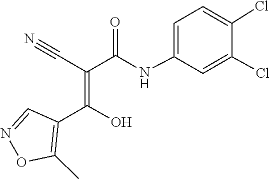 [SMILES: Cc1oncc1/C(O)=C(\C#N)C(=O)Nc1ccc(Cl)c(Cl)c1] | 338.0 [M + H]+ ESI pos. |
| 13 | (Z)-N-(4-((4- chlorophenyl)thio)phenyl)- 2-cyano-3-hydroxy-3-(5- methylisoxazol-4- yl)acrylamide | 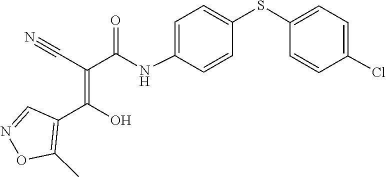 [SMILES: Cc1oncc1/C(O)=C(\C#N)C(=O)Nc1ccc(Sc2ccc(Cl)cc2)cc1] | 410.2 [M − H]− ESI neg. |
| 14 | (Z)-2-cyano-3-hydroxy-3- (5-methylisoxazol-4-yl)- N-(4- (methylthio)phenyl) acrylamide | 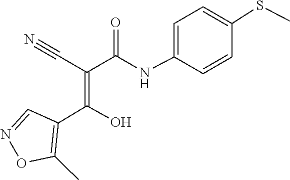 [SMILES: CSc1ccc(NC(=O)/C(C#N)=C(\O)c2cnoc2C)cc1] | 316.1 [M + H]+ ESI pos. |
| 15 | (Z)-N-(4-chloro-3- (trifluoromethyl)phenyl)- 2-cyano-3-hydroxy-3-(5- methylisoxazol-4- yl)acrylamide | 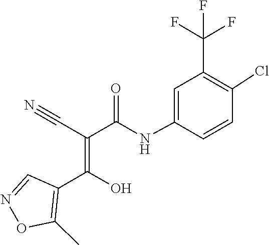 [SMILES: Cc1oncc1/C(O)=C(\C#N)C(=O)Nc1ccc(Cl)c(C(F)(F)F)c1] | 372.1 [M + H]+ ESI pos. |
| 16 | (Z)-N-(4-((4- chlorophenyl)sulfonyl) phenyl)-2-cyano-3-hydroxy-3- (5-methylisoxazol-4- yl)acrylamide | 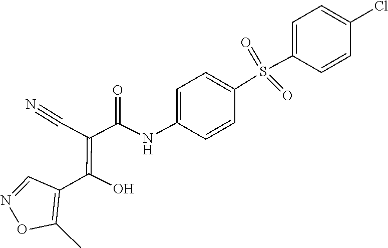 [SMILES: Cc1oncc1/C(O)=C(\C#N)C(=O)Nc1ccc(S(=O)(=O)c2ccc(Cl)cc2)cc1] | 444.2 [M + H]+ ESI pos. |
| 17 | (Z)-2-cyano-3-hydroxy-N- (4-methoxyphenyl)-3-(5- methylisoxazol-4- yl)acrylamide | 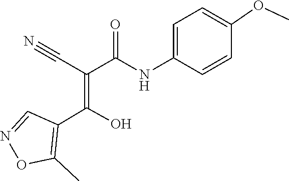 [SMILES: COc1ccc(NC(=O)/C(C#N)=C(\O)c2cnoc2C)cc1] | 300.1 [M + H]+ ESI pos. |
| 18 | (Z)-2-cyano-3-hydroxy-3- (5-methylisoxazol-4-yl)- N-(4-(3- (trifluoromethyl)phenoxy) phenyl)acrylamide | 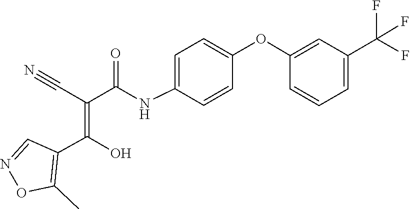 [SMILES: Cc1oncc1/C(O)=C(\C#N)C(=O)Nc1ccc(Oc2cccc(C(F)(F)F)c2)cc1] | 430.2 [M + H]+ ESI pos. |
| 19 | (Z)-2-cyano-3-hydroxy-N- (3-methyl-4-(4- (trifluoromethyl)phenoxy) phenyl)-3-(5- methylisoxazol-4- yl)acrylamide | 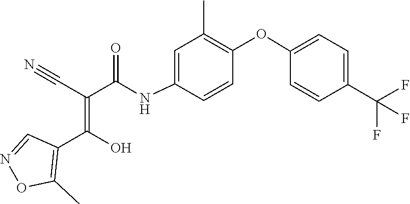 [SMILES: Cc1cc(NC(=O)/C(C#N)=C(\O)c2cnoc2C)ccc1Oc1ccc(C(F)(F)F)cc1] | 444.2 [M + H]+ ESI pos. |
| 20 | (Z)-N-(4-chlorophenyl)-2- cyano-3-hydroxy-3-(5- methylisoxazol-4- yl)acrylamide | 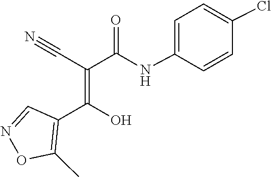 [SMILES: Cc1oncc1/C(O)=C(\C#N)C(=O)Nc1ccc(Cl)cc1] | 304.1 [M + H]+ ESI pos. |
| 21 | (Z)-N-(4-(1H-pyrrol-1- yl)phenyl)-2-cyano-3- hydroxy-3-(5- methylisoxazol-4- yl)acrylamide | 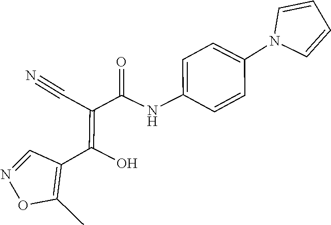 [SMILES: Cc1oncc1/C(O)=C(\C#N)C(=O)Nc1ccc(-n2cccc2)cc1] | 333.3 [M − H]− ESI neg. |
| 22 | (Z)-2-cyano-N-(4-cyano- 2-methylphenyl)-3- hydroxy-3-(5- methylisoxazol-4- yl)acrylamide | 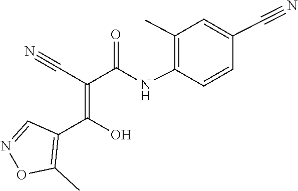 [SMILES: Cc1cc(C#N)ccc1NC(=O)/C(C#N)=C(\O)c1cnoc1C] | 309.2 [M + H]+ ESI pos. |
| 23 | (Z)-N-(4-bromo-3- methylphenyl)-2-cyano-3- hydroxy-3-(5- methylisoxazol-4- yl)acrylamide | 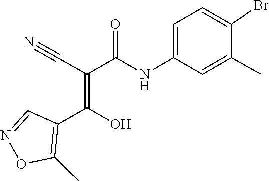 [SMILES: Cc1cc(NC(=O)/C(C#N)=C(\O)c2cnoc2C)ccc1Br] | 364.1 [M + H]+ ESI pos. |
| 24 | (Z)-N-(3-chloro-4-(4- chlorophenoxy)phenyl)-2- cyano-2-hydroxy-3-(5- methylisoxazol-4- yl)acrylamide | 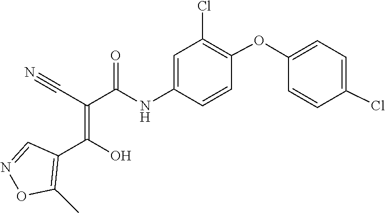 [SMILES: Cc1oncc1/C(O)=C(\C#N)C(=O)Nc1ccc(Oc2ccc(Cl)cc2)c(Cl)c1] | 428.2 [M − H]− ESI neg. |
| 25 | methyl 3-[[(Z)-2-cyano-3- hydroxy-3-(5- methylisoxazol-4-yl) prop-2-enoyl]amino]benzoate | 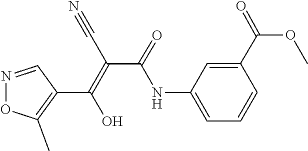 [SMILES: COC(=O)c1cccc(NC(=O)/C(C#N)=C(\O)c2cnoc2C)c1] | 328.1 [M + H]+ |
| 26 | (Z)-2-cyano-3-hydroxy-3- (5-methylisoxazol-4-yl)- N-(3- (methylsulfonyl)phenyl) acrylamide | 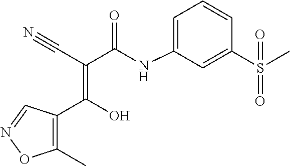 [SMILES: Cc1oncc1/C(O)=C(\C#N)C(=O)Nc1cccc(S(C)(=O)=O)c1] | 348.1 [M + H]+ ESI pos |
| 27 | (Z)-2-cyano-3-hydroxy-3- (5-methylisoxazol-4-yl)- N-(4-(pyrrolidin-1- ylsulfonyl)phenyl) acrylamide | 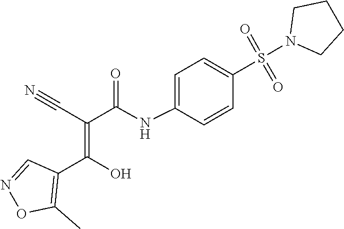 [SMILES: Cc1oncc1/C(O)=C(\C#N)C(=O)Nc1ccc(S(=O)(=O)N2CCCC2)cc1] | 403.2 [M + H]+ ESI pos |
| 28 | (Z)-2-cyano-N-(4- ethoxyphenyl)-3-hydroxy- 3-(5-methylisoxazol-4- yl)prop-2-enamide | 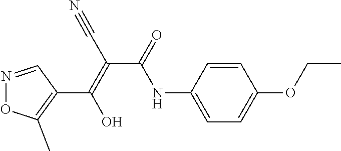 [SMILES: CCOc1ccc(NC(=O)/C(C#N)=C(\O)c2cnoc2C)cc1] | 314.1 [M + H]+ |
| 29 | (Z)-2-cyano-3-hydroxy-N- (4-(N-(2- methoxyethyl)sulfamoyl) phenyl)-3-(5- methylisoxazol-4- yl)acrylamide | 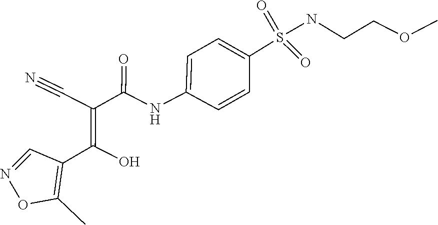 | 407.2 [M + H]+ ESI pos |
| 30 | (Z)-2-cyano-3-hydroxy-3- (5-methylisoxazol-4-yl)- N-(4- (phenylsulfonyl)phenyl) acrylamide | 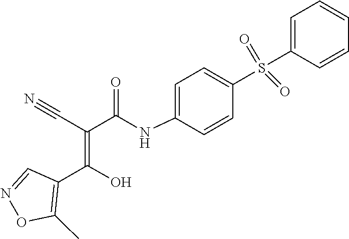 [SMILES: Cc1oncc1/C(O)=C(\C#N)C(=O)Nc1ccc(S(=O)(=O)c2ccccc2)cc1] | 410.2 [M + H]+ ESI pos |
| 31 | (Z)-2-cyano-3-hydroxy-N- (4-methoxy-3-methyl- phenyl)-3-(5- methylisoxazol-4-yl)prop- 2-enamide | 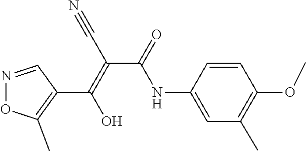 [SMILES: COc1ccc(NC(=O)/C(C#N)=C(\O)c2cnoc2C)cc1C] | 314.1 [M + H]+ |
| 32 | methyl 2-[4-[[(Z)-2-cyano- 3-hydroxy-3-(5- methylisoxazol-4-yl)prop- 2-enoyl]amino]phenyl] acetate | 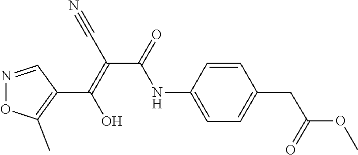 [SMILES: COC(=O)Cc1ccc(NC(=O)/C(C#N)=C(\O)c2cnoc2C)cc1] | 364.1 [M + Na]+ |
| 33 | (Z)-2-cyano-N-(3- cyanophenyl)-3-hydroxy- 3-(5-methylisoxazol-4- yl)prop-2-enamide | 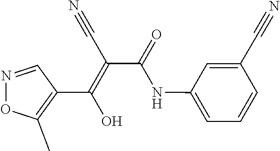 [SMILES: Cc1oncc1/C(O)=C(\C#N)C(=O)Nc1cccc(C#N)c1] | 295.1 [M + H]+ |
| 34 | (Z)-2-cyano-3-hydroxy-3- (5-methylisoxazol-4-yl)- N-(4- (phenylsulfinyl)phenyl) acrylamide | 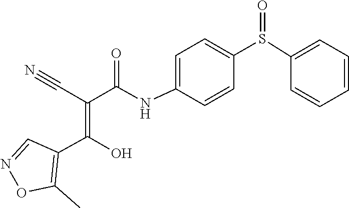 [SMILES: Cc1oncc1/C(O)=C(\C#N)C(=O)Nc1ccc(S(=O)c2ccccc2)cc1] | 394.2 [M + H]+ ESI pos |
| 35 | 4-[[(Z)-2-cyano-3- hydroxy-3-(5- methylisoxazol-4-yl)prop- 2-enoyl]amino]-N,N- dimethyl-benzamide | 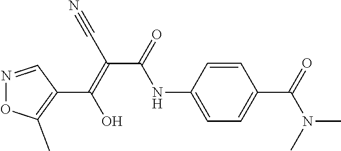 [SMILES: Cc1oncc1/C(O)=C(\C#N)C(=O)Nc1ccc(C(=O)N(C)C)cc1] | 341.1 [M + H]+ |
| 36 | 4-[[(Z)-2-cyano-3- hydroxy-3-(5- methylisoxazol-4-yl)prop- 2-enoyl]amino]-N-methyl- benzamide | 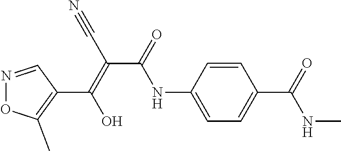 [SMILES: CNC(=O)c1ccc(NC(=O)/C(C#N)=C(\O)c2cnoc2C)cc1] | 327.1 [M + H]+ |
| 37 | (Z)-2-cyano-3-hydroxy-N- (5-methoxy-2-pyridyl)-3- (5-methylisoxazol-4- yl)prop-2-enamide | 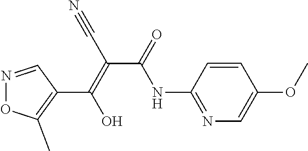 [SMILES: COc1ccc(NC(=O)/C(C#N)=C(\O)c2cnoc2C)nc1] | 301.1 [M + H]+ |
| 38 | (Z)-N-(4-benzoylphenyl)- 2-cyano-3-hydroxy-3-(5- methylisoxazol-4-yl)prop- 2-enamide | 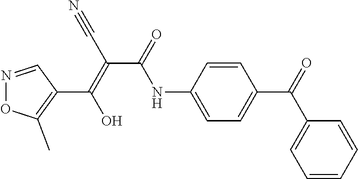 [SMILES: Cc1oncc1/C(O)=C(\C#N)C(=O)Nc1ccc(C(=O)c2ccccc2)cc1] | 374.1 [M + H]+ |
| 39 | (Z)-2-cyano-3-hydroxy-N- (4-((4- methoxyphenyl)sulfonyl) phenyl)-3-(5- methylisoxazol-4- yl)acrylamide | 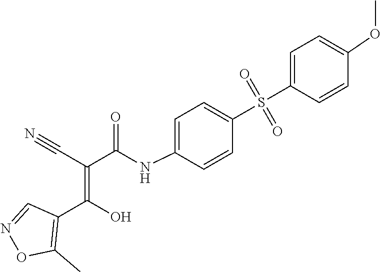 [SMILES: COc1ccc(S(=O)(=O)c2ccc(NC(=O)/C(C#N)=C(\O)c3cnoc3C)cc2)cc1] | 440.2 [M + H]+ ESI pos. |
| 40 | (Z)-2-cyano-3-hydroxy-3- (5-methylisoxazol-4-yl)- N-[4-(thiazol-2- ylsulfamoyl)phenyl]prop- 2-enamide | 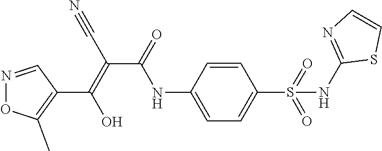 [SMILES: Cc1oncc1/C(O)=C(\C#N)C(=O)Nc1ccc(S(=O)(=O)Nc2nccs2)cc1] | 432.1 [M + H]+ |
| 41 | (Z)-2-cyano-3-hydroxy-3- (5-methylisoxazol-4-yl)- N-[4-(oxetan-3- yloxy)phenyl]prop-2- enamide | 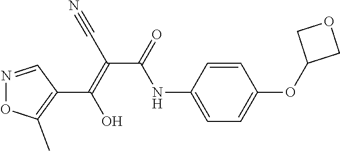 [SMILES: Cc1oncc1/C(O)=C(\C#N)C(=O)Nc1ccc(OC2COC2)cc1] | 342.1 [M + H]+ |
| 42 | (Z)-N-(4- acetamidophenyl)-2- cyano-3-hydroxy-3-(5- methylisoxazol-4-yl)prop- 2-enamide | 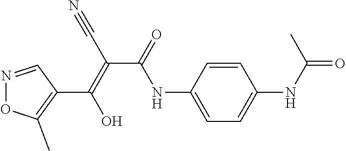 [SMILES: CC(=O)Nc1ccc(NC(=O)/C(C#N)=C(\O)c2cnoc2C)cc1] | 327.1 [M + H]+ |
| 43 | (Z)-2-cyano-3-hydroxy-3- (5-methylisoxazol-4-yl)- N-[4-[(5-methyl-1,3,4- thiadiazol-2- yl)sulfamoyl]phenyl]prop- 2-enamide | 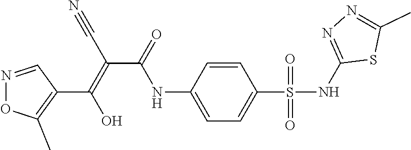 [SMILES: Cc1nnc(NS(=O)(=O)c2ccc(NC(=O)/C(C#N)=C(\O)c3cnoc3C)cc2)s1] | 447.1 [M + H]+ |
| 44 | 4-[[(Z)-2-cyano-3- hydroxy-3-(5- methylisoxazol-4-yl)prop- 2-enoyl]amino]benzamide | 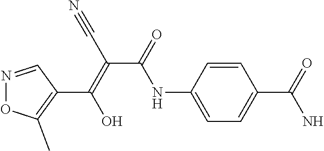 [SMILES: Cc1oncc1/C(O)=C(\C#N)C(=O)Nc1ccc(C(N)=O)cc1] | 313.1 [M + H]+ |
| 45 | (Z)-2-cyano-3-hydroxy-3- (5-methylisoxazol-4-yl)- N-[4-(pyrimidin-2- ylsulfamoyl)phenyl]prop- 2-enamide | 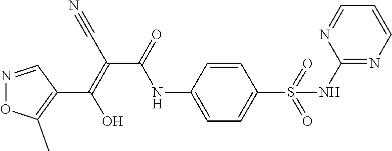 [SMILES: Cc1oncc1/C(O)=C(\C#N)C(=O)Nc1ccc(S(=O)(=O)Nc2ncccn2)cc1] | 427.1 [M + H]+ |
| 46 | (Z)-2-cyano-3-hydroxy-N- (5-methoxypyrimidin-2- yl)-3-(5-methylisoxazol-4- yl)prop-2-enamide | 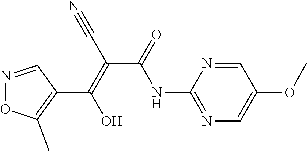 [SMILES: COc1cnc(NC(=O)/C(C#N)=C(\O)c2cnoc2C)nc1] | 301.8 [M + H]+ |
| 47 | (Z)-2-cyano-3-hydroxy-3- (5-methylisoxazol-4-yl)- N-(4- (morpholinosulfonyl) phenyl)acrylamide | 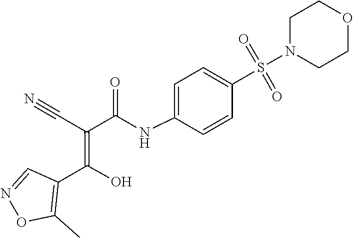 [SMILES: Cc1oncc1/C(O)=C(\C#N)C(=O)Nc1ccc(S(=O)(=O)N2CCOCC2)cc1] | 419.2 [M + H]+ ESI pos. |
| 48 | (Z)-2-cyano-3-hydroxy-3- (5-methylisoxazol-4-yl)- N-(4-(N- propylsulfamoyl)phenyl) acrylamide | 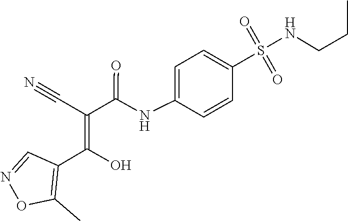 [SMILES: CCCNS(=O)(=O)c1ccc(NC(=O)/C(C#N)=C(\O)c2cnoc2C)cc1] | 391.2 [M + H]+ ESI pos. |
| 49 | (Z)-2-cyano-3-hydroxy-3- (5-methylisoxazol-4-yl)- N-(3- (phenylsulfonyl)phenyl) acrylamide | 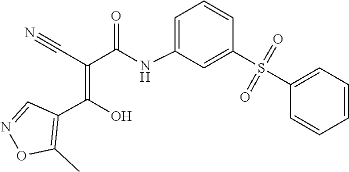 [SMILES: Cc1oncc1/C(O)=C(\C#N)C(=O)Nc1cccc(S(=O)(=O)c2ccccc2)c1] | 410.2 [M + H]+ ESI pos. |
| 50 | (Z)-2-cyano-3-hydroxy-N- (2-methyl-4- (trifluoromethyl)phenyl)- 3-(5-methylisoxazol-4- yl)acrylamide | 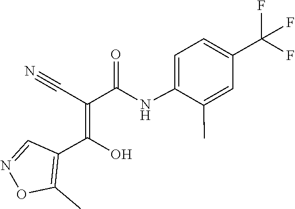 [SMILES: Cc1cc(C(F)(F)F)ccc1NC(=O)/C(C#N)=C(\O)c1cnoc1C] | 350.1 [M − H]− ESI neg. |
| 51 | (Z)-2-cyano-3-hydroxy-3- (5-methylisoxazol-4-yl)- N-(3-phenylphenyl) prop-2-enamide | 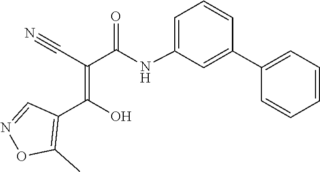 [SMILES: Cc1oncc1/C(O)=C(\C#N)C(=O)Nc1cccc(-c2ccccc2)c1] | 346.2 [M + H]+ ESI pos. |
| 52 | (Z)-2-cyano-3-hydroxy-3- (5-methylisoxazol-4-yl)- N-(3- phenoxyphenyl)acrylamide | 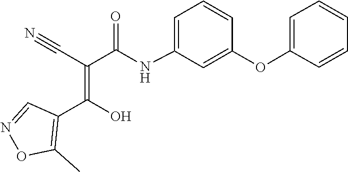 [SMILES: Cc1oncc1/C(O)=C(\C#N)C(=O)Nc1cccc(Oc2ccccc2)c1] | 362.2 [M + H]+ ESI pos. |
| 53 | (Z)-2-cyano-N-(4-(N-(2- (diethylamino)-2- oxoethyl)sulfamoyl)phenyl)- 3-hydroxy-3-(5- methylisoxazol-4- yl)acrylamide | 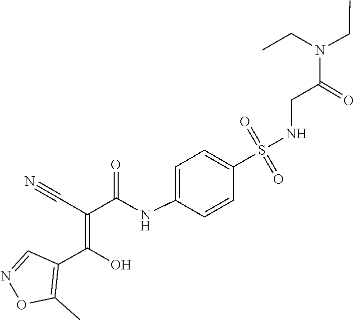 [SMILES: CCN(CC)C(=O)CNS(=O)(=O)c1ccc(NC(=O)/C(C#N)=C(\O)c2cnoc2C)cc1] | 462.2 [M + H]+ ESI pos. |
| 54 | (Z)-2-cyano-3-hydroxy-3- (5-methylisoxazol-4-yl)- N-(4-(N- phenylsulfamoyl)phenyl) acrylamide | 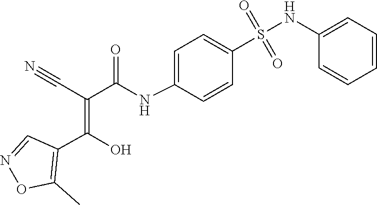 [SMILES: Cc1oncc1/C(O)=C(\C#N)C(=O)Nc1ccc(S(=O)(=O)Nc2ccccc2)cc1] | 425.2 [M + H]+ ESI pos. |
| 55 | (Z)-2-cyano-3-hydroxy-3- (5-methylisoxazol-4-yl)- N-(4- (phenylthio)phenyl) acrylamide | 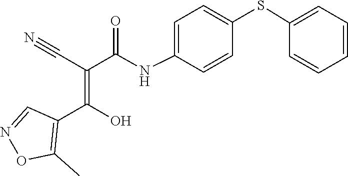 [SMILES: Cc1oncc1/C(O)=C(\C#N)C(=O)Nc1ccc(Sc2ccccc2)cc1] | 378.2 [M + H]+ ESI pos. |
| 56 | (Z)-N-(2-chloro-4- (trifluoromethyl)phenyl)- 2-cyano-3-hydroxy-3-(5- methylisoxazol-4- yl)acrylamide | 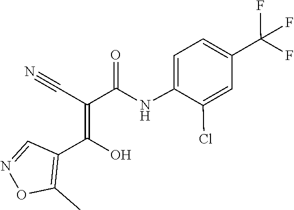 [SMILES: Cc1oncc1/C(O)=C(\C#N)C(=O)Nc1ccc(C(F)(F)F)cc1Cl] | 370.1 [M − H]− ESI neg. |
| 57 | (Z)-2-cyano-N-(4-(N-(4- cyanophenyl)sulfamoyl) phenyl)-3-hydroxy-3-(5- methylisoxazol-4- yl)acrylamide | 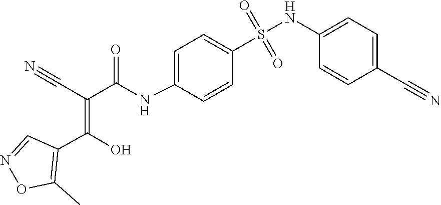 [SMILES: Cc1oncc1/C(O)=C(\C#N)C(=O)Nc1ccc(S(=O)(=O)Nc2ccc(C#N)cc2)cc1] | 448.0 [M − H]− ESI neg. |
| 58 | (Z)-N-((4-(2-cyano-3- hydroxy-3-(5- methylisoxazol-4- yl)acrylamido)phenyl) sulfonyl)-N-(4-cyanophenyl)- 5-methylisoxazole-4- carboxamide | 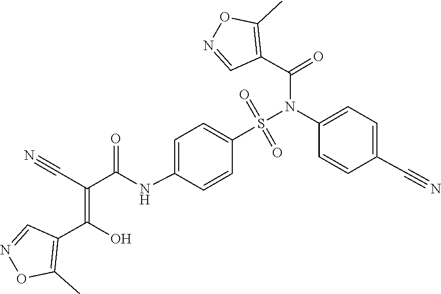 [SMILES: Cc1oncc1C(=O)N(c1ccc(C#N)cc1)S(=O)(=O)c1ccc(NC(=O)/C(C#N)=C(\O)c2cnoc2C)cc1] | 557.0 [M − H]− ESI pos. |
| 59 | (Z)-2-cyano-N-(4-(N-(3,5- dimethylisoxazol-4- yl)sulfamoyl)phenyl)-3- hydroxy-3-(5- methylisoxazol-4- yl)acrylamide | 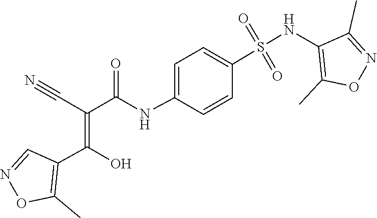 [SMILES: Cc1noc(C)c1NS(=O)(=O)c1ccc(NC(=O)/C(C#N)=C(\O)c2cnoc2C)cc1] | 444.2 [M + H]+ ESI pos. |
| 60 | (Z)-N-((4-(2-cyano-3- hydroxy-3-(5- methylisoxazol-4- yl)acrylamido)phenyl) sulfamoyl)-N-(3,5- dimethylisoxazol-4-yl)-5- methylisoxazole-4- carboxamide | 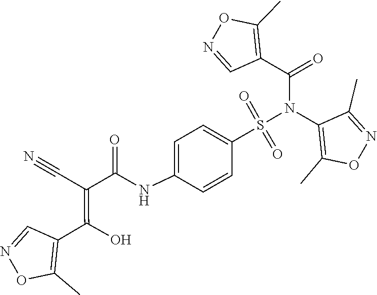 [SMILES: Cc1noc(C)c1N(C(=O)c1cnoc1C)S(=O)(=O)c1ccc(NC(=O)/C(C#N)=C(\O)c2cnoc2C)cc1] | 553.2 [M + H]+ ESI pos |
| 61 | (Z)-N-(2-chloro-4- methylsulfonyl-phenyl)-2- cyano-3-hydroxy-3-(5- methylisoxazol-4-yl)prop- 2-enamide | 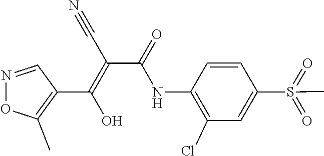 [SMILES: Cc1oncc1/C(O)=C(\C#N)C(=O)Nc1ccc(S(C)(=O)=O)cc1Cl] | 404.0 [M + Na]+ |
| 62 | (Z)-2-cyano-3-hydroxy-3- (5-methylisoxazol-4-yl)- N-(4- phenoxyphenyl)acrylamide | 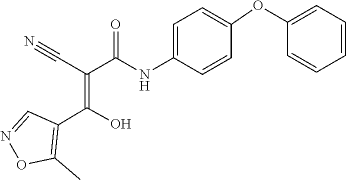 [SMILES: Cc1oncc1/C(O)=C(\C#N)C(=O)Nc1ccc(Oc2ccccc2)cc1] | 362.2 [M + H]+ ESI pos. |
| 63 | (Z)-N-(4-(4- chlorophenoxy)phenyl)-2- cyano-3-hydroxy-3-(5- methylisoxazol-4- yl)acrylamide | 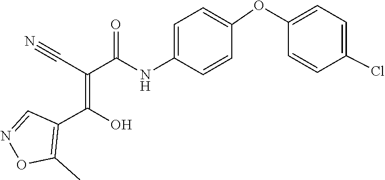 [SMILES: Cc1oncc1/C(O)=C(\C#N)C(=O)Nc1ccc(Oc2ccc(Cl)cc2)cc1] | 394.2 [M − H]− ESI neg. |
| 64 | (Z)-2-cyano-3-hydroxy-3- (5-methylisoxazol-4-yl)- N-(4-phenylphenyl)- prop-2-enamide | 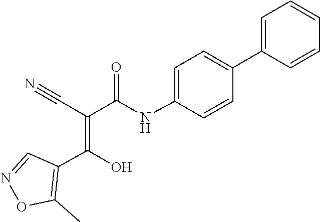 [SMILES: Cc1oncc1/C(O)=C(\C#N)C(=O)Nc1ccc(-c2ccccc2)cc1] | 346.1 [M + H]+ ESI pos. |
| 65 | (Z)-N-(3-chloro-4- (trifluoromethyl)phenyl)- 2-cyano-3-hydroxy-3-(5- methylisoxazol-4- yl)acrylamide | 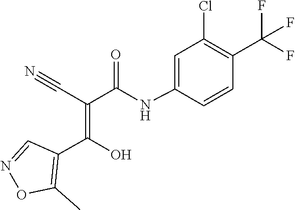 [SMILES: Cc1oncc1/C(O)=C(\C#N)C(=O)Nc1ccc(C(F)(F)F)c(Cl)c1] | 370.1 [M − H]− ESI neg. |
| 66 | (Z)-2-cyano-3-hydroxy-3- (5-methylisoxazol-4-yl)- N-(5- (trifluoromethyl)pyridin-2- yl)acrylamide | 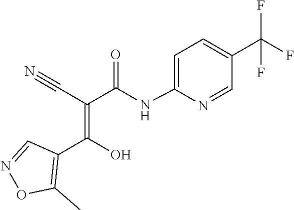 [SMILES: Cc1oncc1/C(O)=C(\C#N)C(=O)Nc1ccc(C(F)(F)F)cn1] | 339.1 [M + H]+ ESI pos. |
| 67 | (Z)-4-(2-cyano-3-hydroxy- 3-(5-methylisoxazol-4- yl)acrylamido)-N- phenylbenzamide | 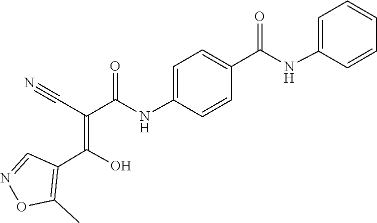 [SMILES: Cc1oncc1/C(O)=C(\C#N)C(=O)Nc1ccc(C(=O)Nc2ccccc2)cc1] | 389.2 [M + H]+ ESI pos. |
| 68 | (Z)-2-cyano-3-hydroxy-3- (5-methylisoxazol-4-yl)- N-(4-phenylpyrimidin-2- yl)acrylamide | 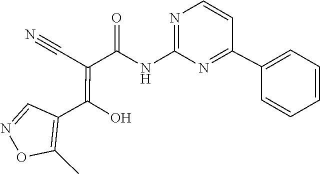 [SMILES: Cc1oncc1/C(O)=C(\C#N)C(=O)Nc1nccc(-c2ccccc2)n1] | 348.1 [M + H]+ ESI pos. |
| 69 | (Z)-3-(2-cyano-3-hydroxy- 3-(5-methylisoxazol-4- yl)acrylamido)-N- phenylbenzamide | 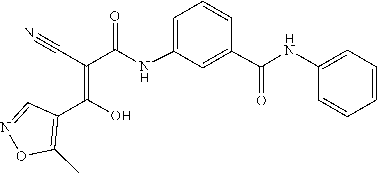 [SMILES: Cc1oncc1/C(O)=C(\C#N)C(=O)Nc1cccc(C(=O)Nc2ccccc2)c1] | 389.1 [M + H]+ ESI pos. |
| 70 | (S,Z)-N-(1- benzylpyrrolidin-3-yl)-4- (2-cyano-3-hydroxy-3-(5- methylisoxazol-4- yl)acrylamido)benzamide | 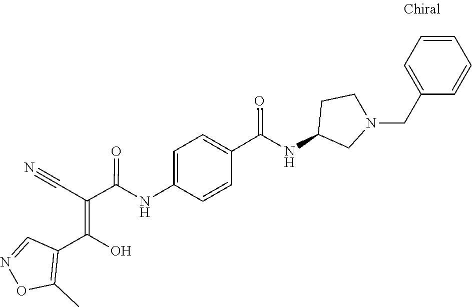 [SMILES: Cc1oncc1/C(O)=C(\C#N)C(=O)Nc1ccc(C(=O)N[C@H]2CCN(Cc3ccccc3)C2)cc1] | 472.2 [M + H]+ ESI pos. |
| 71 | (Z)-N-(3-(2-cyano-3- hydroxy-3-(5- methylisoxazol-4- yl)acrylamido)phenyl) benzamide | 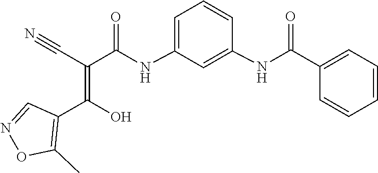 [SMILES: Cc1oncc1/C(O)=C(\C#N)C(=O)Nc1cccc(NC(=O)c2ccccc2)c1] | 389.2 [M + H]+ ESI pos. |
| 72 | (Z)-N-benzyl-4-(2-cyano- 3-hydroxy-3-(5- methylisoxazol-4- yl)acrylamido)benzamide | 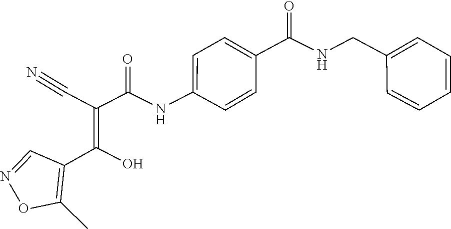 [SMILES: Cc1oncc1/C(O)=C(\C#N)C(=O)Nc1ccc(C(=O)NCc2ccccc2)cc1] | 403.1 [M + H]+ ESI pos. |
| 73 | (Z)-2-cyano-N-(5- (cyclohexyloxy)pyrimidin- 2-yl)-3-hydroxy-3-(5- methylisoxazol-4- yl)acrylamide | 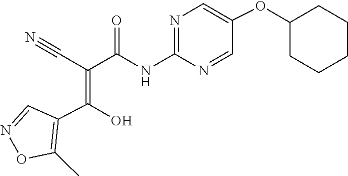 [SMILES: Cc1oncc1/C(O)=C(\C#N)C(=O)Nc1ncc(OC2CCCCC2)cn1] | 370.2 [M + H]+ ESI pos. |
| 74 | (Z)-N-(4-benzylphenyl)-2- cyano-3-hydroxy-3-(5- methylisoxazol-4- yl)acrylamide | 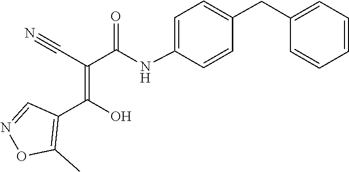 [SMILES: Cc1oncc1/C(O)=C(\C#N)C(=O)Nc1ccc(Cc2ccccc2)cc1] | 360.2 [M + H]+ ESI pos. |
| 75 | (Z)-2-cyano-3-hydroxy-N- (3-methyl-4- (trifluoromethyl)phenyl)- 3-(5-methylisoxazol-4- yl)acrylamide | 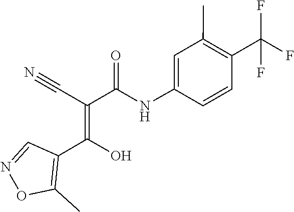 [SMILES: Cc1cc(NC(=O)/C(C#N)=C(\O)c2cnoc2C)ccc1C(F)(F)F] | 350.1 [M − H]− ESI neg. |
| 76 | (Z)-2-cyano-3-hydroxy-N- (3-methoxy-4- (trifluoromethyl)phenyl)- 3-(5-methylisoxazol-4- yl)acrylamide | 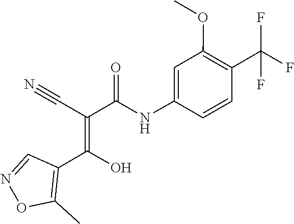 [SMILES: COc1cc(NC(=O)/C(C#N)=C(\O)c2cnoc2C)ccc1C(F)(F)F] | 366.1 [M − H]− ESI neg. |
| 77 | (Z)-2-chloro-4-(2-cyano-3- hydroxy-3-(5- methylisoxazol-4- yl)acrylamido)-N- (cyclohexylmethyl)-N- methylbenzamide | 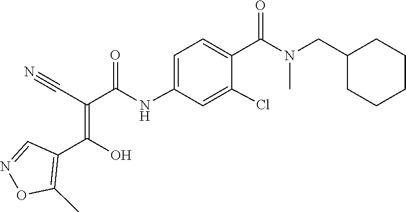 [SMILES: Cc1oncc1/C(O)=C(\C#N)C(=O)Nc1ccc(C(=O)N(C)CC2CCCCC2)c(Cl)c1] | 457.2 [M + H]+ ESI pos. |
| 78 | (Z)-2-cyano-N-(3-cyano- 4- (trifluoromethyl)phenyl)- 3-hydroxy-3-(5- methylisoxazol-4- yl)acrylamide | 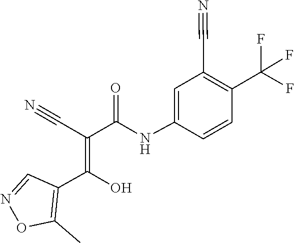 [SMILES: Cc1oncc1/C(O)=C(\C#N)C(=O)Nc1ccc(C(F)(F)F)c(C#N)c1] | 361.1 [M − H]− ESI neg. |
| 79 | (Z)-2-cyano-N-(5- cyclohexylpyrimidin-2- yl)-3-hydroxy-3-(5- methylisoxazol-4- yl)acrylamide | 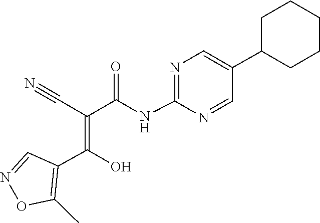 [SMILES: Cc1oncc1/C(O)=C(\C#N)C(=O)Nc1ncc(C2CCCCC2)cn1] | 354.2 [M + H]+ ESI pos. |
| 80 | (Z)-2-cyano-3-hydroxy-N- (5-(4- methoxyphenyl)pyrimidin- 2-yl)-3-(5-methylisoxazol- 4-yl)acrylamide | 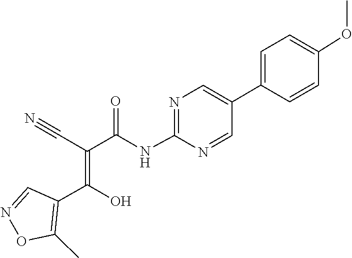 [SMILES: COc1ccc(-c2cnc(NC(=O)/C(C#N)=C(\O)c3cnoc3C)nc2)cc1] | 378.2 [M + H]+ ESI pos. |
| 81 | (Z)-2-cyano-3-hydroxy-3- (5-methylisoxazol-4-yl)- N-(5-phenylpyrimidin-2- yl)acrylamide | 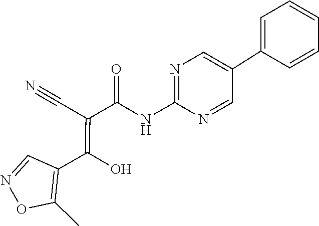 [SMILES: Cc1oncc1/C(O)=C(\C#N)C(=O)Nc1ncc(-c2ccccc2)cn1] | 348.1 [M + H]+ ESI pos. |
| 82 | (Z)-2-cyano-3-hydroxy-3- (5-methylisoxazol-4-yl)- N-(5-phenoxypyrimidin-2- yl)acrylamide | 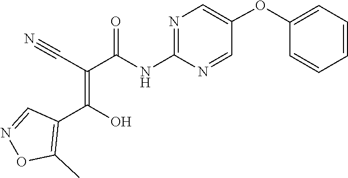 [SMILES: Cc1oncc1/C(O)=C(\C#N)C(=O)Nc1ncc(Oc2ccccc2)cn1] | 364.1 [M + H]+ ESI pos. |
| 83 | (Z)-2-cyano-3-hydroxy-3- (5-methylisoxazol-4-yl)- N-(5-phenoxypyridin-2- yl)acrylamide | 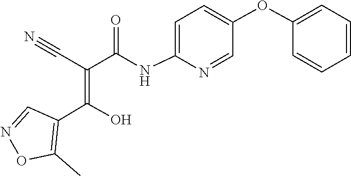 [SMILES: Cc1oncc1/C(O)=C(\C#N)C(=O)Nc1ccc(Oc2ccccc2)cn1] | 363.2 [M + H]+ ESI pos. |
| 84 | (Z)-N-(3-chloro-5- (trifluoromethyl)pyridin-2- yl)-2-cyano-3-hydroxy-3- (5-methylisoxazol-4- yl)acrylamide | 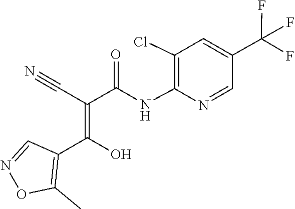 [SMILES: Cc1oncc1/C(O)=C(\C#N)C(=O)Nc1ncc(C(F)(F)F)cc1Cl] | 373.1 [M + H]+ ESI pos. |
| 85 | (Z)-2-cyano-N-(5- (cyclopentylmethoxy) pyrimidin-2-yl)-3-hydroxy- 3- (5-methylisoxazol-4- yl)acrylamide | 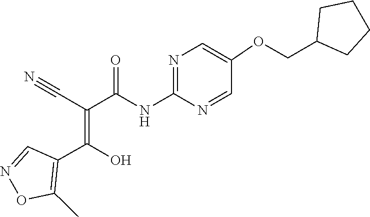 [SMILES: Cc1oncc1/C(O)=C(\C#N)C(=O)Nc1ncc(OCC2CCCC2)cn1] | 370.2 [M + H]+ ESI pos. |
| 86 | (Z)-2-cyano-3-hydroxy-3- (5-methylisoxazol-4-yl)- N-(5- (phenylethynyl)pyrimidin- 2-yl)acrylamide | 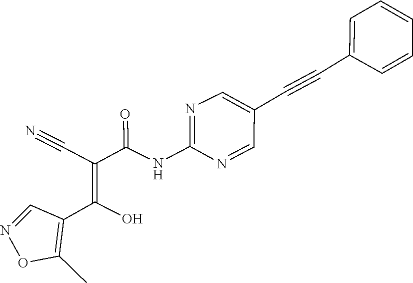 [SMILES: Cc1oncc1/C(O)=C(\C#N)C(=O)Nc1ncc(C#Cc2ccccc2)cn1] | 372.1 [M + H]+ ESI pos. |
| 87 | (Z)-2-cyano-3-hydroxy-3- (5-methylisoxazol-4-yl)- N-(4-phenethylpyrimidin- 2-yl)acrylamide | 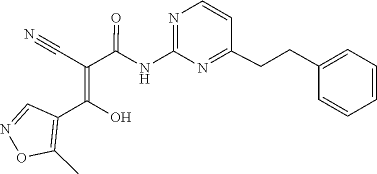 [SMILES: Cc1oncc1/C(O)=C(\C#N)C(=O)Nc1nccc(CCc2ccccc2)n1] | 376.2 [M + H]+ ESI pos. |
| 88 | (Z)-2-cyano-N-(5- (cyclohexylmethyl)pyrimidin- 2-yl)-3-hydroxy-3-(5- methylisoxazol-4- yl)acrylamide | 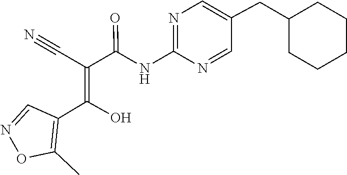 [SMILES: Cc1oncc1/C(O)=C(\C#N)C(=O)Nc1ncc(CC2CCCCC2)cn1] | 368.2 [M + H]+ ESI pos. |
| 89 | (Z)-2-cyano-3-hydroxy-N- (5-(2- methoxyethoxy)pyrimidin- 2-yl)-3-(5-methylisoxazol- 4-yl)acrylamide | 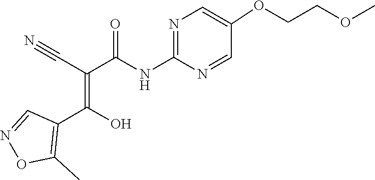 [SMILES: COCCOc1cnc(NC(=O)/C(C#N)=C(\O)c2cnoc2C)nc1] | 346.1 [M + H]+ ESI pos. |
| 90 | (Z)-2-cyano-3-hydroxy-3- (5-methylisoxazol-4-yl)- N-(5- (phenylsulfinyl)pyridin-2- yl)acrylamide | 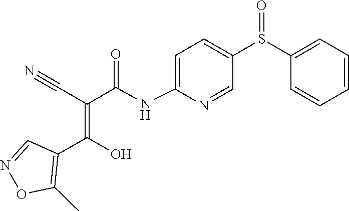 [SMILES: Cc1oncc1/C(O)=C(\C#N)C(=O)Nc1ccc(S(=O)c2ccccc2)cn1] | 395.1 [M + H]+ ESI pos. |
| 91 | (Z)-2-cyano-3-hydroxy-3- (5-methylisoxazol-4-yl)- N-(5- (methylsulfonyl)pyridin-2- yl)acrylamide | 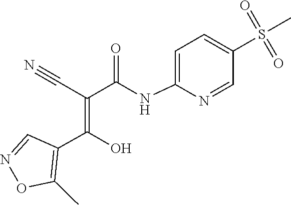 [SMILES: Cc1oncc1/C(O)=C(\C#N)C(=O)Nc1ccc(S(C)(=O)=O)cn1] | 349.1 [M + H]+ ESI pos. |
| 92 | (Z)-2-cyano-N-[3-fluoro- (5-(trifluoromethyl)-2- pyridyl]-3-hydroxy-3-(5- methylisoxazol-4-yl)prop- 2-enamide | 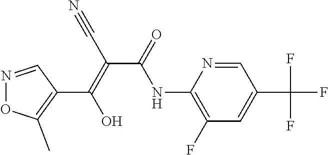 [SMILES: Cc1oncc1/C(O)=C(\C#N)C(=O)Nc1ncc(C(F)(F)F)cc1F] | 356.9 [M + H]+ |
| 93 | (Z)-N-(5-(N-(tert- butyl)sulfamoyl)pyridin-2- yl)-2-cyano-3-hydroxy-3- (5-methylisoxazol-4- yl)acrylamide | 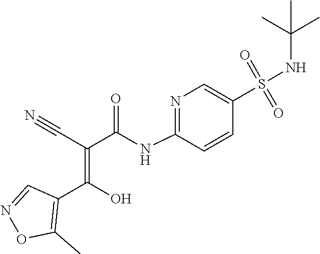 [SMILES: Cc1oncc1/C(O)=C(\C#N)C(=O)Nc1ccc(S(=O)(=O)NC(C)(C)C)cn1] | 406.2 [M + H]+ ESI pos. |
| 94 | (Z)-2-cyano-3-hydroxy-3- (5-methylisoxazol-4-yl)- N-(5- (trifluoromethyl)pyrimidin- 2-yl)acrylamide | 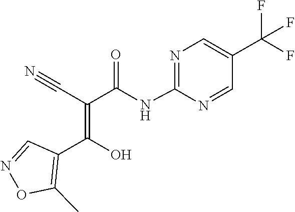 [SMILES: Cc1oncc1/C(O)=C(\C#N)C(=O)Nc1ncc(C(F)(F)F)cn1] | 340.1 [M + H]+ ESI pos. |
| 95 | (Z)-2-cyano-3-hydroxy-3- (5-methylisoxazol-4-yl)- N-(5- (phenylsulfonyl)pyridin-2- yl)acrylamide | 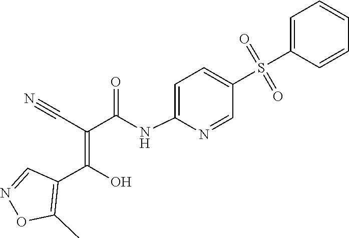 [SMILES: Cc1oncc1/C(O)=C(\C#N)C(=O)Nc1ccc(S(=O)(=O)c2ccccc2)cn1] | 411.1 [M + H]+ ESI pos. |
| 96 | (Z)-2-cyano-N-(5-(N,N- dimethylsulfamoyl) pyrimidin-2-yl)-3-hydroxy- 3-(5-methylisoxazol-4- yl)acrylamide | 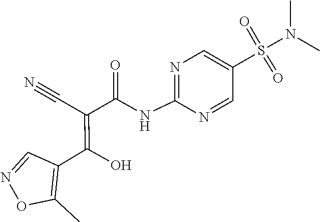 [SMILES: Cc1oncc1/C(O)=C(\C#N)C(=O)Nc1ncc(S(=O)(=O)N(C)C)cn1] | 379.2 [M + H]+ ESI pos. |
| 97 | (Z)-2-cyano-3-hydroxy-3- (5-methylisoxazol-4-yl)- N-(5- phenylsulfanylpyrimidin- 2-yl)prop-2-enamide | 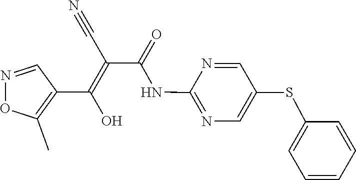 [SMILES: Cc1oncc1/C(O)=C(\C#N)C(=O)Nc1ncc(Sc2ccccc2)cn1] | 380.2 [M + H]+ |
| 98 | (Z)-2-cyano-N-(5- ethoxypyrimidin-2-yl)-3- hydroxy-3-(5- methylisoxazol-4- yl)acrylamide | 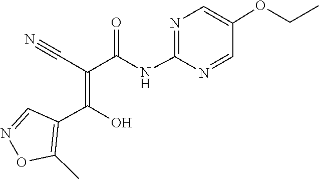 [SMILES: CCOc1cnc(NC(=O)/C(C#N)=C(\O)c2cnoc2C)nc1] | 316.1 [M + H]+ ESI pos. |
| 99 | 2-[[(Z)-2-cyano-3- hydroxy-3-(5- methylisoxazol-4-yl)prop- 2-enoyl]amino]-N-methyl- N-phenyl-pyrimidine-5- carboxamide | 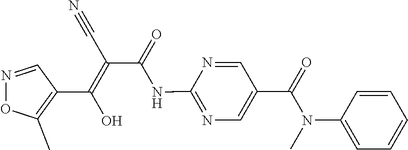 | 334.1 [M + H]+ |
| 100 | N-benzyl-2-[[(Z)-2-cyano- 3-hydroxy-3-(5- methylisoxazol-4-yl)prop- enoyl]amino]-N-methyl- 2-pyrimidine-5-carboxamide | 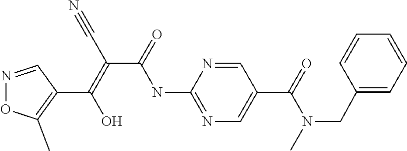 | 419.4 [M + H]+ |
| 101 | (Z)-2-cyano-N-(4-(2- fluoroethoxy)pyrimidin-2- yl)-3-hydroxy-3-(5- methylisoxazol-4- yl)acrylamide | 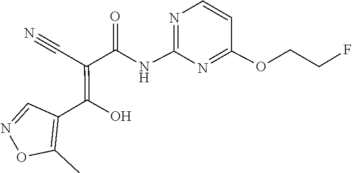 | 419.4 [M + H]+ ESI pos. |
| 102 | (Z)-2-cyano-3-hydroxy-3- (5-methylisoxazol-4-yl)- N-(5-phenethylpyrimidin- 2-yl)acrylamide | 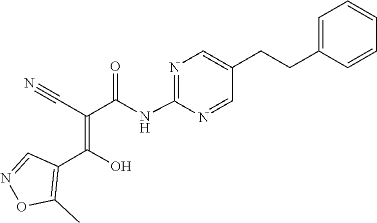 [SMILES: Cc1oncc1/C(O)=C(\C#N)C(=O)Nc1ncc(CCc2ccccc2)cn1] | 376.1 [M + H]+ ESI pos. |
| 103 | (Z)-2-cyano-N-(5- (ethylsulfonyl)pyrimidin- 2-yl)-3-hydroxy-3-(5- methylisoxazol-4- yl)acrylamide | 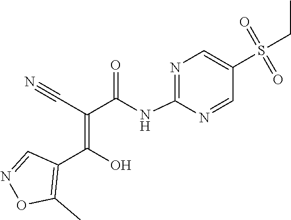 [SMILES: CCS(=O)(=O)c1cnc(NC(=O)/C(C#N)=C(\O)c2cnoc2C)nc1] | 362.1 [M − H]− ESI neg. |
| 104 | (Z)-2-cyano-3-hydroxy-3- (5-methylisoxazol-4-yl)- N-(4-(2-phenylpropan-2- yl)phenyl)acrylamide | 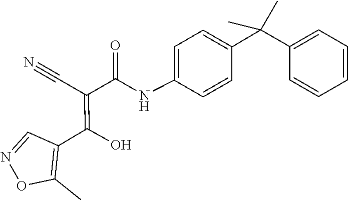 [SMILES: Cc1oncc1/C(O)=C(\C#N)C(=O)Nc1ccc(C(C)(C)c2ccccc2)cc1] | 388.2 [M + H]+ ESI pos. |
| 105 | 2-[[(Z)-2-cyano-3- hydroxy-3-(5- methylisoxazol-4-yl)prop- 2-enoyl]amino]-N-phenyl- pyrimidin-5-carboxamide | 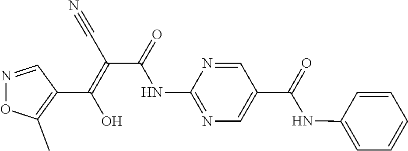 [SMILES: Cc1oncc1/C(O)=C(\C#N)C(=O)Nc1ncc(C(=O)Nc2ccccc2)cn1] | 391.0 [M + H]+ |
| 106 | N-benzyl-2-[[(Z)-2-cyano- 3-hydroxy-3-(5- methylisoxazol-4-yl)prop- 2-enoyl]amino]pyrimidine- 5-carboxamide | 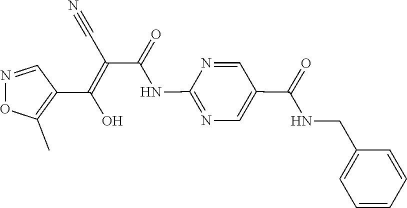 [SMILES: Cc1oncc1/C(O)=C(\C#N)C(=O)Nc1ncc(C(=O)NCc2ccccc2)cn1] | 405.2 [M + H]+ |
| 107 | (Z)-N-(5-bromopyrimidin- 2-yl)-2-cyano-3-hydroxy- 3-(5-methylisoxazol-4- yl)acrylamide | 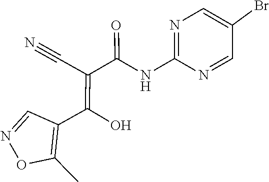 [SMILES: Cc1oncc1/C(O)=C(\C#N)C(=O)Nc1ncc(Br)cn1] | 350.0 [M + H]+ ESI pos. |
| 108 | (Z)-N-(4-chloro-6-((4- chlorophenyl)sulfonamido)- 5-phenylpyrimidin-2-yl)- 2-cyano-3-hydroxy-3-(5- methylisoxazol-4- yl)acrylamide | 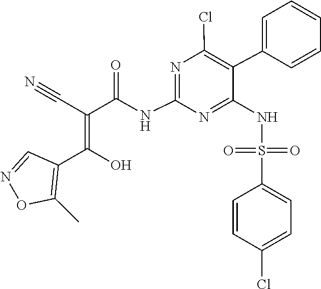 [SMILES: Cc1oncc1/C(O)=C(\C#N)C(=O)Nc1nc(Cl)c(-c2ccccc2)c(NS(=O)(=O)c2ccc(Cl)cc2)n1] | 571.2 [M + H]+ ESI pos. |

### Example 109

### (Z)—N-(5-((2-chlorobenzyl)sulfonyl)pyridin-2-yl)-2-cyano-3-hydroxy-3-(5-methylisoxazol-4-yl)acrylamide

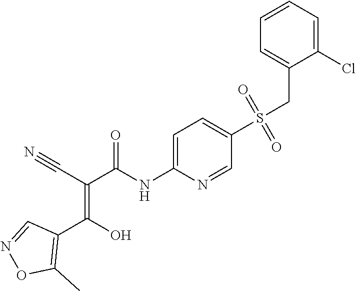 [SMILES: Cc1oncc1/C(O)=C(\C#N)C(=O)Nc1ccc(S(=O)(=O)Cc2ccccc2Cl)cn1]

### Step 1: N-(5-((2-chlorobenzyl)thio)pyridin-2-yl)-2-cyanoacetamide

To a stirred solution of 5-((2-chlorobenzyl)thio)pyridin-2-amine (125 mg, 0.5 mmol) at RT in dichloromethane (3.1 ml) under an argon atmosphere were added 2-cyanoacetic acid (63.8 mg, 750 μmol) and DCC (155 mg, 750 μmol). Stirring at RT was continued for 16 hrs. The mixture was diluted with saturated aqueous NaHCO₃ solution. The aqueous phase was back extracted with DCM. The combined organics were washed with brine, dried (MgSO₄), filtered and concentrated to leave the crude product as a light yellow liquid. The crude product was purified by silica gel chromatography using a heptane/AcOEt gradient to give the title compound (160 mg, 80% purity, 81%) as light yellow solid. MS: 318.1 [M+H]⁺ ESI pos.

### Step 2: N-(5-((2-chlorobenzyl)sulfonyl)pyridin-2-yl)-2-cyanoacetamide

A solution of N-(5-((2-chlorobenzyl)thio)pyridin-2-yl)-2-cyanoacetamide (159 mg, 0.5 mmol) in dichloromethane (2.9 ml) was cooled to 0° C. and 3-chloroperoxybenzoic acid (247 mg, 1 mmol) was added scoopwise. The reaction mixture was allowed to warm up to room temperature and stirred for 16 hrs. The reaction mixture was diluted with saturated aqueous NaHCO₃ solution and extracted with DCM. The organic phase was dried over MgSO₄, filtered, then concentrated. The crude product was purified by chromatography over silica gel using a CH₂Cl₂/MeOH gradient to give the title compound (82 mg, 80% purity, 38%) as white solid. MS: 350.1 [M+H]⁺ ESI pos.

### Step 3: (Z)—N-(5-((2-chlorobenzyl)sulfonyl)pyridin-2-yl)-2-cyano-3-hydroxy-3-(5-methylisoxazol-4-yl)acrylamide

To a stirred solution of N-(5-((2-chlorobenzyl)sulfonyl)pyridin-2-yl)-2-cyanoacetamide (82.2 mg, 235 μmol, Eq: 1) at RT in THE (1.35 ml) under an argon atmosphere was added sodium hydride 60% dispersion in mineral oil (21.6 mg, 541 μmol, Eq: 2.3). After stirring for 10 min, a solution of 5-Methyl-isoxazole-4-carbonyl chloride (37 mg, 247 μmol, Eq: 1.05) in CH₂Cl₂ (135 μl) was added in one portion. Stirring at RT was continued for 17 hrs. The mixture was carefully treated with 0.5M HCl (3 ml), diluted with brine and extracted with dichloromethane. The combined organic layers were dried (MgS₄), filtered and concentrated. The crude product was triturated with 4 ml MeOH, stirrred for 15 min. filtered, washed with MeOH and dried to give the title compound (56 mg, 500%) as off-white solid. MS: 459.1 [M+H]⁺ ESI pos.

In analogy to the procedures described in example 109, examples 110-112 were prepared starting from suitable aminopyrimidine derivative starting materials (table 2).

TABLE 2

| Ex. | Systematic name | Structure | MS |
|---|---|---|---|
| 110 | (Z)-2-cyano-3-hydroxy-N- (5-((2- methoxyethyl)sulfonyl) pyrimidin-2-yl)-3-(5- methylisoxazol-4- yl)acrylamide | 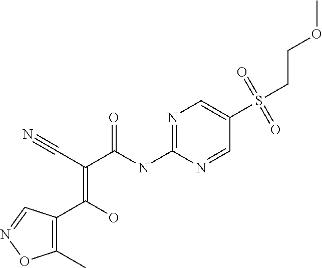 [SMILES: COCCS(=O)(=O)c1cnc(NC(=O)/C(C#N)=C(\O)c2cnoc2C)nc1] | 394.2 [M + H]+ ESI pos. |
| 111 | (Z)-N-[5- (benzenesulfonyl)pyrimidin- 2-yl]-2-cyano-3-hydroxy-3- (5-methylisoxazol-4- yl)prop-2-enamide | 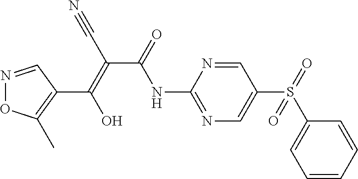 [SMILES: Cc1oncc1/C(O)=C(\C#N)C(=O)Nc1ncc(S(=O)(=O)c2ccccc2)cn1] | 412.4 [M + H]+ |
| 112 | (Z)-N-[5- (benzenesulfinyl)pyrimidin- 2-yl]-2-cyano-3-hydroxy-3- (5-methylisoxazol-4- yl)prop-2-enamide | 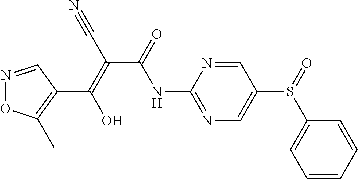 [SMILES: Cc1oncc1/C(O)=C(\C#N)C(=O)Nc1ncc(S(=O)c2ccccc2)cn1] | 396.1 [M + H]+ |

### Example 113

### (Z)-2-Cyano-3-hydroxy-3-(5-methylisoxazol-4-yl)-N-(4-(S-methylsulfonimidoyl)phenyl)acrylamide

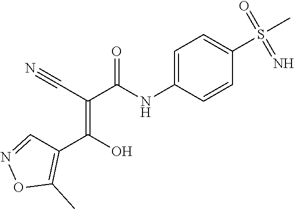 [SMILES: Cc1oncc1/C(O)=C(\C#N)C(=O)Nc1ccc(S(C)(=N)=O)cc1]

### Step 1: (Z)-2-Cyano-3-hydroxy-3-(5-methylisoxazol-4-yl)-N-(4-(methylthio)phenyl)acrylamide

In analogy to the procedures described in example 1, the title compound was prepared in two steps starting from 4-methyl-sulfanilaniline. Light brown solid. MS: 316.1 [M+H]+ ESI pos.

### Step 2: (Z)-2-Cyano-3-hydroxy-3-(5-methylisoxazol-4-yl)-N-(4-(S-methylsulfonimidoyl)phenyl)acrylamide

To a stirred mixture of (Z)-2-cyano-3-hydroxy-3-(5-methylisoxazol-4-yl)-N-(4-(methylthio)phenyl)acrylamide (27 mg, 85.6 μmol) at RT in ethanol (856 μl) under an argon atmosphere was added (diacetoxyiodo)benzene (82.7 mg, 257 μmol) and ammonium acetate (26.4 mg, 342 μmol). Stirring at RT was continued for 1 hr. The solution was evaporated to dryness, then extracted with H₂O and dichloromethane. The inorganic phase was acidified with 1N HCl (1 ml) and extracted with DCM. The combined organics were dried (MgSO₄), filtered and concentrated to leave the title compound (12 mg, 35%) as an off-white solid. MS: 347.2 [M+H]+ ESI pos.

In analogy to the procedures described in example 109, examples 114-116 were prepared using a suitable oxidant in the last step (table 3).

TABLE 3

| Ex. | Oxidant | Systematic name | Structure | MS |
|---|---|---|---|---|
| 114 | (diacetoxy- iodo) benzene | (Z)-2-cyano-3-hydroxy- 3-(5-methylisoxazol-4- yl)-N-(4- (phenylsulfonimidoyl) phenyl)acrylamide | 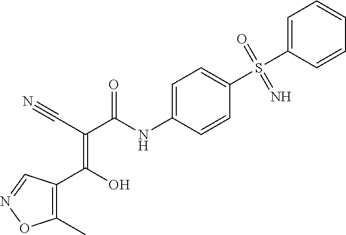 [SMILES: Cc1oncc1/C(O)=C(\C#N)C(=O)Nc1ccc(S(=N)(=O)c2ccccc2)cc1] | 409.2 [M + H]+ ESI pos. |
| 115 | 3- chloro- peroxy- benzoic acid | (Z)-2-cyano-3-hydroxy- 3-(5-methylisoxazol-4- yl)-N-(4- methylsulfonylphenyl) prop-2-enamide | 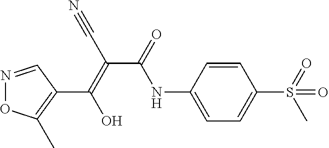 [SMILES: Cc1oncc1/C(O)=C(\C#N)C(=O)Nc1ccc(S(C)(=O)=O)cc1] | 348.3 [M + H]+ |
| 116 | 3- chloro- peroxy- benzoic acid | (Z)-2-cyano-3-hydroxy- 3-(5-methylisoxazol-4- yl)-N-(4- methylsulfinylphenyl) prop-2-enamide | 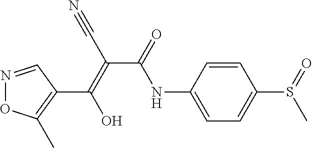 [SMILES: Cc1oncc1/C(O)=C(\C#N)C(=O)Nc1ccc(S(C)=O)cc1] | 332.3 [M + H]+ |

### Example 117

### (Z)-2-Cyano-3-hydroxy-N-(5-(N-(2-methoxyethyl)-N-methylsulfamoyl)pyrimidin-2-yl)-3-(5-methylisoxazol-4-yl)acrylamide

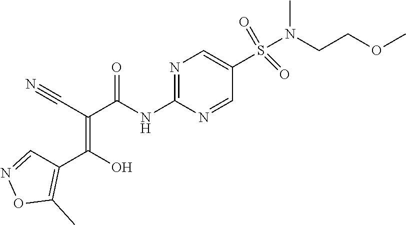 [SMILES: COCCN(C)S(=O)(=O)c1cnc(NC(=O)/C(C#N)=C(\O)c2cnoc2C)nc1]

### Step 1: 2-Amino-N-(2-methoxyethyl)-N-methylpyrimidine-5-sulfonamide

A mixture of 2-aminopyrimidine-5-sulfonyl chloride (252 mg, 1.3 mmol) in dichloromethane (720 μl) was cooled to 0° C., Et₃N (395 mg, 544 μl, 3.9 mmol) and 2-methoxy-N-methylethan-1-amine (127 mg, 1.4 mmol) was added. The reaction mixture was allowed to warm up to room temperature and stirred for 2 hrs. The reaction mixture was diluted with water and extracted with DCM. The organic phase was dried over MgSO₄, filtered, then concentrated to afford the title compound (303 mg, 90%) as an off-white solid. MS: 247.2 [M+H]+ ESI pos.

### Step 2: (Z)-2-Cyano-3-hydroxy-N-(5-(N-(2-methoxyethyl)-N-methylsulfamoyl)pyrimidin-2-yl)-3-(5-methylisoxazol-4-yl)acrylamide

In analogy to the procedures described in example 1, 2-amino-N-(2-methoxyethyl)-N-methylpyrimidine-5-sulfonamide was converted into the title compound in two steps. Yellow solid. MS: 432.2 [M+H]+ ESI pos.

In analogy to the procedures described in example 117, example 118 was prepared using 2-methoxyethan-1-amine in the first step (table 4).

TABLE 4

| Ex. | Systematic name | Structure | MS |
|---|---|---|---|
| 118 | (Z)-2-cyano-3-hydroxy-N- (5-(N-(2-methoxy- ethyl)sulfamoyl)pyrimidin- 2-yl)-3-(5-methylisoxazol- 4-yl)acrylamide | 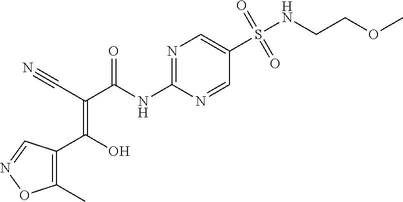 [SMILES: COCCNS(=O)(=O)c1cnc(NC(=O)/C(C#N)=C(\O)c2cnoc2C)nc1] | 407.3 [M − H]− ESI neg. |

### Example 119

### (Z)-2-Cyano-3-hydroxy-N-(4-(N-(2-methoxyethyl)-N-methylsulfamoyl)phenyl)-3-(5-methylisoxazol-4-yl)acrylamide

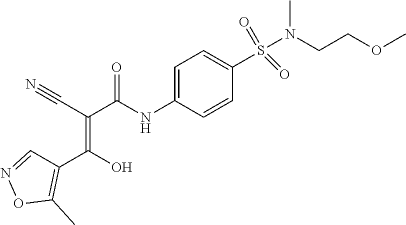 [SMILES: COCCN(C)S(=O)(=O)c1ccc(NC(=O)/C(C#N)=C(\O)c2cnoc2C)cc1]

### Step 1: N-(2-Methoxyethyl)-N-methyl-4-nitrobenzenesulfonamide

A mixture of 4-nitrobenzenesulfonyl chloride (443 mg, 2 mmol) in dichloromethane (1.1 ml) was cooled to 0° C., Et₃N (607 mg, 836 μl, 6 mmol) and 2-methoxy-N-methylethan-1-amine (196 mg, 2.2 mmol) were added. The reaction mixture was allowed to warm up to room temperature and stirred for 16 hrs. The reaction mixture was diluted with water and extracted with dichloromethane. The organic phase was dried over MgSO₄, filtered, then concentrated. The crude product was purified by chromatography on silica gel using a heptane/AcOEt gradient as eluent to obtain the title compound (405 mg, 70%) as light yellow solid. MS: 275.1 [M+H]+ ESI pos.

### Step 2: 4-Amino-N-(2-methoxyethyl)-N-methylbenzenesulfonamide

To a mixture of N-(2-methoxyethyl)-N-methyl-4-nitrobenzenesulfonamide (0.402 g, 1.47 mmol) in methanol (9.85 ml) was added palladium on carbon 10% (40 mg, 378 μmol) and the mixture was vigorously stirred under H₂ atmosphere over night. The catalyst was filtered off and washed with methanol. The filtrate was concentrated. The crude product was purified by silica gel chromatography using a heptane/EtOAc gradient as eluent to provide the title compound as light yellow solid. MS: 245.1 [M+H]+ ESI pos.

### Step 3: (Z)-2-Cyano-3-hydroxy-N-(4-(N-(2-methoxyethyl)-N-methylsulfamoyl)phenyl)-3-(5-methylisoxazol-4-yl)acrylamide

In analogy to the procedures described in example 1, 4-amino-N-(2-methoxyethyl)-N-methylbenzenesulfonamide was converted into the title compound in two steps. Light yellow solid. MS: 421.3 [M+H]+ ESI pos.

In analogy to the procedures described in example 119, example 120 was prepared using N1,N1,N2-trimethylethane-1,2-diamine in the first step (table 5).

TABLE 5

| Ex. | Systematic name | Structure | MS |
|---|---|---|---|
| 120 | (Z)-2-cyano-N-(4-(N-(2- (dimethylamino)ethyl)-N- methylsulfamoyl)phenyl)- 3-hydroxy-3-(5- methylisoxazol-4- yl)acrylamide | 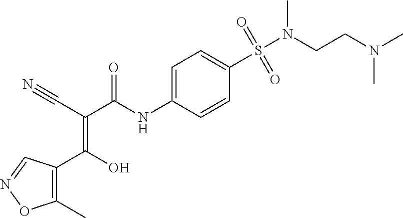 [SMILES: Cc1oncc1/C(O)=C(\C#N)C(=O)Nc1ccc(S(=O)(=O)N(C)CCN(C)C)cc1] | 434.3 [M + H]+ |

### Example 121

### (Z)-2-Cyano-N-(4-((4-cyanophenyl)sulfonyl)phenyl)-3-hydroxy-3-(5-methylisoxazol-4-yl)acrylamide

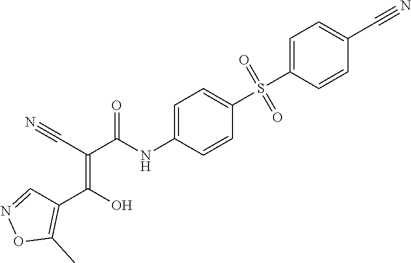 [SMILES: Cc1oncc1/C(O)=C(\C#N)C(=O)Nc1ccc(S(=O)(=O)c2ccc(C#N)cc2)cc1]

### Step 1: 4-((4-Aminophenyl)sulfonyl)benzonitrile

A solution of 4-((4-aminophenyl)thio)benzonitrile (339 mg, 1.5 mmol) in dichloromethane (9 ml) was cooled to 0° C. and 3-chloroperoxybenzoic acid (840 mg, 3.75 mmol) was added scoopwise. The reaction mixture was allowed to warm up to room temperature and stirred for 2 hrs. The reaction mixture was diluted with aqueous sodium carbonate (pH=basic) and extracted with dichloromethane. The organic phase was dried over MgSO₄, filtered, then concentrated. The crude product (0.38 g) was purified by silica gel chromatography using a heptane/AcOEt gradient as eluent to provide the title compound (380 mg, 95%) as yellow solid. MS: 259.1 [M+H]+ ESI pos.

### Steps 2 & 3: (Z)-2-Cyano-N-(4-((4-cyanophenyl)sulfonyl)phenyl)-3-hydroxy-3-(5-methylisoxazol-4-yl)acrylamide

In analogy to the procedures described in example 1, 4-((4-aminophenyl)sulfonyl)benzonitrile was converted into the title compound in two steps as a light yellow solid. MS: 433.1 [M−H]− ESI neg.

In analogy to the procedures described in example 121, examples 122 and 123 were prepared starting from a suitable thioether (table 6).

TABLE 6

| Ex. | Systematic name | Structure | MS |
|---|---|---|---|
| 122 | (Z)-2-cyano-N-(4-((2- cyanophenyl)sulfonyl) phenyl)-3-hydroxy-3-(5- methylisoxazol-4- yl)acrylamide | 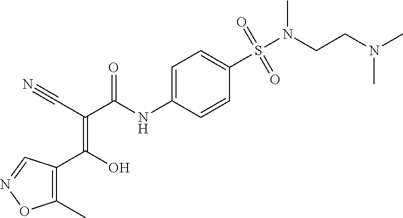 [SMILES: Cc1oncc1/C(O)=C(\C#N)C(=O)Nc1ccc(S(=O)(=O)N(C)CCN(C)C)cc1] | 433.2 [M − H]− |
| 123 | (Z)-2-cyano-3-hydroxy-3- (5-methylisoxazol-4-yl)-N- (4-(pyridin-3- ylsulfonyl)phenyl) acrylamide | 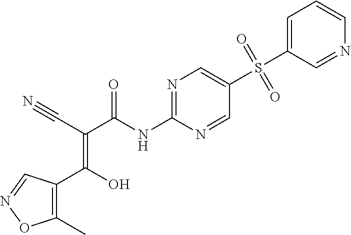 [SMILES: Cc1oncc1/C(O)=C(\C#N)C(=O)Nc1ncc(S(=O)(=O)c2cccnc2)cn1] | 411.2 [M + H]+ |

### Example 124

### ((Z)-2-cyano-3-hydroxy-N-(5-methoxy-4-phenylpyrimidin-2-yl)-3-(5-methylisoxazol-4-yl)acrylamide

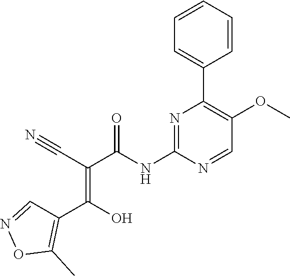 [SMILES: COc1cnc(NC(=O)/C(C#N)=C(\O)c2cnoc2C)nc1-c1ccccc1]

### Step 1: 5-(2-methoxy)-4-phenylpyrimidin-2-amine

In a vial suitable for microwave chemistry, a mixture of 2-chloro-5-(2-methoxyethoxy)-4-phenylpyrimidine (90 mg, 0.34 mmol), diphenylmethanimine (80.1 mg, 74.2 μl, 442 μmol), BINAP (21.2 mg, 34 μmol), palladium (II) acetate (7.63 mg, 34 μmol) and cesium carbonate (277 mg, 850 μmol) in 1,4-dioxane (1.56 ml) was stirred at 120° C. over night. The mixture was cooled to RT and diluted with H₂O and EtOAc. The aqueous phase was back extracted with EtOAc. The combined organics were washed with brine, dried (MgSO₄), filtered and concentrated.

The crude imine intermediate (0.0833 g) was dissolved in methanol (4.7 ml) and hydroxylamine hydrochloride (56.7 mg, 816 μmol) and sodium acetate (167 mg, 2.04 mmol) were added. The reaction mixture was stirred at 23° C. over night, then directly purified by silica gel chromatography using a dichloromethane/methanol gradient as eluent to provide the title compound (42 mg, 48%) as off white solid. MS: 246.2 [M+H]⁺ ESI pos.

### Step 2: (Z)-2-cyano-3-hydroxy-N-(5-methoxy-4-phenylpyrimidin-2-yl)-3-(5-methylisoxazol-4-yl)acrylamide

In analogy to the procedures described in example 1, 5-(2-methoxy)-4-phenylpyrimidin-2-amine was converted into the title compound in two steps as a light yellow solid. MS: 378.2 [M+H]⁺ ESI pos.

In analogy to the procedures described in example 124, examples 125-127 were prepared starting from a suitable chloropyrimidine (table 7).

TABLE 7

| Ex. | Systematic name | Structure | MS |
|---|---|---|---|
| 125 | (Z)-N-(4-(4- chlorophenyl)-5- (cyclopropylmethoxy) pyrimidin-2-yl)-2- cyano-3-hydroxy-3-(5- methylisoxazol-4- yl)acrylamide | 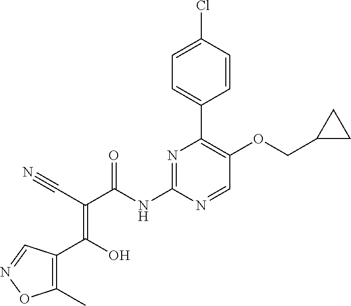 [SMILES: Cc1oncc1/C(O)=C(\C#N)C(=O)Nc1ncc(OCC2CC2)c(-c2ccc(Cl)cc2)n1] | 452.2 [M + H]+ ESI pos. |
| 126 | (Z)-2-cyano-3-hydroxy- N-(5-(2-methoxyethoxy)- 4-phenylpyrimidin-2-yl)- 3-(5-methylisoxazol-4- yl)acrylamide | 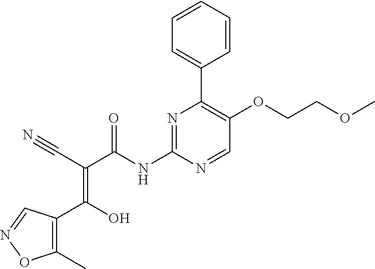 [SMILES: COCCOc1cnc(NC(=O)/C(C#N)=C(\O)c2cnoc2C)nc1-c1ccccc1] | 422.2 [M + H]+ ESI pos. |
| 127 | (Z)-N-(4-(4- chlorophenyl)-5-(2,2,2- trifluoroethoxy)pyrimidin- 2-yl)-2-cyano-3- hydroxy-3-(5- methylisoxazol-4- yl)acrylamide | 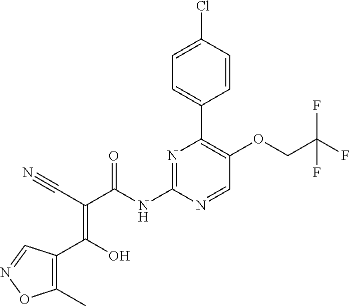 [SMILES: Cc1oncc1/C(O)=C(\C#N)C(=O)Nc1ncc(OCC(F)(F)F)c(-c2ccc(Cl)cc2)n1] | 480.2 [M + H]+ ESI pos. |

### Example 128

### (Z)-2-cyano-3-hydroxy-N-(5-(2-methoxyethoxy)-4-phenethylpyrimidin-2-yl)-3-(5-methylisoxazol-4-yl)acrylamide

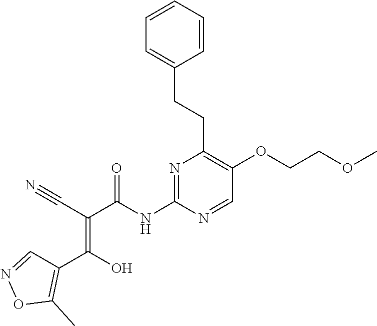 [SMILES: COCCOc1cnc(NC(=O)/C(C#N)=C(\O)c2cnoc2C)nc1CCc1ccccc1]

### Step 1: 2-Chloro-5-fluoro-4-(phenylethynyl)pyrimidine

In a vial suitable for microwave chemistry, 2,4-dichloro-5-fluoropyrimidine (111 mg, 662 μmol) was dissolved in DMF (2.96 ml) and ethynylbenzene (135 mg, 145 μl, 1), triethylamine (134 mg, 184 μl, 1.32 mmol), tetrakis(triphenylphosphine)-palladium(O) (22.9 mg, 19.9 μmol)) and copper(I) iodide (1.26 mg, 6.62 μmol) were added at RT under nitrogen and the vial was closed. The mixture was stirred for 2 hr at 80° C. (oil bath temperature was 85° C.). Then, the mixture was cooled to 23° C., diluted with H₂O and EtOAc. The aqueous phase was back extracted with EtOAc. The combined organics were washed with brine, dried (MgSO₄), filtered and concentrated. The crude product was purified by silica gel chromatography using a heptane/EtOAc gradient as eluent to provide the title compound (130 mg, 82%) as yellow solid. MS: 233.1 [M+H]+ ESI pos.

### Step 2: 2-Chloro-5-(2-methoxyethoxy)-4-(phenylethynyl)pyrimidine

A solution of 2-methoxyethan-1-ol (46.7 mg, 48.4 μL, 614 μmol) in DMF (691 μl) was cooled to 0° C. and sodium hydride 60% dispersion in mineral oil (24.5 mg, 614 μmol) was added portionwise. The reaction mixture was stirred at 23° C. for 15 min. The resulting suspension was added at 0° C. dropwise to a solution of 2-chloro-5-fluoro-4-(phenylethynyl)pyrimidine (130 mg, 558 μmol) in DMF (691 μl) at 0° C. to give a yellow solution. The reaction mixture was stirred at 23° C. for 2 h. The reaction mixture was quenched at 0° C. with water and extracted with EtOAc. The combined organics were washed with brine, dried (MgSO₄), filtered and concentrated. The crude product was purified by silica gel chromatography using a heptane/EtOAc gradient as eluent to provide the title compound (106 mg, 63%) as orange solid. MS: 289.1 [M+H]+ ESI pos.

### Step 3: 5-(2-Methoxyethoxy)-4-(phenylethynyl)pyrimidin-2-amine

In a vial suitable for microwave chemistry, a mixture of 2-chloro-5-(2-methoxyethoxy)-4-(phenylethynyl)pyrimidine (106 mg, 0.367 mmol), diphenylmethanimine (86.5 mg, 80.1 μl, 477 μmol), BINAP (22.9 mg, 36.7 μmol), palladium (II) acetate (8.24 mg, 36.7 μmol) and cesium carbonate (299 mg, 917 μmol) in 1,4-dioxane (1.68 ml) was stirred at 120° C. (oil bath temperature) over night. The mixture was cooled to 23° C., diluted with H₂O and EtOAct. The aqueous phase was back extracted with EtOAc. The combined organics were washed with brine, dried (MgSO₄), filtered and concentrated. The crude product was purified by silica gel chromatography using a heptane/EtOAc gradient as eluent.

The imine intermediate (0.115 g) was dissolved in methanol (5 ml) and hydroxylamine hydrochloride (61.2 mg, 881 μmol) and sodium acetate (181 mg, 2.2 mmol) were added. The reaction mixture was stirred at 23° C. overnight. The reaction mixture was directly purified by silica gel chromatography using a dicholoromethane/MeOH gradient as eluent to provide the title compound (46 mg, 44%) as yellow liquid. MS: 270.2 [M+H]+ ESI pos.

### Step 4: 5-(2-Methoxyethoxy)-4-phenethylpyrimidin-2-amine

To the solution of 5-(2-methoxyethoxy)-4-(phenylethynyl)pyrimidin-2-amine (45.8 mg, 0.17 mmol) at RT in ethanol (833 μl) was added 10% Pd/C (3.62 mg, 3.4 μmol). The reaction mixture was degassed and back-filled with H₂. The black suspension was stirred at RT under a hydrogen atmosphere for 18 hrs. The catalyst was filtered off and washed with EtOH. The filtrate was concentrated to leave the title compound (41 mg, 86%) as a light yellow solid. MS: 274.2 [M+H]⁺ ESI pos.

### Steps 5 & 6: (Z)-2-Cyano-3-hydroxy-N-(5-(2-methoxyethoxy)-4-phenethylpyrimidin-2-yl)-3-(5-methylisoxazol-4-yl)acrylamide

In analogy to the procedures described in example 1, 5-(2-methoxyethoxy)-4-phenethylpyrimidin-2-amine was converted into the title compound in two steps. Light yellow solid. MS: 450.3 [M+H]⁺ ESI pos.

In analogy to the procedures described in example 128, example 129 were prepared using methanolate in the second step (table 8).

TABLE 8

| Ex. | Systematic name | Structure | MS |
|---|---|---|---|
| 129 | (Z)-2-cyano-3-hydroxy- N-(5-methoxy-4- phenethylpyrimidin-2- yl)-3-(5-methylisoxazol- 4-yl)acrylamide | 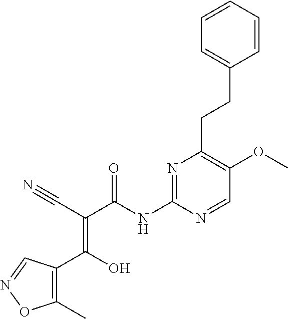 [SMILES: COc1cnc(NC(=O)/C(C#N)=C(\O)c2cnoc2C)nc1CCc1ccccc1] | 406.2 [M + H]+ ESI pos. |

### Example 130

### (Z)-2-cyano-3-hydroxy-N-(5-(2-(6-methoxypyridin-3-yl)ethyl)-4-(trifluoromethyl)pyrimidin-2-yl)-3-(5-methylisoxazol-4-yl)acrylamide

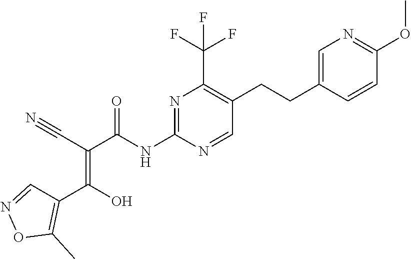 [SMILES: COc1ccc(CCc2cnc(NC(=O)/C(C#N)=C(\O)c3cnoc3C)nc2C(F)(F)F)cn1]

### Step 1: 5-((6-Methoxypyridin-3-yl)ethynyl)-4-(trifluoromethyl)pyrimidin-2-amine

In a vial suitable for microwave chemistry, 5-bromo-4-(trifluoromethyl)pyrimidin-2-amine (242 mg, 1 mmol) was dissolved in DMF (4.47 ml) and 5-ethynyl-2-methoxypyridine (266 mg, 2 mmol, Eq: 2), triethylamine (202 mg, 278 μl, 2 mmol), tetrakis(triphenylphosphine)-palladium(0) (34.7 mg, 30 μmol) and copper(I) iodide (1.9 mg, 10 μmol) were added at RT under nitrogen and the vial was closed. The mixture was stirred for 16 hrs at 80° C. (oil bath temperature was 85° C.). The mixture was cooled to 23° C., diluted with H₂O and EtOAc. The aqueous phase was back extracted with EtOAc. The combined organics were washed with brine, dried (MgSO₄), filtered and concentrated. The crude product was purified by silica gel chromatophraphy using a heptane/EtOAc gradient as eluent to provide the title compound (167 mg, 57%) as light yellow solid. MS: 295.1 [M+H]+ ESI pos.

### Step 2: 5-(2-(6-Methoxypyridin-3-yl)ethyl)-4-(trifluoromethyl)pyrimidin-2-amine

To a mixture of 5-((6-methoxypyridin-3-yl)ethynyl)-4-(trifluoromethyl)pyrimidin-2-amine at RT in MeOH (5.5 ml) was added 10% Pd/C (11.9 mg, 11.2 μmol). The reaction mixture was degassed and back-filled with H₂. The black suspension was stirred at RT under a hydrogen atmosphere for 18 hrs. The catalyst was filtered off and washed with MeOH. The filtrate was concentrated to leave the title compound (162 mg, 97%) as a white solid. MS: 299.2 [M+H]+ ESI pos.

### Steps 3 & 4: (Z)-2-cyano-3-hydroxy-N-(5-(2-(6-methoxypyridin-3-yl)ethyl)-4-(trifluoromethyl)pyrimidin-2-yl)-3-(5-methylisoxazol-4-yl)acrylamide

In analogy to the procedures described in example 1, 5-(2-(6-methoxypyridin-3-yl)ethyl)-4-(trifluoromethyl)pyrimidin-2-amine was converted in two steps into the title compound as a yellow solid. MS: 475.3 [M+H]+ ESI pos.

In analogy to the procedures described in example 130, example 131 was prepared using ethinyl benzene in the second step (table 9).

TABLE 9

| Ex. | Systematic name | Structure | MS |
|---|---|---|---|
| 131 | (Z)-2-cyano-3-hydroxy-3- (5-methylisoxazol-4-yl)-N- (5-phenethyl-4- (trifluoromethyl)pyrimidin- 2-yl)acrylamide | 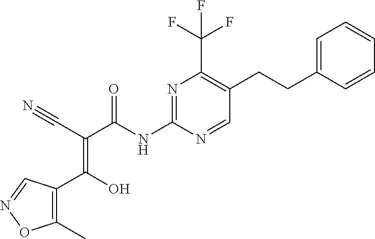 [SMILES: Cc1oncc1/C(O)=C(\C#N)C(=O)Nc1ncc(CCc2ccccc2)c(C(F)(F)F)n1] | 444.3 [M + H]+ ESI pos. |

### Example 132

### ((Z)—N-Benzyl-2-(2-cyano-3-hydroxy-3-(5-methylisoxazol-4-yl)acrylamido)-N-methyl-4-phenylpyrimidine-5-carboxamide

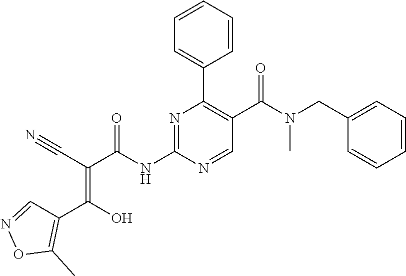 [SMILES: Cc1oncc1/C(O)=C(\C#N)C(=O)Nc1ncc(C(=O)N(C)Cc2ccccc2)c(-c2ccccc2)n1]

### Step 1: Methyl (Z)-2-benzoyl-3-(dimethylamino)acrylate

To a stirred solution of methyl 3-oxo-3-phenylpropanoate (356 mg, 2 mmol) at RT in toluene (3.25 ml) under an argon atmosphere was added 1,1-dimethoxy-N,N-dimethylmethanamine (572 mg, 638 μl, 4.8 mmol) in one portion. The mixture was heated to 110° C. (oil bath temperature) and stirring was continued for 2 hrs. The clear yellow solution was cooled to 23° C. and directly purified by silica gel chromatography using a heptane/EtOAc gradient as eluent to provide the title compound (486 mg, 96%) as light yellow solid. MS: 234.2 [M+H]⁺ ESI pos.

### Step 2: Methyl 2-amino-4-phenylpyrimidine-5-carboxylate

To a stirred suspension of methyl (Z)-2-benzoyl-3-(dimethylamino)acrylate (467 mg, 2 mmol) at RT in methanol (9.8 ml) and H₂O (982 μl) under an argon atmosphere were added guanidine hydrochloride (258 mg, 2.7 mmol) and sodium carbonate (148 mg, 1.4 mmol). The mixture was heated to 75° C. (oil bath temperature) and stirring was continued for 2 h. The mixture was cooled to RT. The clear light yellow solution was diluted with H₂O. A white precipitate formed. The solid was collected by filtration, washed with plenty of water, and dried to provide the title compound (200 mg, 41%) as white solid. 230.2 [M+H]⁺ ESI pos.

### Step 3: 2-Amino-4-phenylpyrimidine-5-carboxylic acid

To a solution of methyl 2-amino-4-phenylpyrimidine-5-carboxylate (200 mg, 0.872 mmol) in methanol (772 μl), tetrahydrofuran (772 μl) and water (386 μl), lithium hydroxide monohydrate (110 mg, 2.62 mmol) was added and the reaction mixture stirred at RT over night. After additionally addition of lithium hydroxide monohydrate (110 mg, 2.62 mmol) and methanol (772 μl), tetrahydrofuran (772 μl) and water (386 μl), the reaction mixture was stirred for further 24 hrs.

The reaction mixture was concentrated to dryness. The solid was acidified with 5 ml aqueous 1N HCl solution. The suspension was stirred at around 23° C. for 30 minutes, filtered, washed with H₂O and dried at high vacuum to provide the title compound (155 mg, 78%) as white solid. MS: 216.2 [M+H]+ ESI pos.

### Step 4: 2-Amino-N-benzyl-N-methyl-4-phenylpyrimidine-5-carboxamide

To a stirred mixture of 2-amino-4-phenylpyrimidine-5-carboxylic acid (92.5 mg, 0.43 mmol) in DMF (890 μl) was added EDC (165 mg, 860 μmol), Et₃N (174 mg, 240 μl, 1.72 mmol) and N-methyl-1-phenylmethanamine (52.1 mg, 54.9 μl, 430 μmol). The reaction mixture was stirred at RT over the weekend. The solution was diluted with H₂O and washed with EtOAc. The aqueous phase was back extracted with EtOAc. The combined organics were washed with brine, dried (MgSO₄), filtered and concentrated. The crude product was purified over silica gel chromatography using a dichloromethane/MeOH gradient as eluent to provide the title compound (69 mg, 50%) as light yellow solid. MS: 319.3 [M+H]+ ESI pos.

### Steps 5 & 6: (Z)—N-Benzyl-2-(2-cyano-3-hydroxy-3-(5-methylisoxazol-4-yl)acrylamido)-N-methyl-4-phenylpyrimidine-5-carboxamide

In analogy to the procedures described in example 1, 2-amino-N-benzyl-N-methyl-4-phenylpyrimidine-5-carboxamide was converted into the title compound in two steps. Off-white solid. MS: 495.2 [M+H]+ ESI pos.

In analogy to the procedures described in example 132, examples 133-135 were prepared using a suitable β-ketoester in the first step and a suitable amine in the fourth step (table 10).

TABLE 10

| Ex. | Systematic name | Structure | MS |
|---|---|---|---|
| 133 | (Z)-N-benzyl-2-(2-cyano- 3-hydroxy-3-(5- methylisoxazol-4- yl)acrylamido)-N-methyl- 4-phenethylpyrimidine-5- carboxamide | 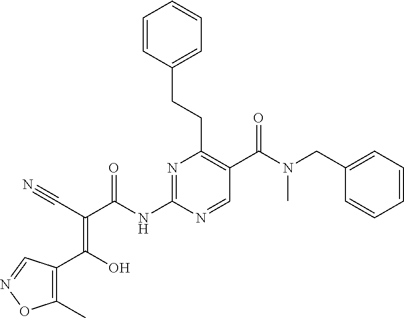 [SMILES: Cc1oncc1/C(O)=C(\C#N)C(=O)Nc1ncc(C(=O)N(C)Cc2ccccc2)c(CCc2ccccc2)n1] | 523.2 [M + H]+ |
| 134 | (Z)-2-(2-cyano-3-hydroxy- 3-(5-methylisoxazol-4- yl)acrylamido)-N-methyl- 4-phenethyl-N- phenylpyrimidine-5- carboxamide | 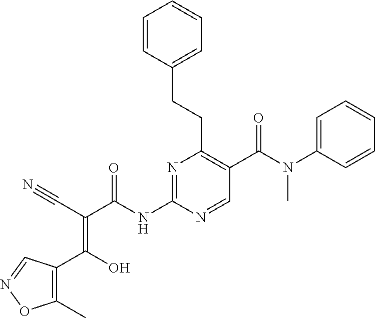 [SMILES: Cc1oncc1/C(O)=C(\C#N)C(=O)Nc1ncc(C(=O)N(C)c2ccccc2)c(CCc2ccccc2)n1] | 509.2 [M + H]+ |
| 135 | (Z)-2-(2-cyano-3-hydroxy- 3-(5-methylisoxazol-4- yl)acrylamido)-N-methyl- N,4-diphenylpyrimidine-5- carboxamide | 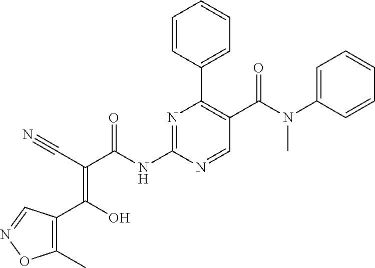 [SMILES: Cc1oncc1/C(O)=C(\C#N)C(=O)Nc1ncc(C(=O)N(C)c2ccccc2)c(-c2ccccc2)n1] | 481.3 [M + H]+ ESI pos. |

### Example 136

### (Z)—N-(4-(3-chlorophenyl)-5-(methylsulfonyl)pyrimidin-2-yl)-2-cyano-3-hydroxy-3-(5-methylisoxazol-4-yl)acrylamide

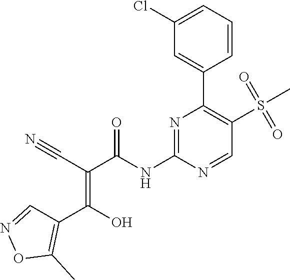 [SMILES: Cc1oncc1/C(O)=C(\C#N)C(=O)Nc1ncc(S(C)(=O)=O)c(-c2cccc(Cl)c2)n1]

### Step 1: 1-(3-chlorophenyl)-2-(methylsulfonyl)ethan-1-one

To a stirred solution of (methylsulfonyl)methane (1.53 g, 16.2 mmol at RT in DMSO extra dry (6 ml) under an argon atmosphere (important, avoid moisture from air!) was added sodium hydride 60% dispersion in mineral oil (433 mg, 10.8 mmol). Gentle bubbling. The mixture was heated to 55° C. (oil bath temperature) and stirring was continued for 2 h. The oil bath was removed. The mixture was cooled to RT and ethyl 3-chlorobenzoate (1 g, 843 μl, 5.42 mmol) was added dropwise for 5 minutes (caution: exothermic, strong bubbling). Stirring at RT was then continued for 90 minutes when a thick brown mixture, with a lot of foam formed. The reaction mixture was carefully treated with H₂O (30 ml), bubbling/exothermic at the beginning. The mixture was then treated with AcOH until pH ˜ 6 was reached and precipitation occurred. The solid was collected by filtration, then washed with H₂O and dried. The crude product was purified by silica gel chromatography using a n-heptane/EtOAc gradient as eluent to obtain the title compound (1.01 g, 76%) as off white solid. 233.0 [M+H]⁺ ESI pos.

### Step 2: (Z)-1-(3-Chlorophenyl)-3-(dimethylamino)-2-(methylsulfonyl)prop-2-en-1-one

To a stirred solution of 1-(3-chlorophenyl)-2-(methylsulfonyl)ethan-1-one (1.005 g, 4.32 mmol) at RT in toluene (8 ml) under an argon atmosphere was added 1,1-dimethoxy-N,N-dimethylmethanamine (1.24 g, 1.38 ml, 10.4 mmol) in one portion. The mixture was heated to 110° C. (oil bath temperature) and stirring was continued for 2 hrs. The clear brown solution was cooled to RT and concentrated. The crude product was purified by silica gel chromatography using a n-heptane/EtOAc gradient as eluent to provide the title compound (1.04 g, 76%) as yellow gum. 288.1 [M+H]⁺ ESI pos.

### Step 3: 4-(3-Chlorophenyl)-5-(methylsulfonyl)pyrimidin-2-amine

To a stirred suspension of (Z)-1-(3-chlorophenyl)-3-(dimethylamino)-2-(methylsulfonyl)prop-2-en-1-one (710 mg, 2.47 mmol) at RT in methanol (10 ml) and H₂O (1 ml) under an argon atmosphere were added guanidine hydrochloride (318 mg, 3.33 mmol) and sodium carbonate (183 mg, 1.73 mmol). The mixture was heated to 70° C. (oil bath temperature) and stirring was continued for 2 hrs. The mixture was cooled to RT. A solid precipitated out upon cooling. The thick suspension was diluted with H₂O. The solid was collected by filtration, washed well with water and dried to give the title compound (488 mg, 66%) as off-white powder. 284.2 [M+H]⁺ ESI pos.

### Steps 4 & 5: (Z)—N-(4-(3-Chlorophenyl)-5-(methylsulfonyl)pyrimidin-2-yl)-2-cyano-3-hydroxy-3-(5-methylisoxazol-4-yl)acrylamide

In analogy to the procedures described in example 1, 4-(3-chlorophenyl)-5-(methylsulfonyl)-pyrimidin-2-amine was converted in two steps into the title compound as a light yellow solid. MS: 460.2 [M+H]+ ESI pos.

In analogy to the procedures described in example 136, examples 137-140 were prepared using a suitable ester and a suitable methylsulfone in the first step (table 11).

TABLE 11

| Ex. | Systematic name | Structure | MS |
|---|---|---|---|
| 137 | (Z)-2-cyano-N-(4-(3- fluorophenyl)-5- (methylsulfonyl)pyrimidin- 2-yl)-3-hydroxy-3-(5- methylisoxazol-4- yl)acrylamide | 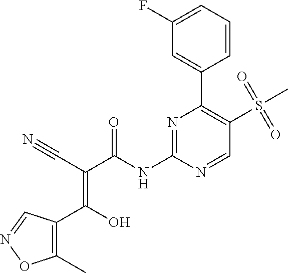 [SMILES: Cc1oncc1/C(O)=C(\C#N)C(=O)Nc1ncc(S(C)(=O)=O)c(-c2cccc(F)c2)n1] | 444.1 [M + H]+ ESI pos. |
| 138 | (Z)-2-cyano-3-hydroxy-3- (5-methylisoxazol-4-yl)-N- (5-(methylsulfonyl)-4- phenethylpyrimidin-2- yl)acrylamide | 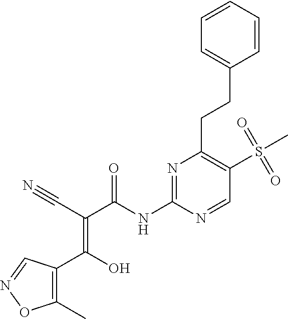 [SMILES: Cc1oncc1/C(O)=C(\C#N)C(=O)Nc1ncc(S(C)(=O)=O)c(CCc2ccccc2)n1] | 454.2 [M + H]+ ESI pos. |
| 139 | (Z)-2-cyano-N-(4-(3- fluorophenyl)-5- (phenylsulfonyl)pyrimidin- 2-yl)-3-hydroxy-3-(5- methylisoxazol-4- yl)acrylamide | 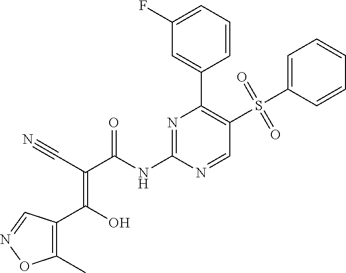 [SMILES: Cc1oncc1/C(O)=C(\C#N)C(=O)Nc1ncc(S(=O)(=O)c2ccccc2)c(-c2cccc(F)c2)n1] | 506.2 [M + H]+ ESI pos. |
| 140 | (Z)-N-(4-(2-chlorophenyl)- 5- (methylsulfonyl)pyrimidin- 2-yl)-2-cyano-3-hydroxy- 3-(5-methylisoxazol-4- yl)acrylamide | 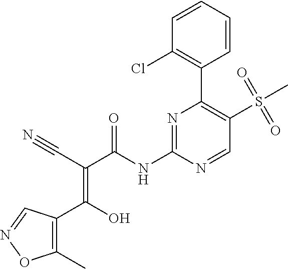 [SMILES: Cc1oncc1/C(O)=C(\C#N)C(=O)Nc1ncc(S(C)(=O)=O)c(-c2ccccc2Cl)n1] | 460.2 [M + H]+ ESI pos. |

In analogy to the procedures described in example 1, examples 141 and 142 were prepared using a suitable aniline in the first step and a suitable isoxazol carbonyl chloride in the second step (table 12).

TABLE 12

| Ex. | Systematic name | Structure | MS |
|---|---|---|---|
| 141 | (Z)-2-cyano-3-(5- ethylisoxazol-4-yl)-3- hydroxy-N-(4- (trifluoromethyl) phenyl)acrylamide | 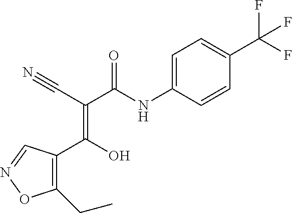 [SMILES: CCc1oncc1/C(O)=C(\C#N)C(=O)Nc1ccc(C(F)(F)F)cc1] | 352.1 [M + H]+ ESI pos. |
| 142 | (Z)-2-cyano-3-hydroxy-3- (isoxazol-4-yl)-N-(4- (trifluoromethyl) phenyl)acrylamide | 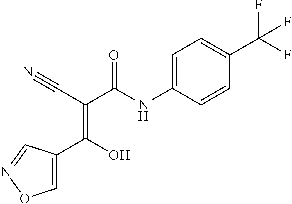 [SMILES: N#C/C(C(=O)Nc1ccc(C(F)(F)F)cc1)=C(/O)c1cnoc1] | 322.2 [M − H]− ESI neg. |

### Example 143

### (Z)-2-Cyano-3-hydroxy-N-[3-(hydroxymethyl)phenyl]-3-(5-methylisoxazol-4-yl)prop-2-enamide

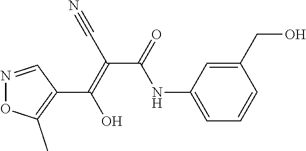 [SMILES: Cc1oncc1/C(O)=C(\C#N)C(=O)Nc1cccc(CO)c1]

### Step 1: 3-[[tert-Butyl(dimethyl)silyl]oxymethyl]aniline

To a solution of 3-aminobenzyl alcohol (500.0 mg, 4.1 mmol) in THE (10 ml) was added tert-butylchlorodimethylsilane (669.27 mg, 4.46 mmol, 1.1 eq), then imidazole (553 mg, 8.1 mmol) at 0° C. The resulting mixture was stirred at 25° C. for 5 hrs. The reaction mixture was concentrated, then diluted with EtOAc and H₂O. The organic phase was washed with brine, dried over Na₂SO₄, filtered and concentrated. The crude product was purified by silica gel chromatography using a petroleum ether/ethyl acetate gradient as eluent to give the title compound (600 mg) as a light-grey oil.

### Steps 2 & 3: (Z)—N-[3-[[tert-butyl(dimethyl)silyl]oxymethyl]phenyl]-2-cyano-3-hydroxy-3-(5-methyl-isoxazol-4-yl)prop-2-enamide

In analogy to the procedures described in example 1, 4-(3-chlorophenyl)-5-(methylsulfonyl)-pyrimidin-2-amine was converted into the title compound in two steps as brown oil.

### Step 4: (Z)-2-Cyano-3-hydroxy-N-[3-(hydroxymethyl)phenyl]-3-(5-methylisoxazol-4-yl)prop-2-enamide

To a solution of (Z)—N-[3-[[tert-butyl(dimethyl)silyl]oxymethyl]phenyl]-2-cyano-3-hydroxy-3-(5-methylisoxazol-4-yl)prop-2-enamide (300 mg, 0.73 mmol) in THF (0.79 ml) was added hydrochloric acid (38.2 mg, 1.05 mmol, 1.4 eq) in water (0.8 ml) at 0° C. The resulting mixture was stirred at 25° C. for 2 hrs. The reaction mixture was concentrated, then diluted with EtOAc and H₂O. The organic phase was washed with brine, dried over Na₂SO₄, filtered and concentrated. The crude product was purified by prep-TLC using dichloromethane/methanol 5% as eluent to provide the title compound (6.2 mg, 2.9% yield) as a colorless oil. MS: 300.1 [M+H]+ ESI pos.

### Example 144

### (3-[[(Z)-2-Cyano-3-hydroxy-3-(5-methylisoxazol-4-yl)prop-2-enoyl]amino]benzoic acid

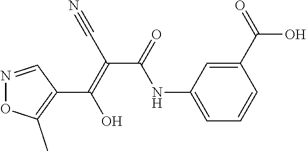 [SMILES: Cc1oncc1/C(O)=C(\C#N)C(=O)Nc1cccc(C(=O)O)c1]

### Steps 1 & 2: tert-Butyl-3-[[(Z)-2-cyano-3-hydroxy-3-(5-methylisoxazol-4-yl)prop-2-enoyl]amino]-benzoate

In analogy to the procedures described in example 1, tert-butyl-3-aminobezoate was converted into the title compound in two steps as yellow solid.

### Step 3: 3-[[(Z)-2-Cyano-3-hydroxy-3-(5-methylisoxazol-4-yl)prop-2-enoyl]amino]benzoic acid

To a solution of tert-butyl-3-[[(Z)-2-cyano-3-hydroxy-3-(5-methylisoxazol-4-yl)prop-2-enoyl]amino]benzoate (150 mg, 0.41 mmol) in DCM (10 ml) was added trifluoroacetic acid (0.3 ml, 4.1 mmol) at 0° C. The resulting mixture was stirred at 0° C. for 3 hrs. The reaction mixture was concentrated in vacuum. The residue was purified by prep-HPLC (column: Phenomenex Synergi C18, 150*25*10 um, mobile phase: A) water (0.1% TFA), B) MeCN (0.1% TFA), 10 min) to provide the title compound (22 mg, 17% yield) off-white solid. MS: 312.2 [M−H]⁻ ESI neg.

### Example 145

### 2-[4-[[(Z)-2-Cyano-3-hydroxy-3-(5-methylisoxazol-4-yl)prop-2-enoyl]amino]phenyl]acetic acid

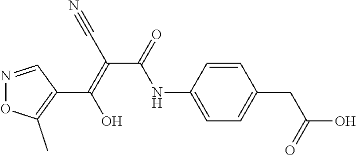 [SMILES: Cc1oncc1/C(O)=C(\C#N)C(=O)Nc1ccc(CC(=O)O)cc1]

### Steps 1 & 2: Methyl 2-[4-[[(Z)-2-cyano-3-hydroxy-3-(5-methylisoxazol-4-yl)prop-2-enoyl]amino]phenyl]acetate

In analogy to the procedures described in example 1, methyl-(4-aminophenyl)acetate was converted into the title compound in two steps as yellow solid.

### Step 3: 2-[4-[[(Z)-2-Cyano-3-hydroxy-3-(5-methylisoxazol-4-yl)prop-2-enoyl]amino]phenyl]acetic acid

To a solution of methyl 2-[4-[[(Z)-2-cyano-3-hydroxy-3-(5-methylisoxazol-4-yl)prop-2-enoyl]amino]phenyl]acetate (50 mg, 0.15 mmol) in methanol (1.25 ml) was added a solution of sodium hydroxide (12 mg, 0.3 mmol, 2.05 eq) in water (0.2 ml), the mixture was stirred at 30° C. for 14 hrs. The reaction mixture was neutralized with 0.5 ml 1N aq. HCl. The crude product was purified by prep-HPLC, then lyophilized to afford the title compound (11.8 mg, 25% yield) as yellow solid. MS: 326.3 [M−H]⁻ ESI neg.

### Example 146

### cGAS Activity Assay—Malachite Green

Compounds were tested for cGAS inhibition in a coupled enzymatic assay based on Phosphate detection by Malachite Green. Final assay conditions were 20 mM TRIS pH 7.5 (Applichem), 5 mM MgCl₂ (Sigma) and 0.01% BSA (Sigma) supplemented with 80 μM ATP (Sigma), 80 μM GTP (Sigma) and 100 nM Interferon Stimulating DNA (ISD) (Microsynth). Recombinantly expressed purified human cGAS (residues 161-522) was used at 25 nM.

All compounds were prepared as 10 mM stock solutions in DMSO and a 16 pt dilution series in DMSO with a dilution factor of 2.5 was prepared. 1 μL of DMSO dilution series was transferred to 32.3 μL reaction buffer, mixed by pipetting up/down, spun for 1 minute at 3000 rpm and was visually inspected for precipitation. 5 μL of 3-fold enzyme stock solution were transferred to an empty 384-well Black/Clear Flat Bottom Polystyrene NBS (Corning) rows 3-24. Rows 1-2 were filled with assay buffer. Plates were spun 10 seconds at 1000 rpm (164×g). 5 μL of compound intermediate dilution was added and mixed by pipetting up/down to rows 3-24. Rows 1-2 were filled with 3.1% DMSO assay buffer. Plates were spun 10 seconds at 1000 rpm (164×g). 5 μL 3-fold Nucleotide/DNA mix was added to all wells to start the reaction. Plates were spun 10 seconds at 1000 rpm (164×g) and incubated for 4 hour at room temperature (RT) in the dark. 5 μL 4 U/ml PPase (Sigma) were added to all wells. Plates spun 10 seconds at 1000 rpm (164×g). 10 μL BioMol green Solution (Enzo Life Sciences) was added to all wells. Plates spun 10 seconds at 1000 rpm (164×g) and incubated 30 minutes at RT in the dark. Absorbance data was collected 620 nm on an EnVision Multilable Reader (Perkin Elmer) and the following measurement settings were used: excitation filter photometric was 620 nm; excitation from the top; measurement height was 1 mm; number of flashes was 30; number of flashes integrated was 1.

All plates are checked for abnormalities and outliers in the Blank Control (no protein, row 1) and the Neutral Control (no compound, row 2) are excluded using the 3*SD rule. Data was normalized to 0 and 100% by Blank and Neutral Control and each curve was fitted and judged using the 4 parameter logistic equation to determine the IC50 for cGAS inhibition.

The results of this assay are provided in Table 13. Table 13 provides IC50 values (μM) for cGAS inhibition obtained for particular examples of the present invention as measured by the above-described assay.

TABLE 13

| Example | IC50 cGAS (μM) |
|---|---|
| 1 | 0.17 |
| 2 | 0.51 |
| 3 | 1.07 |
| 4 | 0.95 |
| 5 | 1.50 |
| 6 | 6.98 |
| 7 | 0.79 |
| 8 | 0.34 |
| 9 | 7.24 |
| 10 | 1.48 |
| 11 | 1.29 |
| 12 | 2.60 |
| 13 | 3.47 |
| 14 | 0.67 |
| 15 | 3.56 |
| 16 | 0.27 |
| 17 | 0.64 |
| 18 | 3.71 |
| 19 | 7.42 |
| 20 | 1.12 |
| 21 | 1.44 |
| 22 | 0.69 |
| 23 | 1.81 |
| 24 | 4.37 |
| 25 | 1.52 |
| 26 | 1.51 |
| 27 | 0.15 |
| 28 | 0.75 |
| 29 | 0.07 |
| 30 | 0.16 |
| 31 | 1.30 |
| 32 | 0.53 |
| 33 | 0.31 |
| 34 | 0.19 |
| 35 | 0.71 |
| 36 | 0.37 |
| 37 | 0.42 |
| 38 | 1.16 |
| 39 | 0.49 |
| 40 | 0.14 |
| 41 | 1.29 |
| 42 | 0.27 |
| 43 | 0.21 |
| 44 | 0.24 |
| 45 | 0.09 |
| 46 | 0.07 |
| 47 | 0.20 |
| 48 | 0.10 |
| 49 | 2.50 |
| 50 | 1.05 |
| 51 | 1.99 |
| 52 | 1.74 |
| 53 | 0.30 |
| 54 | 0.22 |
| 55 | 2.02 |
| 56 | 0.74 |
| 57 | 0.29 |
| 58 | 0.33 |
| 59 | 0.45 |
| 60 | 0.52 |
| 61 | 0.11 |
| 62 | 1.56 |
| 63 | 3.46 |
| 64 | 1.73 |
| 65 | 1.67 |
| 66 | 0.49 |
| 67 | 0.42 |
| 68 | 0.21 |
| 69 | 2.00 |
| 70 | 1.00 |
| 71 | 1.34 |
| 72 | 0.68 |
| 73 | 0.14 |
| 74 | 1.50 |
| 75 | 1.90 |
| 76 | 4.46 |
| 77 | 2.52 |
| 78 | 1.64 |
| 79 | 0.19 |
| 80 | 0.61 |
| 81 | 0.67 |
| 82 | 0.30 |
| 83 | 1.02 |
| 84 | 0.28 |
| 85 | 0.16 |
| 86 | 0.48 |
| 87 | 0.24 |
| 88 | 0.16 |
| 89 | 0.08 |
| 90 | 0.13 |
| 91 | 0.10 |
| 92 | 0.25 |
| 93 | 0.14 |
| 94 | 0.09 |
| 95 | 0.10 |
| 96 | 0.07 |
| 97 | 0.21 |
| 98 | 0.08 |
| 99 | 0.09 |
| 100 | 0.07 |
| 101 | 0.12 |
| 102 | 0.15 |
| 103 | 0.04 |
| 104 | 3.11 |
| 105 | 0.15 |
| 106 | 0.05 |
| 107 | 0.06 |
| 108 | 5.01 |
| 109 | 0.72 |
| 110 | 0.06 |
| 111 | 0.02 |
| 112 | 0.02 |
| 113 | 0.33 |
| 114 | 0.12 |
| 115 | 0.18 |
| 116 | 0.38 |
| 117 | 0.05 |
| 118 | 0.04 |
| 119 | 0.17 |
| 120 | 0.67 |
| 121 | 0.38 |
| 122 | 0.13 |
| 123 | 0.21 |
| 124 | 0.17 |
| 125 | 2.91 |
| 126 | 0.42 |
| 127 | 2.34 |
| 128 | 0.37 |
| 129 | 0.52 |
| 130 | 1.22 |
| 131 | 2.18 |
| 132 | 0.19 |
| 133 | 1.33 |
| 134 | 1.71 |
| 135 | 0.57 |
| 136 | 0.07 |
| 137 | 0.06 |
| 138 | 0.20 |
| 139 | 0.06 |
| 140 | 0.24 |
| 141 | 6.45 |
| 142 | 5.19 |
| 143 | 0.96 |
| 144 | 0.33 |
| 145 | 0.47 |

### Example A

Film coated tablets containing the following ingredients can be manufactured in a conventional manner:

| | |
|---|---|
| Ingredients | Per tablet |

| | | | | |
|---|---|---|---|---|
| Kernel: | | | | |
| Compound of formula (I) | 10.0 | mg | 200.0 | mg |
| Microcrystalline cellulose | 23.5 | mg | 43.5 | mg |
| Lactose hydrous | 60.0 | mg | 70.0 | mg |
| Povidone K30 | 12.5 | mg | 15.0 | mg |
| Sodium starch glycolate | 12.5 | mg | 17.0 | mg |
| Magnesium stearate | 1.5 | mg | 4.5 | mg |
| (Kernel Weight) | 120.0 | mg | 350.0 | mg |
| Film Coat: | | | | |
| Hydroxypropyl methyl cellulose | 3.5 | mg | 7.0 | mg |
| Polyethylene glycol 6000 | 0.8 | mg | 1.6 | mg |
| Talc | 1.3 | mg | 2.6 | mg |
| Iron oxide (yellow) | 0.8 | mg | 1.6 | mg |
| Titan dioxide | 0.8 | mg | 1.6 | mg |

The active ingredient is sieved and mixed with microcrystalline cellulose and the mixture is granulated with a solution of polyvinylpyrrolidone in water. The granulate is then mixed with sodium starch glycolate and magnesium stearate and compressed to yield kernels of 120 or 350 mg respectively. The kernels are lacquered with an aq. solution/suspension of the above mentioned film coat.

### Example B

Capsules containing the following ingredients can be manufactured in a conventional manner:

| Ingredients | Per capsule |
|---|---|
| Compound of formula (I) | 25.0 mg |
| Lactose | 150.0 mg |
| Maize starch | 20.0 mg |
| Talc | 5.0 mg |

The components are sieved and mixed and filled into capsules of size 2.

### Example C

Injection solutions can have the following composition:

| | |
|---|---|
| Compound of formula (I) | 3.0 mg |
| Polyethylene glycol 400 | 150.0 mg |
| Acetic acid | q.s. ad pH 5.0 |
| Water for injection solutions | ad 1.0 ml |

The active ingredient is dissolved in a mixture of Polyethylene glycol 400 and water for injection (part). The pH is adjusted to 5.0 by addition of acetic acid. The volume is adjusted to 1.0 ml by addition of the residual amount of water. The solution is filtered, filled into vials using an appropriate overage and sterilized.

1. A compound of formula (I) 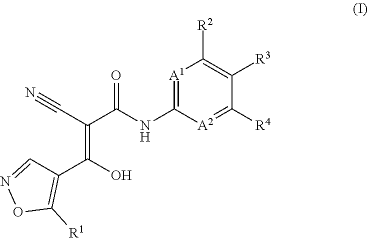
wherein
A¹ and A² are independently selected from nitrogen and —CR⁵—;
R¹ is hydrogen or alkyl;
R² and R⁴ are independently selected from hydrogen, halogen, alkyl, haloalkyl, haloalkyloxy, hydroxyalkyl, alkoxy, phenyl, halophenyl, phenylalkyl, phenylsulfonyl, phenyloxy, cyano, carboxyl, alkoxycarbonyl, alkylsulfonyl, halophenylsulfonylamino, phenylaminocarbonyl and phenylcarbonylamino;
R³ is hydrogen, halogen, haloalkyl, alkoxy, haloalkyloxy, alkoxyalkoxy, cyano, phenyl, phenylalkyl, phenyloxy, alkoxyphenyl, alkylsulfonyl, phenylsulfonyl, phenyl(alkylamino)carbonyl, phenylalkyl(alkylamino)carbonyl, alkoxypyridinylalkyl, phenylsulfanyl, phenylalkylaminocarbonyl, phenylaminocarbonyl, alkoxyalkylsulfonyl, cycloalkyl, cycloalkyloxy, alkoxyalkylaminosulfonyl, alkoxyalkyl(alkylamino)sulfonyl, dialkylaminoalkyl(alkylamino)sulfonyl, alkylaminosulfonyl, phenylsulfinyl, halophenylalkylsulfonyl, cycloalkylalkyl, phenylalkynyl, cycloalkylalkoxy, cycloalkylalkyl(alkylamino)carbonyl, phenylalkylpyrrolidinylaminocarbonyl, halophenyloxy, alkylsulfinyl, (alkylisoxazolylcarbonyl)(dialkylisoxazolyl)aminosulfonyl, dialkylisoxazolylaminosulfonyl, (alkylisoxazolylcarbonyl)(cyanophenyl)aminosulfonyl, cyanophenylaminosulfonyl, phenylsulfonimidoyl, phenylaminosulfonyl, dialkylaminocarbonylalkylaminosulfonyl, morpholinylsulfonyl, pyridinylsulfonyl, cyanophenylsulfonyl, pyrimidinylaminosulfonyl, aminocarbonyl, alkylthiadiazolylaminosulfonyl, alkylcarbonylamino, oxetanyloxy, thiazolylaminosulfonyl, alkylsulfonimidoyl, alkoxyphenylsulfonyl, phenylcarbonyl, alkylaminocarbonyl, dialkylaminocarbonyl, carboxylalkyl, alkoxycarbonyl, alkoxycarbonylalkyl, pyrrolidinylsulfonyl, dialkylaminosulfonyl, 1H-pyrrolyl, haloalkylphenyloxy, halophenylsulfonyl, alkylsulfanyl, halophenylsulfanyl or alkylcarbonyl; and
R⁵ is hydrogen, halogen, alkyl or haloalkyl;
or a pharmaceutically acceptable salt, stereoisomer or tautomer thereof.

2. A compound according to claim 1, wherein A¹ and A² are both nitrogen or —CR⁵— at the same time.

3. A compound according to claim 1 or 2, wherein R¹ is alkyl.

4. A compound according to any one of claims 1 to 3, wherein R¹ is methyl.

5. A compound according to any one of claims 1 to 4, wherein R² and R⁴ are independently selected from hydrogen and halophenyl.

6. A compound according to any one of claims 1 to 5, wherein R² and R⁴ are independently selected from hydrogen, chlorophenyl and fluorophenyl.

7. A compound according to any one of claims 1 to 6, wherein R³ is phenylsulfonyl, alkylsulfonyl, halogen, phenylsulfinyl, phenylalkylaminocarbonyl, alkoxyalkylsulfonyl, alkoxyalkylaminosulfonyl, alkoxyalkyl(alkylamino)sulfonyl, phenylalkyl(alkylamino)carbonyl, phenyl(alkylamino)carbonyl, alkoxy, dialkylaminosulfonyl, haloalkyl, alkoxyalkoxy or pyrimidinylaminosulfonyl.

8. A compound according to any one of claims 1 to 7, wherein R³ is phenylsulfonyl, methylsulfonyl, ethylsulfonyl, bromo, phenylsulfinyl, phenylmethylaminocarbonyl, methoxyethylsulfonyl, methoxyethylaminosulfonyl, methoxyethyl(methylamino)sulfonyl, phenylmethyl(methylamino)carbonyl, phenyl(methylamino)carbonyl, methoxy, ethoxy, dimethylaminosulfonyl, trifluoromethyl, methoxyethoxy or pyrimidinylaminosulfonyl.

9. A compound according to any one of claims 1 to 8 selected from
(Z)—N-(4-(2-chlorophenyl)-5-(methylsulfonyl)pyrimidin-2-yl)-2-cyano-3-hydroxy-3-(5-methylisoxazol-4-yl)acrylamide;
(Z)-2-cyano-N-(4-(3-fluorophenyl)-5-(phenylsulfonyl)pyrimidin-2-yl)-3-hydroxy-3-(5-methylisoxazol-4-yl)acrylamide;
(Z)—N-(4-(3-chlorophenyl)-5-(methylsulfonyl)pyrimidin-2-yl)-2-cyano-3-hydroxy-3-(5-methylisoxazol-4-yl)acrylamide;
(Z)—N-(4-chloro-6-((4-chlorophenyl)sulfonamido)-5-phenylpyrimidin-2-yl)-2-cyano-3-hydroxy-3-(5-methylisoxazol-4-yl)acrylamide;
(Z)-2-(2-cyano-3-hydroxy-3-(5-methylisoxazol-4-yl)acrylamido)-N-methyl-N,4-diphenylpyrimidine-5-carboxamide;
(Z)-2-(2-cyano-3-hydroxy-3-(5-methylisoxazol-4-yl)acrylamido)-N-methyl-4-phenethyl-N-phenylpyrimidine-5-carboxamide;
(Z)—N-benzyl-2-(2-cyano-3-hydroxy-3-(5-methylisoxazol-4-yl)acrylamido)-N-methyl-4-phenethylpyrimidine-5-carboxamide;
(Z)—N-benzyl-2-(2-cyano-3-hydroxy-3-(5-methylisoxazol-4-yl)acrylamido)-N-methyl-4-phenylpyrimidine-5-carboxamide;
(Z)—N-(5-bromopyrimidin-2-yl)-2-cyano-3-hydroxy-3-(5-methylisoxazol-4-yl)acrylamide;
(Z)-2-cyano-3-hydroxy-3-(5-methylisoxazol-4-yl)-N-(5-(methylsulfonyl)-4-phenethylpyrimidin-2-yl)acrylamide;
(Z)-2-cyano-3-hydroxy-N-(5-methoxy-4-phenylpyrimidin-2-yl)-3-(5-methylisoxazol-4-yl)acrylamide;
(Z)-2-cyano-3-hydroxy-3-(5-methylisoxazol-4-yl)-N-(5-phenethyl-4-(trifluoromethyl)pyrimidin-2-yl)acrylamide;
(Z)-2-cyano-3-hydroxy-N-(5-(2-(6-methoxypyridin-3-yl)ethyl)-4-(trifluoromethyl)pyrimidin-2-yl)-3-(5-methylisoxazol-4-yl)acrylamide;
(Z)-2-cyano-3-hydroxy-N-(5-methoxy-4-phenethylpyrimidin-2-yl)-3-(5-methylisoxazol-4-yl)acrylamide;
(Z)-2-cyano-3-hydroxy-N-(5-(2-methoxyethoxy)-4-phenethylpyrimidin-2-yl)-3-(5-methylisoxazol-4-yl)acrylamide;
(Z)-2-cyano-3-hydroxy-N-(5-(2-methoxyethoxy)-4-phenylpyrimidin-2-yl)-3-(5-methylisoxazol-4-yl)acrylamide;
(Z)—N-[5-(benzenesulfinyl)pyrimidin-2-yl]-2-cyano-3-hydroxy-3-(5-methylisoxazol-4-yl)prop-2-enamide;
(Z)—N-[5-(benzenesulfonyl)pyrimidin-2-yl]-2-cyano-3-hydroxy-3-(5-methylisoxazol-4-yl)prop-2-enamide;
N-benzyl-2-[[(Z)-2-cyano-3-hydroxy-3-(5-methylisoxazol-4-yl)prop-2-enoyl]amino]pyrimidine-5-carboxamide;
2-[[(Z)-2-cyano-3-hydroxy-3-(5-methylisoxazol-4-yl)prop-2-enoyl]amino]-N-phenyl-pyrimidine-5-carboxamide;
(Z)-2-cyano-3-hydroxy-3-(5-methylisoxazol-4-yl)-N-(4-(2-phenylpropan-2-yl)phenyl)acrylamide;
(Z)-2-cyano-N-(5-(ethylsulfonyl)pyrimidin-2-yl)-3-hydroxy-3-(5-methylisoxazol-4-yl)acrylamide;
(Z)-2-cyano-3-hydroxy-3-(5-methylisoxazol-4-yl)-N-(5-phenethylpyrimidin-2-yl)acrylamide;
(Z)-2-cyano-3-hydroxy-N-(5-((2-methoxyethyl)sulfonyl)pyrimidin-2-yl)-3-(5-methylisoxazol-4-yl)acrylamide;
(Z)—N-(4-(4-chlorophenyl)-5-(cyclopropylmethoxy)pyrimidin-2-yl)-2-cyano-3-hydroxy-3-(5-methylisoxazol-4-yl)acrylamide;
(Z)—N-(4-(4-chlorophenyl)-5-(2,2,2-trifluoroethoxy)pyrimidin-2-yl)-2-cyano-3-hydroxy-3-(5-methylisoxazol-4-yl)acrylamide;
(Z)-2-cyano-3-hydroxy-N-(5-(N-(2-methoxyethyl)sulfamoyl)pyrimidin-2-yl)-3-(5-methylisoxazol-4-yl)acrylamide;
(Z)-2-cyano-3-hydroxy-N-(5-(N-(2-methoxyethyl)-N-methylsulfamoyl)pyrimidin-2-yl)-3-(5-methylisoxazol-4-yl)acrylamide;
(Z)-2-cyano-N-(4-(N-(2-(dimethylamino)ethyl)-N-methylsulfamoyl)phenyl)-3-hydroxy-3-(5-methylisoxazol-4-yl)acrylamide;
(Z)-2-cyano-N-(4-(3-fluorophenyl)-5-(methylsulfonyl)pyrimidin-2-yl)-3-hydroxy-3-(5-methylisoxazol-4-yl)acrylamide;
(Z)-2-cyano-N-(4-(2-fluoroethoxy)pyrimidin-2-yl)-3-hydroxy-3-(5-methylisoxazol-4-yl)acrylamide;
N-benzyl-2-[[(Z)-2-cyano-3-hydroxy-3-(5-methylisoxazol-4-yl)prop-2-enoyl]amino]-N-methyl-pyrimidine-5-carboxamide;
2-[[(Z)-2-cyano-3-hydroxy-3-(5-methylisoxazol-4-yl)prop-2-enoyl]amino]-N-methyl-N-phenyl-pyrimidine-5-carboxamide;
(Z)-2-cyano-N-(5-ethoxypyrimidin-2-yl)-3-hydroxy-3-(5-methylisoxazol-4-yl)acrylamide;
(Z)-2-cyano-3-hydroxy-3-(5-methylisoxazol-4-yl)-N-(5-phenylsulfanylpyrimidin-2-yl)prop-2-enamide;
(Z)-2-cyano-N-(5-(N,N-dimethylsulfamoyl)pyrimidin-2-yl)-3-hydroxy-3-(5-methylisoxazol-4-yl)acrylamide;
(Z)-2-cyano-3-hydroxy-3-(5-methylisoxazol-4-yl)-N-(5-(phenylsulfonyl)pyridin-2-yl)acrylamide;
(Z)-2-cyano-3-hydroxy-3-(5-methylisoxazol-4-yl)-N-(5-(trifluoromethyl)pyrimidin-2-yl)acrylamide;
(Z)—N-(5-(N-(tert-butyl)sulfamoyl)pyridin-2-yl)-2-cyano-3-hydroxy-3-(5-methylisoxazol-4-yl)acrylamide;
(Z)-2-cyano-N-[3-fluoro-5-(trifluoromethyl)-2-pyridyl]-3-hydroxy-3-(5-methylisoxazol-4-yl)prop-2-enamide;
(Z)-2-cyano-3-hydroxy-3-(5-methylisoxazol-4-yl)-N-(5-(methylsulfonyl)pyridin-2-yl)acrylamide;
(Z)-2-cyano-3-hydroxy-3-(5-methylisoxazol-4-yl)-N-(5-(phenylsulfinyl)pyridin-2-yl)acrylamide;
(Z)-2-cyano-3-hydroxy-N-(5-(2-methoxyethoxy)pyrimidin-2-yl)-3-(5-methylisoxazol-4-yl)acrylamide;
(Z)-2-cyano-3-hydroxy-N-(4-(N-(2-methoxyethyl)-N-methylsulfamoyl)phenyl)-3-(5-methylisoxazol-4-yl)acrylamide;
(Z)—N-(5-((2-chlorobenzyl)sulfonyl)pyridin-2-yl)-2-cyano-3-hydroxy-3-(5-methylisoxazol-4-yl)acrylamide;
(Z)-2-cyano-N-(5-(cyclohexylmethyl)pyrimidin-2-yl)-3-hydroxy-3-(5-methylisoxazol-4-yl)acrylamide;
(Z)-2-cyano-3-hydroxy-3-(5-methylisoxazol-4-yl)-N-(4-phenethylpyrimidin-2-yl)acrylamide;
(Z)-2-cyano-3-hydroxy-3-(5-methylisoxazol-4-yl)-N-(5-(phenylethynyl)pyrimidin-2-yl)acrylamide;
(Z)-2-cyano-N-(5-(cyclopentylmethoxy)pyrimidin-2-yl)-3-hydroxy-3-(5-methylisoxazol-4-yl)acrylamide;
(Z)—N-(3-chloro-5-(trifluoromethyl)pyridin-2-yl)-2-cyano-3-hydroxy-3-(5-methylisoxazol-4-yl)acrylamide;
(Z)-2-cyano-3-hydroxy-3-(5-methylisoxazol-4-yl)-N-(5-phenoxypyridin-2-yl)acrylamide;
(Z)-2-cyano-3-hydroxy-3-(5-methylisoxazol-4-yl)-N-(5-phenoxypyrimidin-2-yl)acrylamide;
(Z)-2-cyano-3-hydroxy-3-(5-methylisoxazol-4-yl)-N-(5-phenylpyrimidin-2-yl)acrylamide;
(Z)-2-cyano-3-hydroxy-N-(5-(4-methoxyphenyl)pyrimidin-2-yl)-3-(5-methylisoxazol-4-yl)acrylamide;
(Z)-2-cyano-N-(5-cyclohexylpyrimidin-2-yl)-3-hydroxy-3-(5-methylisoxazol-4-yl)acrylamide;
(Z)-2-cyano-N-(3-cyano-4-(trifluoromethyl)phenyl)-3-hydroxy-3-(5-methylisoxazol-4-yl)acrylamide;
(Z)-2-chloro-4-(2-cyano-3-hydroxy-3-(5-methylisoxazol-4-yl)acrylamido)-N-(cyclohexylmethyl)-N-methylbenzamide;
(Z)-2-cyano-3-hydroxy-N-(3-methoxy-4-(trifluoromethyl)phenyl)-3-(5-methylisoxazol-4-yl)acrylamide;
(Z)-2-cyano-3-hydroxy-N-(3-methyl-4-(trifluoromethyl)phenyl)-3-(5-methylisoxazol-4-yl)acrylamide;
(Z)—N-(4-benzylphenyl)-2-cyano-3-hydroxy-3-(5-methylisoxazol-4-yl)acrylamide;
(Z)-2-cyano-N-(5-(cyclohexyloxy)pyrimidin-2-yl)-3-hydroxy-3-(5-methylisoxazol-4-yl)acrylamide;
(Z)—N-benzyl-4-(2-cyano-3-hydroxy-3-(5-methylisoxazol-4-yl)acrylamido)benzamide;
(Z)—N-(3-(2-cyano-3-hydroxy-3-(5-methylisoxazol-4-yl)acrylamido)phenyl)benzamide;
(S,Z)—N-(1-benzylpyrrolidin-3-yl)-4-(2-cyano-3-hydroxy-3-(5-methylisoxazol-4-yl)acrylamido)benzamide hydrochloride;
(Z)-3-(2-cyano-3-hydroxy-3-(5-methylisoxazol-4-yl)acrylamido)-N-phenylbenzamide;
(Z)-2-cyano-3-hydroxy-3-(5-methylisoxazol-4-yl)-N-(4-phenylpyrimidin-2-yl)acrylamide;
(Z)-4-(2-cyano-3-hydroxy-3-(5-methylisoxazol-4-yl)acrylamido)-N-phenylbenzamide;
(Z)-2-cyano-3-hydroxy-3-(5-methylisoxazol-4-yl)-N-(5-(trifluoromethyl)pyridin-2-yl)acrylamide;
(Z)—N-(3-chloro-4-(trifluoromethyl)phenyl)-2-cyano-3-hydroxy-3-(5-methylisoxazol-4-yl)acrylamide;
(Z)-2-cyano-3-hydroxy-3-(5-methylisoxazol-4-yl)-N-(4-phenylphenyl)prop-2-enamide;
(Z)—N-(4-(4-chlorophenoxy)phenyl)-2-cyano-3-hydroxy-3-(5-methylisoxazol-4-yl)acrylamide;
(Z)-2-cyano-3-hydroxy-3-(5-methylisoxazol-4-yl)-N-(4-phenoxyphenyl)acrylamide;
(Z)-2-cyano-3-hydroxy-3-(5-methylisoxazol-4-yl)-N-(4-methylsulfinylphenyl)prop-2-enamide;
(Z)-2-cyano-3-hydroxy-3-(5-methylisoxazol-4-yl)-N-(4-methylsulfonylphenyl)prop-2-enamide;
(Z)—N-(2-chloro-4-methylsulfonyl-phenyl)-2-cyano-3-hydroxy-3-(5-methylisoxazol-4-yl)prop-2-enamide;
(Z)—N-((4-(2-cyano-3-hydroxy-3-(5-methylisoxazol-4-yl)acrylamido)phenyl)sulfonyl)-N-(3,5-dimethylisoxazol-4-yl)-5-methylisoxazole-4-carboxamide;
(Z)-2-cyano-N-(4-(N-(3,5-dimethylisoxazol-4-yl)sulfamoyl)phenyl)-3-hydroxy-3-(5-methylisoxazol-4-yl)acrylamide;
(Z)—N-((4-(2-cyano-3-hydroxy-3-(5-methylisoxazol-4-yl)acrylamido)phenyl)sulfonyl)-N-(4-cyanophenyl)-5-methylisoxazole-4-carboxamide;
(Z)-2-cyano-N-(4-(N-(4-cyanophenyl)sulfamoyl)phenyl)-3-hydroxy-3-(5-methylisoxazol-4-yl)acrylamide;
(Z)-2-cyano-3-hydroxy-3-(5-methylisoxazol-4-yl)-N-(4-(phenylsulfonimidoyl)phenyl)acrylamide;
(Z)—N-(2-chloro-4-(trifluoromethyl)phenyl)-2-cyano-3-hydroxy-3-(5-methylisoxazol-4-yl)acrylamide;
(Z)-2-cyano-3-hydroxy-3-(5-methylisoxazol-4-yl)-N-(4-(phenylthio)phenyl)acrylamide;
(Z)-2-cyano-3-hydroxy-3-(5-methylisoxazol-4-yl)-N-(4-(N-phenylsulfamoyl)phenyl)acrylamide;
(Z)-2-cyano-N-(4-(N-(2-(diethylamino)-2-oxoethyl)sulfamoyl)phenyl)-3-hydroxy-3-(5-methylisoxazol-4-yl)acrylamide;
(Z)-2-cyano-3-hydroxy-3-(5-methylisoxazol-4-yl)-N-(3-phenoxyphenyl)acrylamide;
(Z)-2-cyano-3-hydroxy-3-(5-methylisoxazol-4-yl)-N-(3-phenylphenyl)prop-2-enamide;
(Z)-2-cyano-3-hydroxy-N-(2-methyl-4-(trifluoromethyl)phenyl)-3-(5-methylisoxazol-4-yl)acrylamide;
(Z)-2-cyano-3-hydroxy-3-(5-methylisoxazol-4-yl)-N-(3-(phenylsulfonyl)phenyl)acrylamide;
(Z)-2-cyano-3-hydroxy-3-(5-methylisoxazol-4-yl)-N-(4-(N-propylsulfamoyl)phenyl)acrylamide;
(Z)-2-cyano-3-hydroxy-3-(5-methylisoxazol-4-yl)-N-(4-(morpholinosulfonyl)phenyl)acrylamide;
(Z)-2-cyano-3-hydroxy-N-(5-methoxypyrimidin-2-yl)-3-(5-methylisoxazol-4-yl)prop-2-enamide;
(Z)-2-cyano-3-hydroxy-3-(5-methylisoxazol-4-yl)-N-(4-(pyridin-3-ylsulfonyl)phenyl)acrylamide;
(Z)-2-cyano-N-(4-((2-cyanophenyl)sulfonyl)phenyl)-3-hydroxy-3-(5-methylisoxazol-4-yl)acrylamide;
(Z)-2-cyano-N-(4-((4-cyanophenyl)sulfonyl)phenyl)-3-hydroxy-3-(5-methylisoxazol-4-yl)acrylamide;
(Z)-2-cyano-3-(5-ethylisoxazol-4-yl)-3-hydroxy-N-(4-(trifluoromethyl)phenyl)acrylamide;
(Z)-2-cyano-3-hydroxy-3-(5-methylisoxazol-4-yl)-N-[4-(pyrimidin-2-ylsulfamoyl)phenyl]prop-2-enamide;
4-[[(Z)-2-cyano-3-hydroxy-3-(5-methylisoxazol-4-yl)prop-2-enoyl]amino]benzamide;
(Z)-2-cyano-3-hydroxy-3-(5-methylisoxazol-4-yl)-N-[4-[(5-methyl-1,3,4-thiadiazol-2-yl)sulfamoyl]phenyl]prop-2-enamide;
(Z)—N-(4-acetamidophenyl)-2-cyano-3-hydroxy-3-(5-methylisoxazol-4-yl)prop-2-enamide;
(Z)-2-cyano-3-hydroxy-3-(5-methylisoxazol-4-yl)-N-[4-(oxetan-3-yloxy)phenyl]prop-2-enamide;
3-[[(Z)-2-cyano-3-hydroxy-3-(5-methylisoxazol-4-yl)prop-2-enoyl]amino]benzoic acid;
(Z)-2-cyano-3-hydroxy-3-(5-methylisoxazol-4-yl)-N-[4-(thiazol-2-ylsulfamoyl)phenyl]prop-2-enamide;
(Z)-2-cyano-3-hydroxy-3-(5-methylisoxazol-4-yl)-N-(4-(S-methylsulfonimidoyl)phenyl)acrylamide;
(Z)-2-cyano-3-hydroxy-N-(4-((4-methoxyphenyl)sulfonyl)phenyl)-3-(5-methylisoxazol-4-yl)acrylamide;
(Z)—N-(4-benzoylphenyl)-2-cyano-3-hydroxy-3-(5-methylisoxazol-4-yl)prop-2-enamide;
(Z)-2-cyano-3-hydroxy-N-(5-methoxy-2-pyridyl)-3-(5-methylisoxazol-4-yl)prop-2-enamide;
4-[[(Z)-2-cyano-3-hydroxy-3-(5-methylisoxazol-4-yl)prop-2-enoyl]amino]-N-methylbenzamide;
2-[4-[[(Z)-2-cyano-3-hydroxy-3-(5-methylisoxazol-4-yl)prop-2-enoyl]amino]phenyl]acetic acid;
4-[[(Z)-2-cyano-3-hydroxy-3-(5-methylisoxazol-4-yl)prop-2-enoyl]amino]-N,N-dimethylbenzamide;
(Z)-2-cyano-3-hydroxy-3-(5-methylisoxazol-4-yl)-N-(4-(phenylsulfinyl)phenyl)acrylamide;
(Z)-2-cyano-N-(3-cyanophenyl)-3-hydroxy-3-(5-methylisoxazol-4-yl)prop-2-enamide;
methyl 2-[4-[[(Z)-2-cyano-3-hydroxy-3-(5-methylisoxazol-4-yl)prop-2-enoyl]amino]phenyl]acetate;
(Z)-2-cyano-3-hydroxy-N-(4-methoxy-3-methyl-phenyl)-3-(5-methylisoxazol-4-yl)prop-2-enamide;
(Z)-2-cyano-3-hydroxy-3-(5-methylisoxazol-4-yl)-N-(4-(phenylsulfonyl)phenyl)acrylamide;
(Z)-2-cyano-3-hydroxy-N-(4-(N-(2-methoxyethyl)sulfamoyl)phenyl)-3-(5-methylisoxazol-4-yl)acrylamide;
(Z)-2-cyano-N-(4-ethoxyphenyl)-3-hydroxy-3-(5-methylisoxazol-4-yl)prop-2-enamide;
(Z)-2-cyano-3-hydroxy-3-(5-methylisoxazol-4-yl)-N-(4-(pyrrolidin-1-ylsulfonyl)phenyl)acrylamide;
(Z)-2-cyano-3-hydroxy-3-(5-methylisoxazol-4-yl)-N-(3-(methylsulfonyl)phenyl)acrylamide;
(Z)-2-cyano-N-(4-(N,N-dimethylsulfamoyl)phenyl)-3-hydroxy-3-(5-methylisoxazol-4-yl)acrylamide;
(Z)-2-cyano-3-hydroxy-N-[3-(hydroxymethyl)phenyl]-3-(5-methylisoxazol-4-yl)prop-2-enamide;
methyl 3-[[(Z)-2-cyano-3-hydroxy-3-(5-methylisoxazol-4-yl)prop-2-enoyl]amino]benzoate;
(Z)-2-cyano-3-hydroxy-3-(isoxazol-4-yl)-N-(4-(trifluoromethyl)phenyl)acrylamide;
(Z)—N-(3-chloro-4-(4-chlorophenoxy)phenyl)-2-cyano-3-hydroxy-3-(5-methylisoxazol-4-yl)acrylamide;
(Z)—N-(4-bromo-3-methylphenyl)-2-cyano-3-hydroxy-3-(5-methylisoxazol-4-yl)acrylamide;
(Z)-2-cyano-N-(4-cyano-2-methylphenyl)-3-hydroxy-3-(5-methylisoxazol-4-yl)acrylamide;
(Z)—N-(4-(1H-pyrrol-1-yl)phenyl)-2-cyano-3-hydroxy-3-(5-methylisoxazol-4-yl)acrylamide;
(Z)—N-(4-chlorophenyl)-2-cyano-3-hydroxy-3-(5-methylisoxazol-4-yl)acrylamide;
(Z)-2-cyano-3-hydroxy-N-(3-methyl-4-(4-(trifluoromethyl)phenoxy)phenyl)-3-(5-methylisoxazol-4-yl)acrylamide;
(Z)-2-cyano-3-hydroxy-3-(5-methylisoxazol-4-yl)-N-(4-(3-(trifluoromethyl)phenoxy)phenyl)acrylamide;
(Z)-2-cyano-3-hydroxy-N-(4-methoxyphenyl)-3-(5-methylisoxazol-4-yl)acrylamide;
(Z)—N-(4-((4-chlorophenyl)sulfonyl)phenyl)-2-cyano-3-hydroxy-3-(5-methylisoxazol-4-yl)acrylamide;
(Z)—N-(4-chloro-3-(trifluoromethyl)phenyl)-2-cyano-3-hydroxy-3-(5-methylisoxazol-4-yl)acrylamide;
(Z)-2-cyano-3-hydroxy-3-(5-methylisoxazol-4-yl)-N-(4-(methylthio)phenyl)acrylamide;
(Z)—N-(4-((4-chlorophenyl)thio)phenyl)-2-cyano-3-hydroxy-3-(5-methylisoxazol-4-yl)acrylamide;
(Z)-2-cyano-N-(3,4-dichlorophenyl)-3-hydroxy-3-(5-methylisoxazol-4-yl)acrylamide;
(Z)-2-cyano-3-hydroxy-3-(5-methylisoxazol-4-yl)-N-(4-(trifluoromethoxy)phenyl)acrylamide;
(Z)-2-cyano-N-(4-fluorophenyl)-3-hydroxy-3-(5-methylisoxazol-4-yl)acrylamide;
(Z)-2-cyano-3-hydroxy-3-(5-methylisoxazol-4-yl)-N-(2-(trifluoromethyl)phenyl)acrylamide;
methyl (Z)-4-(2-cyano-3-hydroxy-3-(5-methylisoxazol-4-yl)acrylamido)benzoate;
(Z)-2-cyano-N-(4-cyanophenyl)-3-hydroxy-3-(5-methylisoxazol-4-yl)acrylamide;
(Z)—N-(3,5-bis(trifluoromethyl)phenyl)-2-cyano-3-hydroxy-3-(5-methylisoxazol-4-yl)acrylamide;
(Z)-2-cyano-3-hydroxy-3-(5-methylisoxazol-4-yl)-N-[3-(trifluoromethyl)phenyl]prop-2-enamide;
(Z)-2-cyano-3-hydroxy-3-(5-methylisoxazol-4-yl)-N-(4-(trifluoromethyl)phenyl)acrylamide;
(Z)-2-cyano-3-hydroxy-3-(5-methylisoxazol-4-yl)-N-phenylacrylamide; and
(Z)—N-(4-acetylphenyl)-2-cyano-3-hydroxy-3-(5-methylisoxazol-4-yl)prop-2-enamide;
or a pharmaceutically acceptable salt, stereoisomer or tautomer thereof.

10. A compound according to any one of claims 1 to 9 selected from
(Z)-2-cyano-N-(4-(3-fluorophenyl)-5-(phenylsulfonyl)pyrimidin-2-yl)-3-hydroxy-3-(5-methylisoxazol-4-yl)acrylamide;
(Z)—N-(4-(3-chlorophenyl)-5-(methylsulfonyl)pyrimidin-2-yl)-2-cyano-3-hydroxy-3-(5-methylisoxazol-4-yl)acrylamide;
(Z)—N-(5-bromopyrimidin-2-yl)-2-cyano-3-hydroxy-3-(5-methylisoxazol-4-yl)acrylamide;
(Z)—N-[5-(benzenesulfinyl)pyrimidin-2-yl]-2-cyano-3-hydroxy-3-(5-methylisoxazol-4-yl)prop-2-enamide;
(Z)—N-[5-(benzenesulfonyl)pyrimidin-2-yl]-2-cyano-3-hydroxy-3-(5-methylisoxazol-4-yl)prop-2-enamide;
N-benzyl-2-[[(Z)-2-cyano-3-hydroxy-3-(5-methylisoxazol-4-yl)prop-2-enoyl]amino]pyrimidine-5-carboxamide;
(Z)-2-cyano-N-(5-(ethylsulfonyl)pyrimidin-2-yl)-3-hydroxy-3-(5-methylisoxazol-4-yl)acrylamide;
(Z)-2-cyano-3-hydroxy-N-(5-((2-methoxyethyl)sulfonyl)pyrimidin-2-yl)-3-(5-methylisoxazol-4-yl)acrylamide;
(Z)-2-cyano-3-hydroxy-N-(5-(N-(2-methoxyethyl)sulfamoyl)pyrimidin-2-yl)-3-(5-methylisoxazol-4-yl)acrylamide;
(Z)-2-cyano-3-hydroxy-N-(5-(N-(2-methoxyethyl)-N-methylsulfamoyl)pyrimidin-2-yl)-3-(5-methylisoxazol-4-yl)acrylamide;
(Z)-2-cyano-N-(4-(3-fluorophenyl)-5-(methylsulfonyl)pyrimidin-2-yl)-3-hydroxy-3-(5-methylisoxazol-4-yl)acrylamide;
N-benzyl-2-[[(Z)-2-cyano-3-hydroxy-3-(5-methylisoxazol-4-yl)prop-2-enoyl]amino]-N-methyl-pyrimidine-5-carboxamide;
2-[[(Z)-2-cyano-3-hydroxy-3-(5-methylisoxazol-4-yl)prop-2-enoyl]amino]-N-methyl-N-phenyl-pyrimidine-5-carboxamide;
(Z)-2-cyano-N-(5-ethoxypyrimidin-2-yl)-3-hydroxy-3-(5-methylisoxazol-4-yl)acrylamide;
(Z)-2-cyano-N-(5-(N,N-dimethylsulfamoyl)pyrimidin-2-yl)-3-hydroxy-3-(5-methylisoxazol-4-yl)acrylamide;
(Z)-2-cyano-3-hydroxy-3-(5-methylisoxazol-4-yl)-N-(5-(trifluoromethyl)pyrimidin-2-yl)acrylamide;
(Z)-2-cyano-3-hydroxy-N-(5-(2-methoxyethoxy)pyrimidin-2-yl)-3-(5-methylisoxazol-4-yl)acrylamide;
(Z)-2-cyano-3-hydroxy-N-(5-methoxypyrimidin-2-yl)-3-(5-methylisoxazol-4-yl)prop-2-enamide;
(Z)-2-cyano-3-hydroxy-3-(5-methylisoxazol-4-yl)-N-[4-(pyrimidin-2-ylsulfamoyl)phenyl]prop-2-enamide; and
(Z)-2-cyano-3-hydroxy-N-(4-(N-(2-methoxyethyl)sulfamoyl)phenyl)-3-(5-methylisoxazol-4-yl)acrylamide;
or a pharmaceutically acceptable salt, stereoisomer or tautomer thereof.

11. A process for the preparation of a compound according to any one of claims 1 to 10, comprising the coupling of a compound of formula (B1) 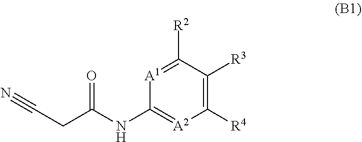
with a compound of formula (B2) 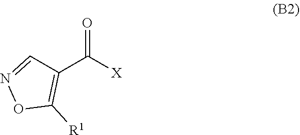
in the presence of a base;
wherein R¹-R⁴, A¹ and A² are as defined in any one of claims 1 to 10 and X is a leaving group, such as halogen, mesylat or tosylat.

12. A compound according to any one of claims 1 to 10, when manufactured according to a process of claim 11.

13. A compound according to any one of claims 1 to 10, for use as therapeutically active substance.

14. A pharmaceutical composition comprising a compound in accordance with any one of claims 1 to 10 and a therapeutically inert carrier.

15. The use of a compound according to any one of claims 1 to 10 for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

16. The use of a compound according to any one of claims 1 to 10 for the preparation of a medicament for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

17. A compound according to any one of claims 1 to 10 for use in the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

18. A method for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS), which method comprises administering an effective amount of a compound as defined in any one of claims 1 to 10 to a patient in need thereof.

19. The invention as hereinbefore described.
